(19) United States
(12) Patent Application Publication
(10) Pub. No.: US 2014/0213779 A1
(43) Pub. Date: Jul. 31, 2014

(54) RIBOSWITCHES

(71) Applicants: Neil Dixon, Manchester (GB); Jason Micklefield, Manchester (GB)

(72) Inventors: Neil Dixon, Manchester (GB); Jason Micklefield, Manchester (GB)

(73) Assignee: THE UNIVERSITY OF MANCHESTER, Manchester (GB)

(21) Appl. No.: 14/116,727

(22) PCT Filed: May 10, 2012
(86) PCT No.: PCT/GB2012/051029

§ 371 Date: Nov. 8, 2013

(30) Foreign Application Priority Data
May 10, 2011 (GB) 1107768.2

(51) Int. Cl. C12N 15/115

(52) U.S. Cl. CPC C12N 15/115
USPC 536/24.1

(57) ABSTRACT

The present invention relates to a system comprising a genetic construct a riboswitch operably linked to a regulatory sequence, and a second genetic construct a coding sequence whose expression is capable of being regulated by a gene product of the first construct. Also provided is a genetic construct comprising one or more riboswitches for regulation of gene expression, wherein preferably a spacer sequence is provided downstream of the riboswitch to enhance expression of a coding sequence which is operably linked to a riboswitch. Ligands, kits, methods, host cells and expression systems are also provided.

The present invention relates to genetic constructs comprising a riboswitch for regulation of gene expression; kits comprising a genetic construct of the invention and optionally one or more ligands of a riboswitch; and methods of regulating expression of a coding sequence, using a genetic construct of the invention and a ligand of the riboswitch. Also provided are host cells or expression systems comprising a genetic construct of the invention and optionally a ligand of the riboswitch of the genetic construct.

### BACKGROUND

A number of small-molecule inducible expression systems have been developed for bacteria. Notably, systems based on the lactose (lac), arabinose (ara) and tetracycline (tet) operons have been extensively utilised for the production of recombinant proteins, including biopharmaceuticals and industrial biocatalysts, and are also widely used for in vivo gene function analysis. For example, by placing a second copy of a gene under the control of an inducible promoter, then deleting the original gene from the chromosome, it is possible to create conditional mutants which can be used to explore the function of essential gene products in bacteria. Similarly, inducible expression systems have been used to control antisense RNA-mediated down-regulation of critical genes in pathogenic bacteria, allowing for the development of sensitive screens for new anti-microbial agents. Inducible expression systems have become indispensable tools in synthetic biology and have been used to control genetic circuits that can function as sensors, switches and oscillators. The expression of novel metabolic pathways, under the control of inducible promoters, has been employed for the production of fine chemicals as well as natural products including drug precursors and fatty acids for biofuels. Expression systems triggered by specific organic pollutants are also being developed for bioremediation applications.

Despite many notable successes, the commonly used regulatory systems do have limitations. Firstly, some of these systems exhibit all-or-none expression, where a subpopulation of cells are fully induced, whilst others remain un-induced. This can be problematic for many applications, including gene function analysis where tuneable gene expression, through titration of the inducer, is preferred. A number of the promoter based systems are also “leaky”, exhibiting high basal levels of expression, which may be undesirable particularly for the expression of toxic genes. Some systems, such as lac and ara, exhibit cross-talk which limits their use in synthetic biology applications, where it is desirable to control the simultaneous and differential expression of multiple genes using distinct inducers. Some systems are limited by undesirable side-effects of the small molecule inducer, for example, the tet system is induced by tetracycline which can inhibit cell growth. Finally, whilst the common expression systems are up-regulated upon addition of small molecule ligands, there are few systems available where addition of ligand represses expression of the gene of interest. In generating conditional mutants, it is desirable for the essential gene of interest to be expressed at first, to allow growth to be established, and then down-regulated at the desired time point upon the addition of an effector molecule. Whilst some of these issues can be overcome by engineering improved host strains, this can be laborious. For example, it has taken several decades to engineer E. coli host strains that are compatible with the many different expression systems that are now commercially available. As a result, there are many bacterial species for which common expression systems are incompatible and where there are few or no alternative expression systems available. This is particularly relevant for pathogenic strains, where expression tools are crucial in the effort to develop new antimicrobial treatments.

Alternative methods have been sought to control gene expression with small molecules. For example, aptamers, generated by in vitro selection (e.g. SELEX), have been inserted into the 5′-untranslated regions (5′-UTRs) of bacterial mRNA to control translation in response to selected ligands. Notably, a theophylline aptamer inserted in the 5′-UTR of a β-galactosidase (lacZ) reporter was shown to sequester the ribosome binding site (rbs) in the absence of its ligand (Lynch et al. Chem Biol 2007, 14, 173). Upon binding theophylline, the rbs is released from the aptamer and translation is initiated. Whilst aptamers have significant potential for small molecule modulation of gene expression, there are currently very few aptamers available that are selective for ligands with desirable physiochemical and pharmacokinetic properties. Indeed, to date, only theophylline and a recently engineered atrazine aptamer have been shown to be effective at controlling gene expression in bacteria (Sinha et al. Nature Chem. Biol. 2010, 6, 464). However, nature has already evolved its own aptamers that provide exquisite control of gene expression through binding to a wide range of small molecule metabolites including amino acids, nucleobases, carbohydrates and co-enzymes. These so-called riboswitches are found in all domains of life, occurring with highest frequency in bacteria where they usually regulate genes involved in metabolite biosynthesis, catabolism, or transport. The bacterial riboswitches typically function through the binding of a metabolite to an aptamer domain, within the 5′-UTR of mRNA, which affects the conformation of an adjacent expression platform, thus modulating either the termination of transcription, initiation of translation or mRNA self-cleavage. This is a fundamentally different paradigm from protein-based regulatory systems. Riboswitches therefore offer an ideal platform to develop small-molecule controllable expression systems which address many of the limitations of the classical systems. However, the applicability of riboswitches as generic genetic control elements is limited by the fact that natural riboswitches have evolved to bind to primary metabolites that are common within cells. The aptamer domain within each category of riboswitch is generally highly conserved, and as such they can be identified readily using homology based searches. In contrast, the expression platform of a riboswitch often varies in sequence and structure, and controls the expression of the coding sequence to which it is operably linked. For example, the TPP riboswitch in bacteria shares a highly conserved aptamer domain to which the ligand binds, but in different bacterial strains the expression platform varies: in E. coli it causes inhibition of translation by blocking the Shine-Dalgarno sequence, whereas in B. subtilis it causes termination of transcription.

The effects of a riboswitch on gene expression include transcriptional termination, inhibition of translation initiation, mRNA self-cleavage, and in eukaryotes, alteration of splicing pathways. Specifically, the following mechanisms of action have been observed: riboswitch-controlled formation of Rho-independent transcription termination hairpins, leading to premature transcription termination; riboswitch-mediated folding resulting in sequestration of the ribosome-binding site, thereby inhibiting translation; dual role of the riboswitch as a control mechanism and a ribozyme, the latter being cleaved in the presence of sufficient concentrations of ligand; and alternative structures of the riboswitch affecting the splicing of the pre-mRNA.

The mechanisms of action of the riboswitches can have higher levels of complexity. For example, a riboswitch in Clostridium acetobutylicum has been shown to regulate not the gene which it is linked to, but an adjacent gene which is not part of the same mRNA transcript. Another similar example has been observed in Listeria monocytogenes, where the riboswitch regulates the expression of a downstream gene. However, riboswitch-controlled transcripts can also subsequently modulate the expression of genes located elsewhere in the genome.

Known riboswitches are generally categorised by the ligand which binds them. Riboswitches discovered to date include:
Cobalamin riboswitch (also B₁₂-element), which binds adenosylcobalamin (the coenzyme form of vitamin B₁₂) to regulate the biosynthesis and transport of cobalamin and similar metabolites.
cyclic di-GMP riboswitches, which bind cyclic di-GMP to regulate a variety of genes. Two non-structurally related classes exist—cyclic di-GMP-I and cyclic di-GMP-II.
FMN riboswitch (also RFN-element) which binds flavin mononucleotide (FMN) to regulate riboflavin biosynthesis and transport.
glmS riboswitch, which cleaves itself when there is a sufficient concentration of glucosamine-6-phosphate.
Glutamine riboswitches, which bind glutamine to regulate genes involved in glutamine and nitrogen metabolism. They also bind short peptides of unknown function. Such riboswitches fall into two classes, which are structurally related: the glnA RNA motif and Downstream-peptide motif.
Glycine riboswitch, which binds glycine to regulate glycine metabolism genes. It comprises two adjacent aptamer domains in the same mRNA, and is the only known natural RNA that exhibits cooperative binding.
Lysine riboswitch (also L-box), which binds lysine to regulate lysine biosynthesis, catabolism and transport.
PreQ1 riboswitches, which bind pre-queuosine₁, to regulate genes involved in the synthesis or transport of this precursor to queuosine. Two entirely distinct classes of PreQ1 riboswitches are known: PreQ1-I riboswitches and PreQ1-II riboswitches. The binding domain of PreQ1-I riboswitches is unusually small among naturally occurring riboswitches. PreQ1-II riboswitches, which are only found in certain species in the genera Streptococcus and Lactococcus, have a completely different structure, and are larger.
Purine riboswitches, which bind purines to regulate purine metabolism and transport. Different forms of the purine riboswitch bind guanine (a form originally known as the G-box) or adenine. The specificity for either guanine or adenine depends completely upon Watson-Crick interactions with a single pyrimidine in the riboswitch at position Y74. In the guanine riboswitch this residue is always a cytosine (i.e. C74), in the adenine residue it is always a uracil (i.e. U74). Homologous types of purine riboswitches bind deoxyguanosine, but have more significant differences than a single nucleotide mutation.
SAH riboswitches, which bind S-adenosylhomocysteine to regulate genes involved in recycling this metabolite which is produced when S-adenosylmethionine is used in methylation reactions.
SAM riboswitches, which bind S-adenosyl methionine (SAM) to regulate methionine and SAM biosynthesis and transport. Three distinct SAM riboswitches are known: SAM-I (originally called S-box), SAM-II and the S_{MK} box riboswitch. SAM-I is widespread in bacteria, but SAM-II is found only in α-, β- and a few γ-proteobacteria. The S_{MK} box riboswitch is found only in the order Lactobacillales. These three varieties of riboswitch have no obvious similarities in terms of sequence or structure. A fourth variety, SAM-IV, appears to have a similar ligand-binding core to that of SAM-I, but in the context of a distinct scaffold.
SAM-SAH riboswitches, which bind both SAM and SAH with similar affinities. Since they are always found in a position to regulate genes encoding methionine adenosyltransferase, it was proposed that only their binding to SAM is physiologically relevant.
Tetrahydrofolate riboswitches, which bind tetrahydrofolate to regulate synthesis and transport genes.
TPP riboswitches (also THI-box), which bind thiamine pyrophosphate (TPP) to regulate thiamine biosynthesis and transport, as well as transport of similar metabolites. It is the only riboswitch found so far in eukaryotes.
Moco riboswitch, which binds molybdenum cofactor, to regulate genes involved in biosynthesis and transport of this coenzyme, as well as enzymes that use it or its derivatives as a cofactor.
Adenine sensing add-A riboswitch, found in the 5′ UTR of the adenine deaminase encoding gene (add) of Vibrio vulnificus.

The present invention aims to provide novel genetic systems to regulate gene expression, using riboswitches as a regulatory tool. Such genetic systems, genetic constructs, and kits or methods utilising same, may be of use in controlling gene expression, for example in the production of recombinant proteins.

The invention also aims to provide novel ligands for riboswitches. Preferably, a non-natural ligand for a riboswitch will be one which is generally not present in a cell, preferably is drug-like, preferably can penetrate the cell membrane, preferably is non-toxic, preferably is stable, cheap and preferably shows high specificity.

Riboswitches represent a target for therapeutic intervention, particularly for the development of novel pharmaceuticals and anti-bacterial agents. Riboswitch-regulated systems of the invention may also be suitable for use in therapeutic indications. Systems or constructs for use in such applications will preferably be transferable, allowing simultaneous yet differential control of multiple genes. Preferably, it will be capable of activating and blocking gene expression, be capable of acting rapidly and in a dose-dependent manner, and/or have broad dynamic range and temporal control.

### BRIEF SUMMARY OF THE DISCLOSURE

Thus, in a first aspect of the invention, there is provided a genetic construct, comprising a first nucleic acid molecule encoding a riboswitch and a second nucleic acid molecule comprising a coding sequence, wherein the first and second nucleic acid molecules are operably linked to one another by a third nucleic acid molecule comprising a spacer sequence.

Preferably, the spacer is a nucleic acid molecule of low GC content, preferably less than 65%, 60%, 55%, 50%, 55%, 50%, 45%, 40%, 39%, 38%, 37%, 36%, 35%, 34%, 33%, 32%, 31%, 30%, 29%, 28%, 27%, 26%, 25%, 24%, 23%, 22%, 20%, 19%, 18%, 17%, 16%, 15%, 14%, 13%, 12%, 11%, 10%, 9%, 8%, 7%, 6%, 5%, 4%, 3%, 2% or 1%, across the full length of the spacer, or across at least 50%, 60%, 70%, 80%, 90%, 91%, 92%, 93%, 94%, 95%, 96%, 97%, 98% or 99% contiguous nucleic acid residues of the spacer. Preferably, the spacer is substantially free of secondary structure, preferably comprising less than 40 kcal/mol, more preferably less than -39, -38, -37, -36, -35, -34, -33, -32, -31, -30, -29, -28, -27, -26, -25, -24, -23, -22, -20, -19, -18, -17, -16, -15, -14, -13, -12, -11, -10, -9, -8, -7, -6, -5, -4, -3, -2 or -1 kcal/mol. Preferably, the spacer is a nucleic acid, preferably DNA or RNA.

The spacer sequence may encode an RNA sequence, and preferably a protein or peptide sequence, including a secretion signal peptide.

The nucleic acid molecules of the genetic construct of the first aspect may be DNA molecules, but more preferably are RNA molecules. Thus, the first aspect of the present invention also provides a genetic construct comprising a first RNA molecule comprising a riboswitch and a second RNA molecule comprising a coding sequence, wherein the first and second RNA molecules are operably linked by a third RNA molecule comprising a spacer sequence.

Within the genetic construct, preferably, the first nucleic acid molecule is upstream of the third nucleic acid molecule, and the third nucleic acid molecule in turn upstream of the second nucleic acid molecule.

Preferably, in the first aspect of the invention, two or more of the first, second and third nucleic acid molecules of the genetic construct are operably linked to each other. More preferably, they are structurally linked. More preferably, they are linked by nucleic acid residues. More preferably, they are contiguous, such that the last nucleic acid residue of one nucleic acid molecule is adjacent in sequence to the last nucleic acid residue of another of the first, second or third nucleic acid molecules.

Two or more of the first, second and third nucleic acid molecules may each be contiguous. Alternatively, two or more of the first, second or third nucleic acid molecules may be indirectly linked by intervening sequence. Preferably, they are operably linked. Preferably, at least the third nucleic acid molecule comprising the spacer sequence and the second nucleic acid molecule comprising the coding sequence are contiguous. More preferably, the last nucleic acid residue of the spacer directly linked to, the first nucleic acid residue of the coding sequence. Where the spacer is a non-coding sequence, a start codon may be provided in the coding sequence of the second molecule. In this case, it is envisaged that the first nucleic acid residue of the coding sequence of the second nucleic acid molecule may be the A residue of a start codon, such as AUG. Where the spacer encodes an RNA or protein or peptide sequence, a start codon may be provided in the spacer sequence. In embodiments where the third molecule is itself a coding sequence (as considered elsewhere in the present invention) the third molecule should be in-frame with the second molecule, such that an extended mRNA corresponding to both the second and third molecules is transcribed. In such cases a N-terminally tagged protein and/or mini-fusion protein may be expressed.

It is also envisaged that an intervening sequence may be provided between the third nucleic acid molecule and the second nucleic acid molecule, for example a nucleic acid molecule comprising a second riboswitch. Such a second riboswitch may be the same or different to the first riboswitch, as defined herein.

Such a genetic construct may comprise, in the following order:

RIBOSWITCH-SPACER-INTERVENING SEQ (e.g. RIBOSWITCH)-CODING SEQUENCE.

In this embodiment, preferably the last nucleic acid residue of the spacer sequence of the third nucleic acid molecule is contiguous with the intervening sequence. Preferably, the last nucleic acid residue of the intervening molecule is contiguous with the last nucleic acid residue of the second nucleic acid molecule, preferably to the first nucleic acid residue of the coding sequence.

Preferred genetic constructs of the first aspect comprise an orthogonal riboswitch (herein referred to as “ORS”). Preferred riboswitches for use in the present invention include the M6″ and addA riboswitches, preferably operably linked to sequence encoding a T7 polymerase. Preferably, the riboswitch-T7 polymerase sequence is under the control of a lac promoter (though it will be appreciated that other promoters considered elsewhere in the specification may also be used). Preferably, the genetic construct also comprises a kanamycin and an ampicillin resistance marker genes. Preferred genetic constructs of the first aspect of the invention are shown in FIGS. 1, 2, 23, 24, 26 (pMOD3_spT7, operon, dual promoter and ORS_sT7), 41, 42, 43, 44, 45.

The first aspect of the invention also provides a kit of parts, comprising a genetic construct as herein described, and a ligand. Preferably, the ligand is a ligand of the fourth aspect.

The first aspect of the invention also provides a method of regulating expression of a coding sequence, the method comprising: contacting a cell or expression system comprising a genetic construct of the invention with a ligand capable of activating a riboswitch of the genetic construct. Preferably, the method is selected from a method of inhibiting bacterial cell growth, a method of increasing or decreasing the yield of a coding sequence product or products, a method of altering the physiological characteristics of a cell, a method of increasing gene production, a method altering cell morphology, a method of altering sensitivity to an antibiotic, use as a tool for drug discovery, use as a biosensor, or a method of increasing flux through a biosynthetic pathway. Preferably, the ligand is a ligand of the fourth aspect.

The first aspect of the invention also provides a cell or expression system comprising a genetic construct of the first aspect, and optionally a ligand capable of activating a riboswitch of the genetic construct of the first aspect. Preferably, the ligand is a ligand of the fourth aspect.

### Decoupled Riboswitch and Gene of Interest

In a second aspect of the invention, there is provided a system comprising
a) a first genetic construct comprising a first nucleic acid molecule encoding a riboswitch operably linked to a second nucleic acid molecule comprising a regulatory sequence; and optionally
b) a second genetic construct comprising a nucleic acid molecule comprising a coding sequence whose expression is capable of being regulated by a gene product of the second nucleic acid molecule.

The regulatory sequence of the second nucleic acid molecule may encode a polymerase, a transcription factor, an oscillator, a specificity factor, a repressor, an activator, or a regulatory RNA molecule, which serves to regulate expression of the coding sequence of the second genetic construct. Thus, the riboswitch acts indirectly on the coding sequence of the second genetic construct by regulating the expression of a component which effects the expression of the coding sequence. Alternatively, the regulatory sequence may encode an antisense sequence, which is targeted against a component which effects expression of the coding sequence of the second construct, for example a polymerase, a transcription factor, an oscillator, a specificity factor, a repressor, an activator, or a regulatory RNA molecule. Where the regulatory molecule encodes polymerase, this may be any suitable polymerase, preferably a viral polymerase, and preferably T7, SP6, or T3 viral polymerase (pMLOST, and pMAST3).

Preferably, the first and second genetic constructs may independently be DNA or RNA. Preferably, the nucleic acid molecules are independently DNA molecules. Thus, the second aspect of the present invention also provides

a) a first genetic construct comprising a first RNA molecule comprising a riboswitch operably linked to a second RNA molecule which is, or encodes, a regulatory sequence; and optionally
b) a second genetic construct comprising nucleic acid molecule comprising a coding sequence, whose expression is capable of being controlled by the regulatory sequence or a gene product of the regulatory sequence of the first construct.

The nucleic acid molecule of the second construct may be DNA or RNA independently of the nature of the nucleic acid molecules of the first construct.

Preferably, where the regulatory sequence encodes an antisense sequence, this may regulate the translation of the RNA molecule encoded by the second construct, decrease the half life of the target, or target the RNA for nuclease cleavage. See, for example, the sequences of FIGS. 17 and 33, which represent preferred embodiments of the invention.

Thus, where the regulatory sequence encodes a polymerase, the regulated expression of the polymerase in turn affects the expression of the coding sequence of the second genetic construct, which is transcribed by the polymerase of the first genetic construct. Thus, the expression of the coding sequence of the second construct is dependent upon the expression of the polymerase, for example in terms of the level of polymerase or its temporal expression.

Preferably, in the second aspect of the invention, in the first genetic construct the first and second nucleic acid molecules are operably linked to each other. More preferably, they are structurally linked. More preferably, they are contiguous.

Within the first genetic construct, preferably the first nucleic acid molecule is provided upstream of the second nucleic acid molecule.

In a preferred embodiment of the second aspect, the first genetic construct additionally comprises a nucleic acid molecule comprising a spacer sequence. The nucleic acid molecule comprising the spacer sequence is preferably provided upstream of the nucleic acid molecule comprising a regulatory sequence. The nucleic acid molecule comprising the spacer sequence may be operably linked to the nucleic acid molecule comprising the regulatory sequence, either contiguously or indirectly. Contiguous is preferred, as defined herein. Where the spacer is a non-coding sequence, a start codon may be provided in the coding sequence of the second molecule. In this case, it is envisaged that the first nucleic acid residue of the coding sequence of the second nucleic acid molecule may be the A residue of a start codon, such as AUG. Where the spacer encodes an RNA or protein or peptide sequence, a start codon may be provided in the spacer sequence.

Thus, for example, the first genetic construct of the second aspect of the invention may comprise:

### RIBOSWITCH-(OPTIONAL SPACER)-REGULATORY SEQUENCE

In a preferred embodiment of the second aspect, the second genetic construct may independently comprise a nucleic acid molecule comprising a spacer sequence. The nucleic acid molecule comprising the spacer sequence is preferably provided upstream of the nucleic acid molecule comprising the coding sequence. The nucleic acid molecule comprising the spacer sequence may be operably linked to the nucleic acid molecule comprising the coding sequence, either contiguously or indirectly. Contiguous is preferred, as defined herein. More preferably, the last nucleic acid residue of the nucleic acid molecule comprising the spacer sequence is adjacent to the first nucleic acid residue of the nucleic acid molecule comprising the coding sequence. For example, the last nucleic acid residue of the spacer is immediately upstream, or adjacent to, the first nucleic acid residue of the coding sequence. Where the spacer is a non-coding sequence, a start codon may be provided in the coding sequence of the second molecule. In this case it is envisaged that the first nucleic acid residue of the coding sequence of the nucleic acid molecule may be the A residue of a start, for example AUG, for example where the spacer is a non-coding sequence. Where the spacer encodes an RNA or protein or peptide sequence, a start codon may be provided in the spacer sequence.

The second aspect of the invention also provides a kit of parts, comprising a first genetic construct and optionally a second genetic construct of the second aspect of the invention, and optionally a ligand of the riboswitch of the first genetic construct. Preferably, the ligand is a ligand of the fourth aspect.

The second aspect of the invention also provides a method of regulating expression of a coding sequence, the method comprising: contacting a cell or expression system comprising a first and optionally second genetic construct of the second aspect of the invention with a ligand of the riboswitch. Preferably, the method may be a method of inhibiting bacterial cell growth, a method of increasing or decreasing the yield of a coding sequence product or multiple products, a method of altering the physiological characteristics of a cell, a method of increasing gene production, a method altering cell morphology, a method of altering sensitivity to an antibiotic, use as a tool for drug discovery, use as a biosensor, or a method of increasing flux through a biosynthetic pathway. Preferably, the ligand is a ligand of the fourth aspect.

The second aspect of the invention also provides a cell or expression system comprising a first genetic construct, optionally a second genetic construct of the second aspect, and optionally a ligand of the riboswitch of the first genetic construct. Preferably, the ligand is a ligand of the fourth aspect.

### Multiple Riboswitches Controlling Multiple Genes

In a third aspect of the invention, there is provided a genetic construct comprising
a) a first nucleic acid molecule encoding a riboswitch, the first nucleic acid molecule being operably linked to a second nucleic acid molecule comprising a coding sequence; and
b) a third nucleic acid molecule encoding a second riboswitch, the third nucleic acid molecule being operably linked to a fourth nucleic acid molecule comprising a second coding sequence.

Optionally, the genetic construct may comprise further (3, 4, 5, 6 or more) nucleic acid molecules, each encoding a further riboswitch, and each being independently and operably linked to a further (2, 3, 4, 5, 6 or more) nucleic acid molecule comprising a coding sequence. This aspect of the invention allows variable co-expression of any multimeric or multi-domain protein or complex, such as heavy and light chain antibodies, or any ancillary protein e.g. chaperone, secretion protein or glycosylation proteins.

Preferably, the genetic construct comprises a transcriptional terminator between a coding sequence and a downstream second or further riboswitch. Thus, for example, a transcriptional terminator may be provided between the second nucleic acid molecule and the third nucleic acid molecule or between a fourth nucleic acid molecule and any fifth or further nucleic acid molecules. In any genetic construct, one or more transcriptional terminators may be provided, as appropriate.

The order of components in a genetic construct of the third aspect may be:

### PROMOTER-RIBOSWITCH-CODING SEQUENCE-TRANSCRIPTIONAL TERMINATOR-PROMOTOR 2-RIBOSWITCH 2-CODING SEQUENCE 2

The presence of a transcriptional terminator enables two or more RNA molecules to be yielded.

### RIBOSWITCH 1-CODING SEQUENCE 1 and RIBOSWITCH 2-CODING SQUENCE 2

Such a configuration is shown as a preferred embodiment of the invention in FIG. 42. In such an embodiment, a genetic construct is provided which comprises an M6″ riboswitch upstream of and operably linked to a T7RNAP coding sequence, and downstream thereof an AddA riboswitch upstream of and operably linked to a T3RNAP coding sequence. Both pairings are under the control of a lac promoter. Riboswitch mediated control of expression of the T7 and T3 polymerases serves to regulate, for example, expression of domains of a multimeric protein, the expression each domain or protein being under the control of a T3 or T7 promoter. This aspect of the invention is also useful in achieving variable co-expression of coding sequence products (such as pTTv7 and pTTv9, as described elsewhere in the specification).

Where there is no transcriptional terminator provided between a coding sequence and a downstream riboswitch, two or more riboswitches and coding sequences may be provided within a single RNA molecule, i.e.

### RIBOSWITCH-CODING SEQUENCE 1-RIBOSWITCH2-CODING SEQUENCE2

Preferably, all genetic molecules within a genetic construct will be of the same type, ie DNA or RNA. Thus, the third aspect of the present invention also provides a genetic construct comprising a first RNA molecule comprising a riboswitch and a second RNA molecule comprising a coding sequence, and a third RNA molecule encoding a second riboswitch, the third RNA molecule being operably linked to a fourth RNA molecule comprising a second coding sequence.

Two or more riboswitches in the genetic construct of the third aspect of the invention may be the same or may be different. Optionally, two or more may be the same, and the remainder may each be the same or different thereto.

The coding sequence of the genetic constructs may be regulatory sequences, as defined herein, or may encode a non-regulatory RNA or protein or peptide sequence. Where regulatory, the product of a coding sequence may in turn control the expression of a further coding sequence within the construct, or within an additional genetic construct.

The coding sequences of the two or more nucleic acid molecules comprising the same may be the same or different. Two or more of the coding sequences may be the same, and the remainder may each be the same or different thereto. By the same is meant that they encode substantially the same product (RNA or protein or peptide), this being measured in terms of primary sequence, secondary structure and/or function.

Preferably, in the third aspect of the invention, in the genetic construct the first and second nucleic acid molecules are operably linked to each other, and the third and fourth nucleic acid molecules are operably linked to each other. More preferably, the first, second, third and fourth nucleic acid molecules are operably linked to one another. More preferably, the nucleic acid molecules which are operably linked may be structurally linked. More preferably, they are linked by nucleic acid residues. More preferably, they are contiguous.

Within the genetic construct, the first and second nucleic acid molecules may be provided in any order. However, preferably, the first nucleic acid molecule is upstream of the second or further nucleic acid molecules, and in turn the second is upstream of the third and so on.

Preferably, one or more nucleic acid molecules of the genetic construct may each independently additionally comprise a spacer sequence. Preferably, at least one nucleic acid molecule comprising a coding sequence also comprises a spacer sequence. Preferably, two or more nucleic acid molecules comprising a coding sequence may each independently comprise a spacer sequence. Preferably, each nucleic acid molecule comprising a riboswitch also comprises a spacer sequence. Where two or more nucleic acid molecules comprising spacer sequences are provided in the genetic construct of the third aspect, these spacer sequences may each be the same or different.

Two or more may be the same, and the remainder may each, independently, be the same or different thereto.

A spacer sequence may be operably linked to a nucleic acid molecule comprising a coding sequence, either contiguously or indirectly linked. Contiguous is preferred, as defined herein. More preferably the last nucleic acid residue of the riboswitch is immediately upstream, or adjacent to, the first nucleic acid residue of the riboswitch. Where the spacer is a non-coding sequence, a start codon may be provided in the coding sequence of the second molecule. In this case, it is envisaged that the first nucleic acid residue of the coding sequence of the second nucleic acid molecule may be the A residue of a start codon, such as AUG. Where the spacer encodes an RNA or protein or peptide sequence, a start codon may be provided in the spacer sequence, allowing expression of an expression tag and/or mini-fusion protein.

A preferred genetic construct of the third aspect comprises an M6″ riboswitch operably linked to a DsRed coding sequence, and an AddA riboswitch operably linked to a eGFP coding sequence. Preferably, the AddA-GFP sequence is downstream of the M6″-DsRed sequence. Preferably, the genetic construct is pOperon, of FIG. 2. An alternative embodiment comprises a transcription termination sequence downstream of the M6″-DsRed sequence, and upstream of the AddA-GFP sequence. Preferably, the genetic construct is pDual Promoter of FIG. 2.

The third aspect of the invention also provides a kit of parts, comprising a genetic construct of the third aspect of the invention, and a ligand of a riboswitch of the construct. Preferably, a ligand is a ligand of the fourth aspect. The kit may also comprise additional ligands for additional riboswitches.

The third aspect of the invention also provides a method of regulating expression of a coding sequence, the method comprising: contacting a cell or expression system comprising a genetic construct of the third aspect of the invention with a ligand of a riboswitch of the construct. Preferably, the method may be a method of inhibiting bacterial cell growth, a method of increasing or decreasing the yield of a coding sequence product or multiple products a method of altering the physiological characteristics of a cell, a method of increasing gene production, a method altering cell morphology, a method of altering sensitivity to an antibiotic, use as a tool for drug discovery, use as a biosensor, or a method of increasing flux through a biosynthetic pathway. Preferably, the ligand is a ligand of the fourth aspect.

The third aspect of the invention also provides a cell or expression system comprising a genetic construct of the third aspect, and optionally a ligand of a riboswitch of the genetic construct. Preferably, the ligand is a ligand of the fourth aspect.

### Ligands

In a fourth aspect of the invention, there is provided a ligand defined by Formula I;

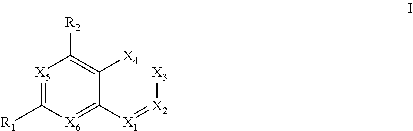

wherein
R₁ and R₂ are independently selected from a hydrogen bond donor or a hydrogen bond acceptor, for example H or NH₂, O, OH; and
X₁, X₂, X₃, X₄ X₅, and X₆ are independently selected from any hydrogen bond donor or hydrogen bond acceptor, or from CH or N.

In a preferred embodiment, R₁ and R₂ are independently selected from O, OH, NH₂, NHCOCH₃, NHCO₂CH₃, NHcCH(CH₂CH₂), SH, NHNHCOCH₃, NHOCH₃, or NHOR (R=alkyl, aryl, or benzyl).

In a preferred embodiment, the compound of Formula I is

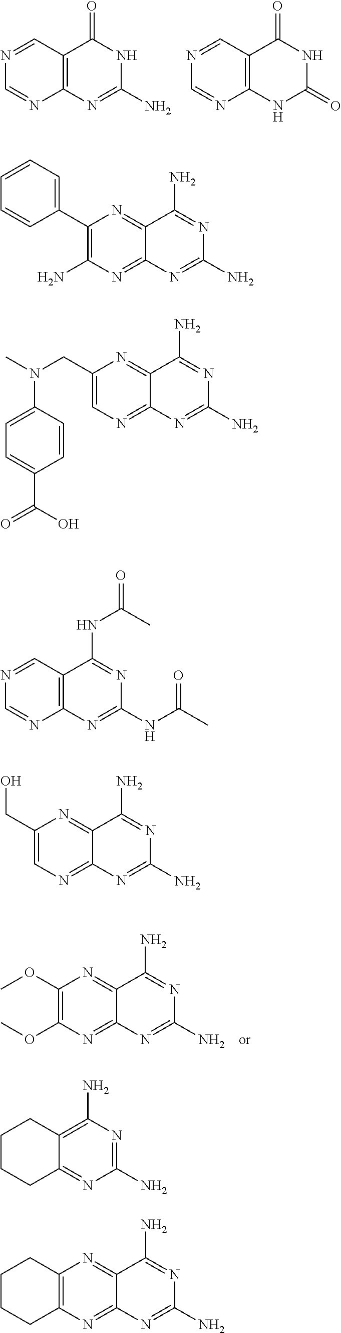

In a preferred embodiment, both R₁ and R₂ are NH₂.

In a preferred embodiment, the compound of Formula I is

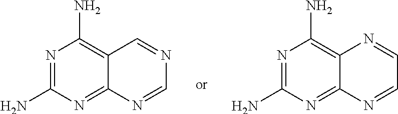 [SMILES: Nc1nc(N)c2cncnc2n1 ; Nc1nc(N)c2nccnc2n1]

Also provided by the fourth aspect of the invention is a ligand defined by Formula II

### II

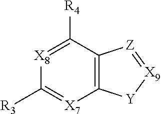

wherein
R₃ and R₄ are independently selected from a hydrogen bond donor or a hydrogen acceptor;
X₇, X₈ and X₉ are independently selected from CH or any hydrogen bond donor or any hydrogen bond acceptor.
Y is independently selected from N, O, S, NR, where R is independently selected from alkyl, ribose, deoxyribose, dideoxyribose, (hydroxymethyl)morpholino analogues of ribose or 5′-modified ribose;
Z is independently selected from any one of N, C attached to —CH₂OH, —CH₂NH₂, CN, or CONH₂

In a preferred embodiment, Z is O.

In a preferred embodiment, both R₃ and R₄ are NH₂.

In a preferred embodiment, the compound of Formula II is:

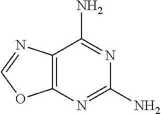 [SMILES: Nc1nc(N)c2ncoc2n1]

In a preferred embodiment, R₃ and R₄ are independently selected from OH, NH₂, NHCOCH₃, NHCO₂CH₃, NHcCH(CH₂CH₂), SH, NHNHCOCH₃, NHOCH₃, NHCO CH(CH₂)₃ NHOR (where R=alkyl, aryl, or benzyl).

In a preferred embodiment Formula II may be a compound of formula:

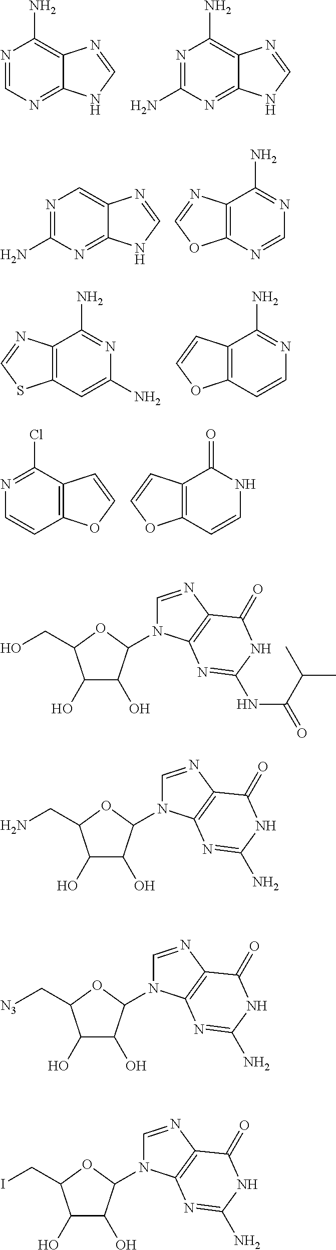

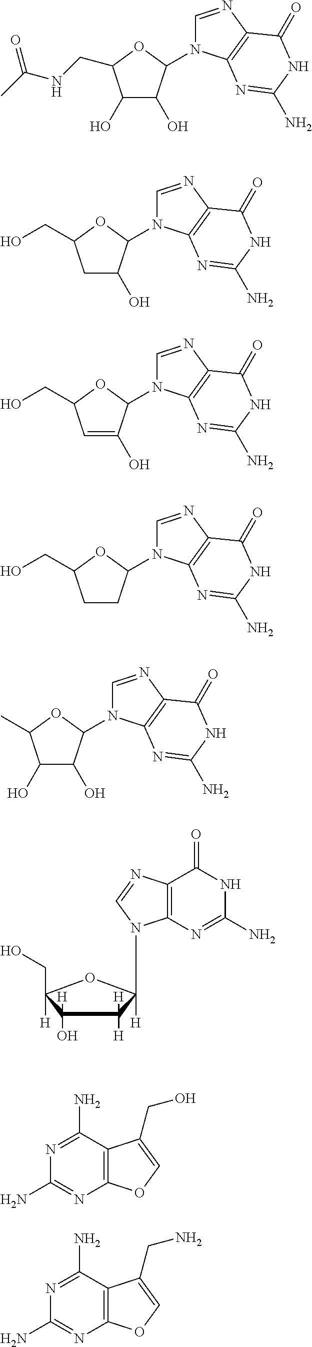

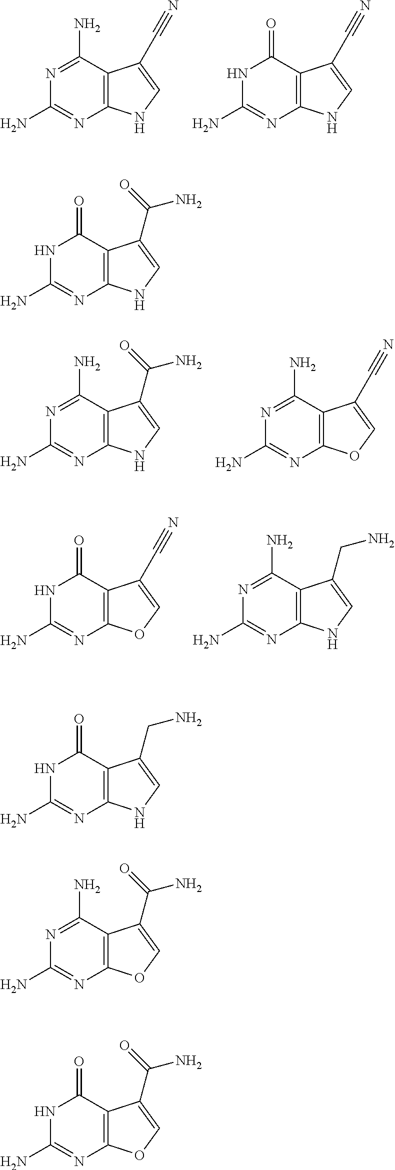

Also provided by the fourth aspect of the invention is a ligand defined by Formula III

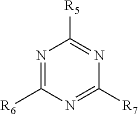

wherein
R₅, R₆ and R₇ is independently selected from aryl or heteroaryl; and R₅ is optionally substituted by C(O)NH₂, are independently selected from H, NH₂, O, OH, NHCOCH₃, NHCO₂CH₃, NHcCH(CH₂CH₂), SH, NHNHCOCH₃, NHOCH₃, NHCO CH(CH₂)₃ NHOR (where R=alkyl, aryl, or benzyl).

In a preferred embodiment, the compound of Formula III may be selected from:

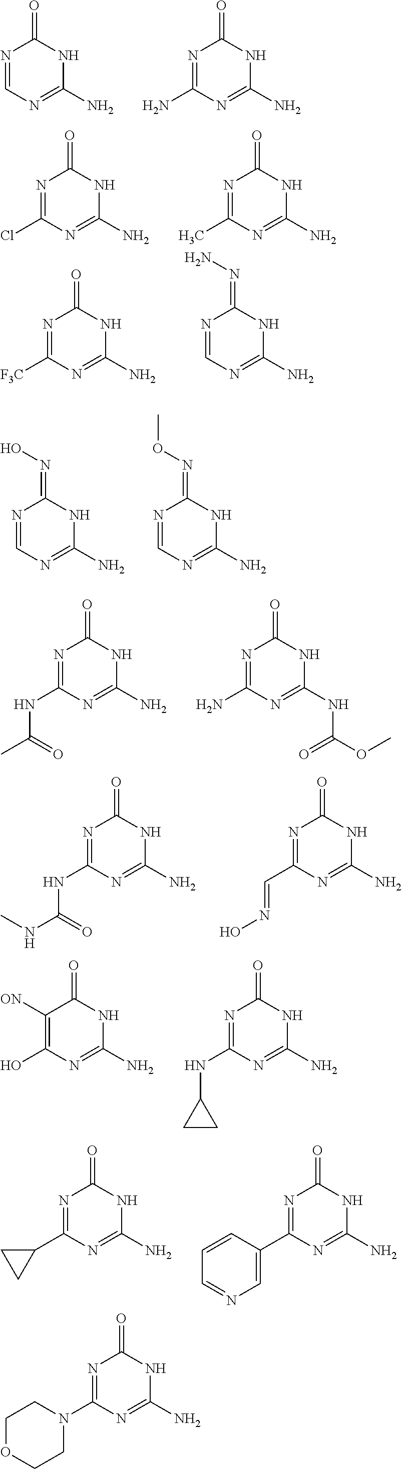

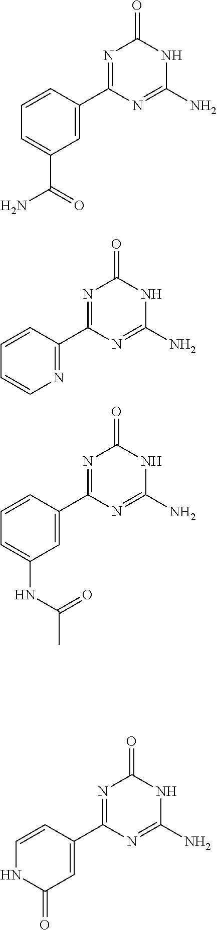 [SMILES: NC(=O)c1cccc(-c2nc(N)[nH]c(=O)n2)c1 ; Nc1nc(-c2ccccn2)nc(=O)[nH]1 ; CC(=O)Nc1cccc(-c2nc(N)[nH]c(=O)n2)c1 ; Nc1nc(-c2cc[nH]c(=O)c2)nc(=O)[nH]1]

Suitably, a ligand for use in the present invention is a compound of any of Formulas I, II or III as herein defined.

Depending upon the groups that are selected, a first group can be incorporated within second group or, alternatively, the first group can be pendant (i.e. attached) to the second group. For example, with the phrase “an alkyl group comprising an amino group”, the amino group can be attached to the backbone of the alkyl group. The nature of the group(s) that is (are) selected with determine if the first group is embedded or attached to the second group.

Unless stated to the contrary, a formula with chemical bonds shown only as solid lines are not as wedges or dashed lines contemplates each possible isomer, e.g. each enantiomer and diastereomer and a mixture of isomers such as a racemic or scalemic mixture.

A ligand of Formula I, II or III may be used in combination with any of the genetic constructs of the first or second aspects of the invention, as a system or kit to regulate gene expression.

Preferably, in systems and kits comprising a ligand of Formula I, II or III of the fourth aspect, a riboswitch provided in the genetic construct is preferably a purine riboswitch (Kim Biol Cell 100:1 (2008)), an Add-A riboswitch, preferably a modified Add-A riboswitch, preferably a modified Add-A riboswitch known as M6, M6′, M6″ or M6C, M6C′ or M6C″ (Dixon et al PNAS 107: 2830 (2010)), or a PreQ switch or a modified PreQ switch, for example the PreQ switch of FIG. 14 or a riboswitch which is derived therefrom.

Alternatively, the riboswitch may be a chimeric or engineered riboswitch.

Thus, a ligand of the fourth aspect may preferably be used in combination with a genetic construct comprising a riboswitch which comprises a ligand binding site of a purine riboswitch such as that of FIG. 11, or one which is derived therefrom, preferably having at least 50, 60, 70, 80, 85, 90, 95, 96, 99, 98 or 99% sequence identity thereto and preferably being capable of binding a ligand of Formula I, II or III and causing a conformational change in the expression platform of the riboswitch. Preferably, the conformational change is sufficient to cause a change in expression of a coding sequence operably linked to the riboswitch. Preferably, the ligand will be one of Formula I, II or III.

Thus, a ligand of the fourth aspect may preferably be used in combination with a genetic construct comprising a riboswitch which comprises a ligand binding site of an Add-A riboswitch such as that of FIG. 13, or one which is derived therefrom, such as the M6, M6′, M6″ or M6C, M6C′ or M6C″ (Dixon et al PNAS 107: 2830 (2010)). The riboswitch may be one having at least 50, 60, 70, 80, 85, 90, 95, 96, 99, 98 or 99% sequence identity thereto and preferably being capable of binding a ligand of Formula I, II or III and causing a conformational change in the expression platform of the riboswitch. Preferably, the conformational change is sufficient to cause a change in expression of a coding sequence operably linked to the riboswitch. Preferably, the ligand will be one of Formula I, II or III.

A ligand of the fourth aspect may also be used in combination with a genetic construct comprising a riboswitch which comprises a ligand binding site of a PreQ riboswitch such as that of FIG. 14, or one which is derived therefrom, preferably having at least 50, 60, 70, 80, 85, 90, 95, 96, 99, 98 or 99% sequence identity thereto and preferably being capable of binding a ligand of Formula II and causing a conformational change in the expression platform of the riboswitch. Preferably, the conformational change is sufficient to cause a change in expression of a coding sequence operably linked to the riboswitch. Preferably, the ligand will be one of Formula I, II or III.

Preferred ligands falling within the scope of Formula I, II or III are those having a K_{d} of below 10 μM, preferably below 9, preferably below 8, 7, 6, 5, 4, 3, 2 or 1.

### BRIEF DESCRIPTION OF THE DRAWINGS

Embodiments of the invention are further described hereinafter with reference to the accompanying drawings, in which:

FIG. 1 is a diagrammatic illustration of examples of genetic constructs, comprising Origin of replication (ORI) Orthogonal riboswitch (ORS), T7 RNA polymerase (T7pol), T7 promoter (T7pro), Multi-cloning site (MCS), lac promoter (lacP), chromosomal insertion site (CIS), Zeocin antibiotic resistance marker (Zeo);

FIG. 2 is a diagrammatic illustration of further genetic constructs, comprising Orthogonal riboswitch (ORS), T7 RNA polymerase (T7), spacer T7 fusion protein (spT7). Lac promoter (lacP), and transcription terminator (term);

FIG. 3 shows the ligand effects on eGFP expression regulated by lac promoter-operator and AddA riboswitch;

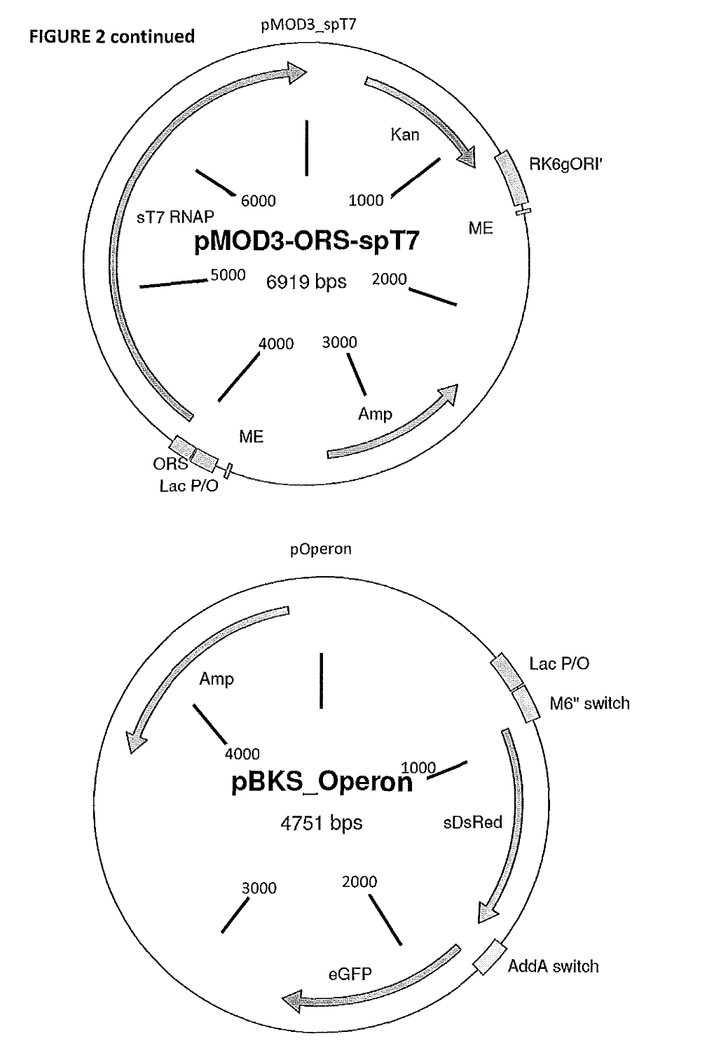

FIG. 4 shows ligand effects on eGFP expression regulated by AddA riboswitch;

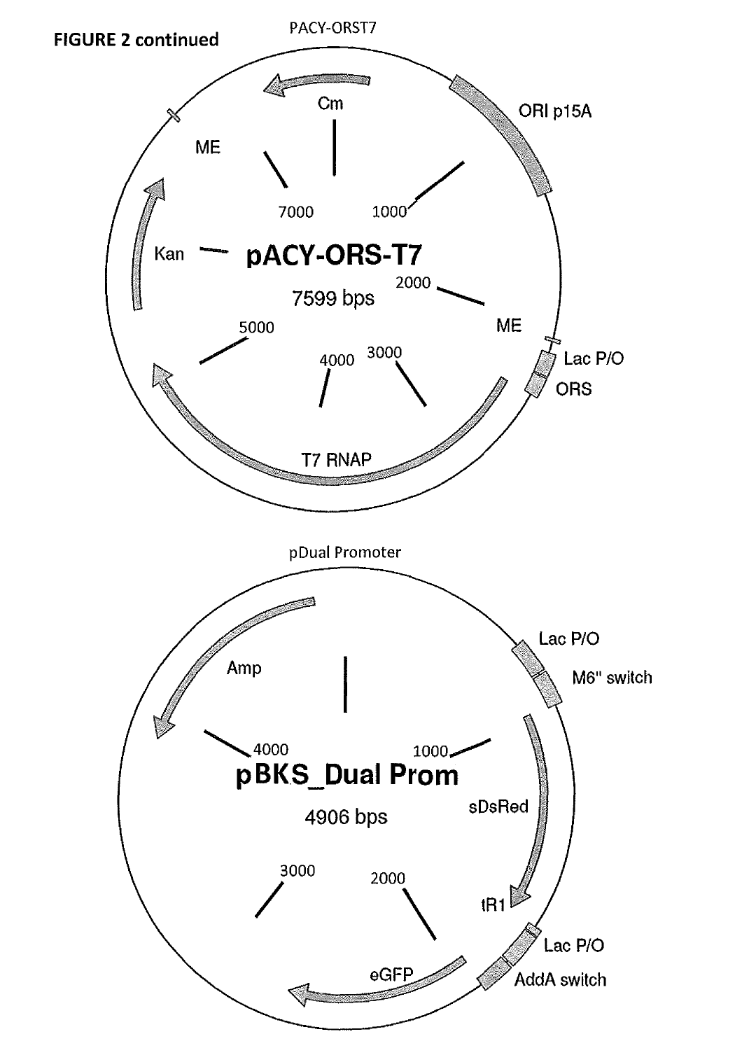

FIG. 5 shows the ligand effects on eGFP expression regulated by M6″ riboswitch;

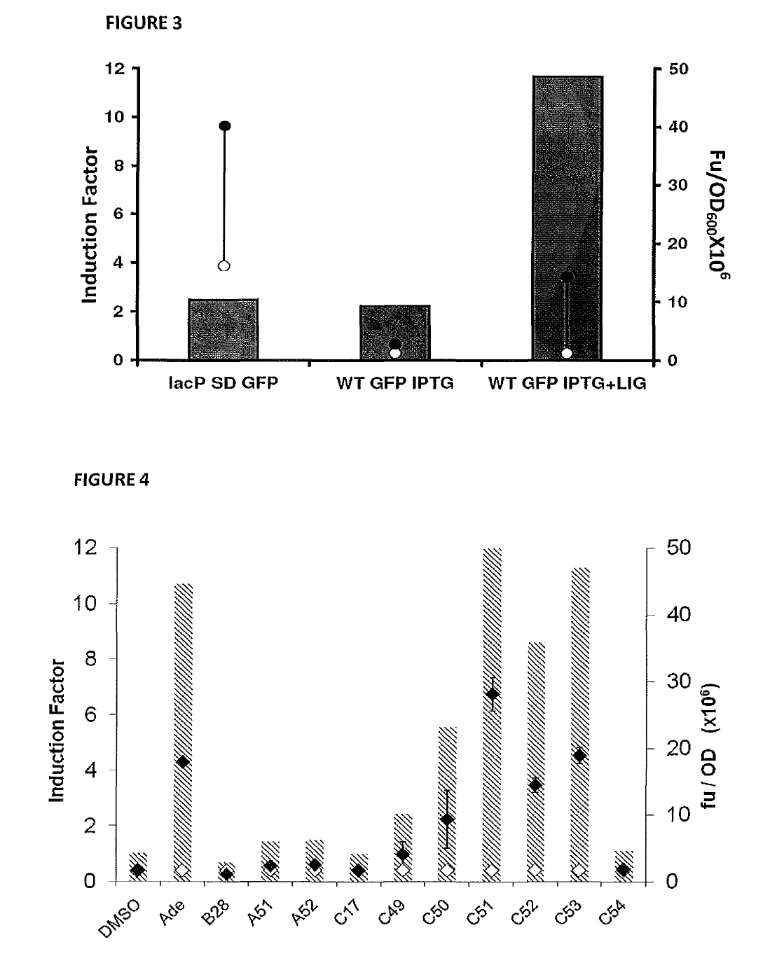

FIG. 6 shows the ligand effects on DsRED expression regulated by M6″ riboswitch;

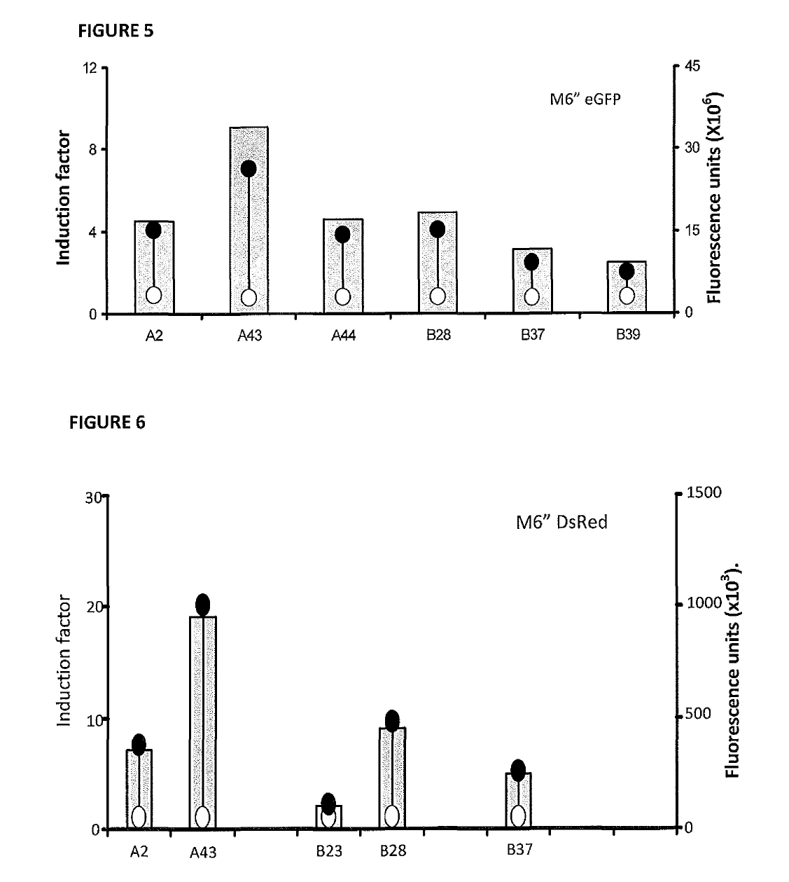

FIG. 7 shows the ligand effects on eGFP expression regulated by M6″ riboswitch;

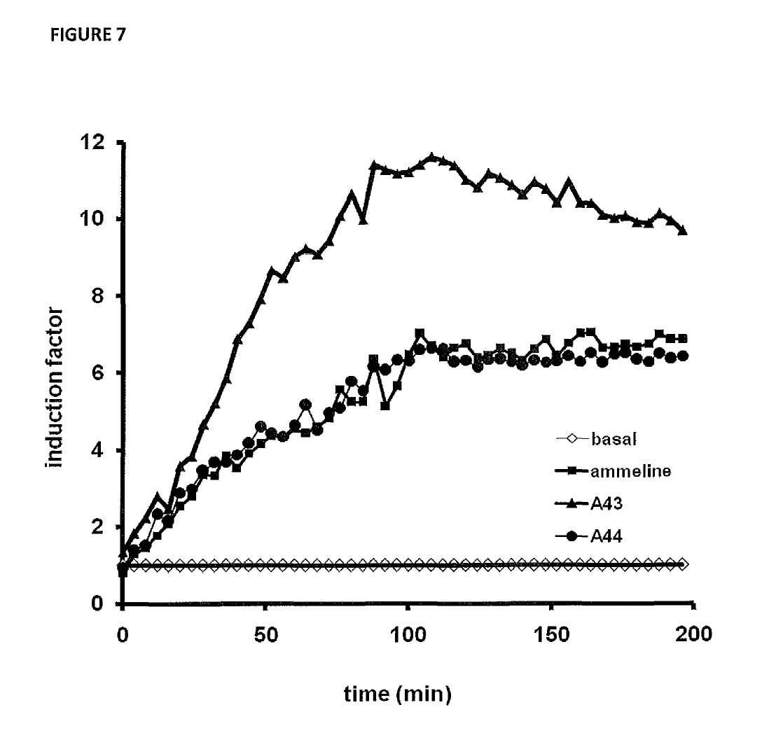

FIG. 8 shows the effect of a ligand and IPTG on eGFP expression regulated by an M6″ riboswitch;

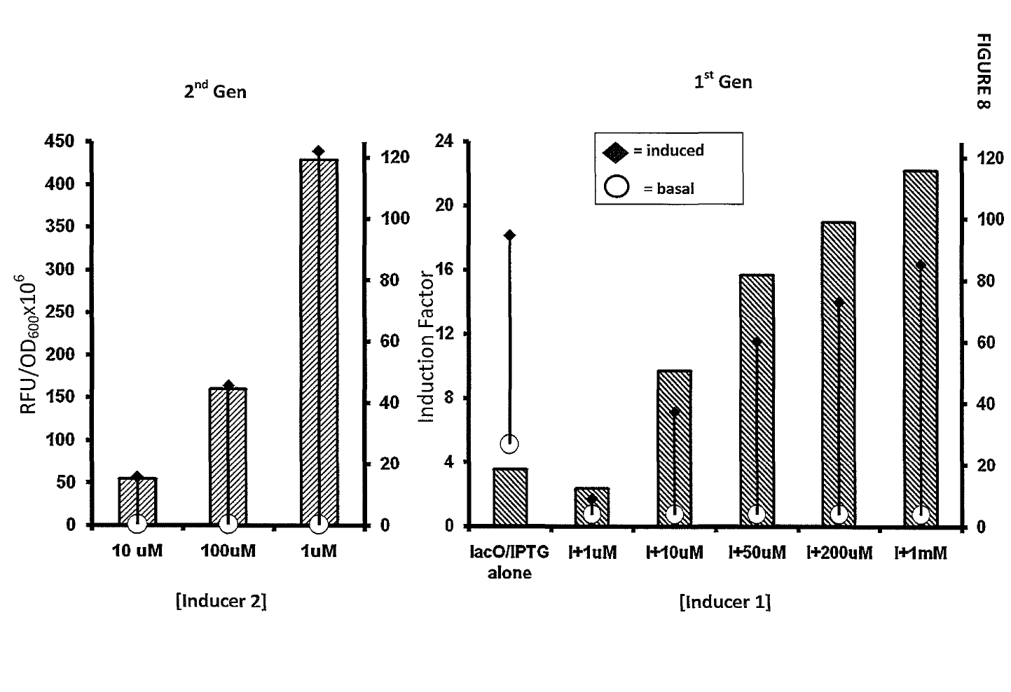

FIG. 9 shows the ligand and IPTG effects on eGFP expression regulated by M6C″ riboswitch;

FIG. 10 shows the effect of ligand effects on eGFP expression regulated by M6C″ riboswitch;

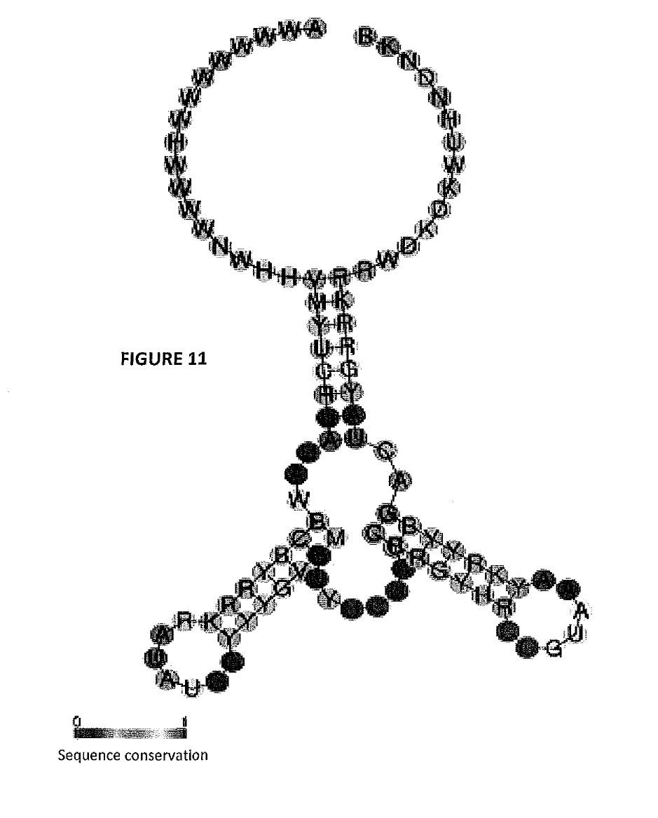

FIG. 11 shows the consensus purine riboswitch aptamer sequence. Rfam accession number RF00167

FIG. 12 shows the nucleotide sequences of purine aptamer sequences;

FIG. 13 shows the sequences of AddA derived riboswitches;

FIG. 14 shows the consensus of a PreQ riboswitch and structural configuration of a PreQ riboswitch having RFMA accession number RF00522;

FIG. 15 shows the upstream region of queC ORF. Pre Q1 riboswitch shown in bold

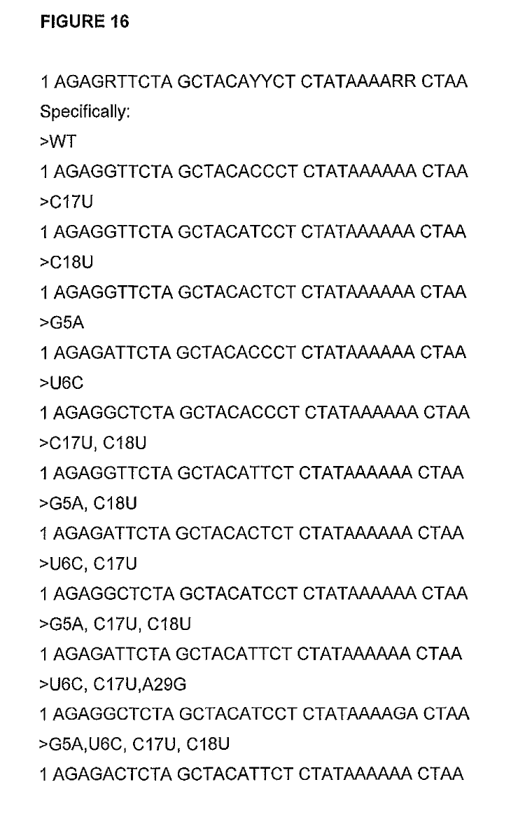

FIG. 16 shows a consensus mutant sequence of a PreQ riboswitch and specific mutants thereof. The present invention should be taken as encompassing any possible mutant sequence combinations from nucleotide substitutions at nucleotides R5, Y17, Y18, R29, R30, where R=purine (adenine or guanine) and Y=pyrimidine (cytosine and uracil (thymidine in DNA));

FIG. 17 shows an example of anti-sense RNA sequence. Antisense complimenting the drug target sequence (RodZ);

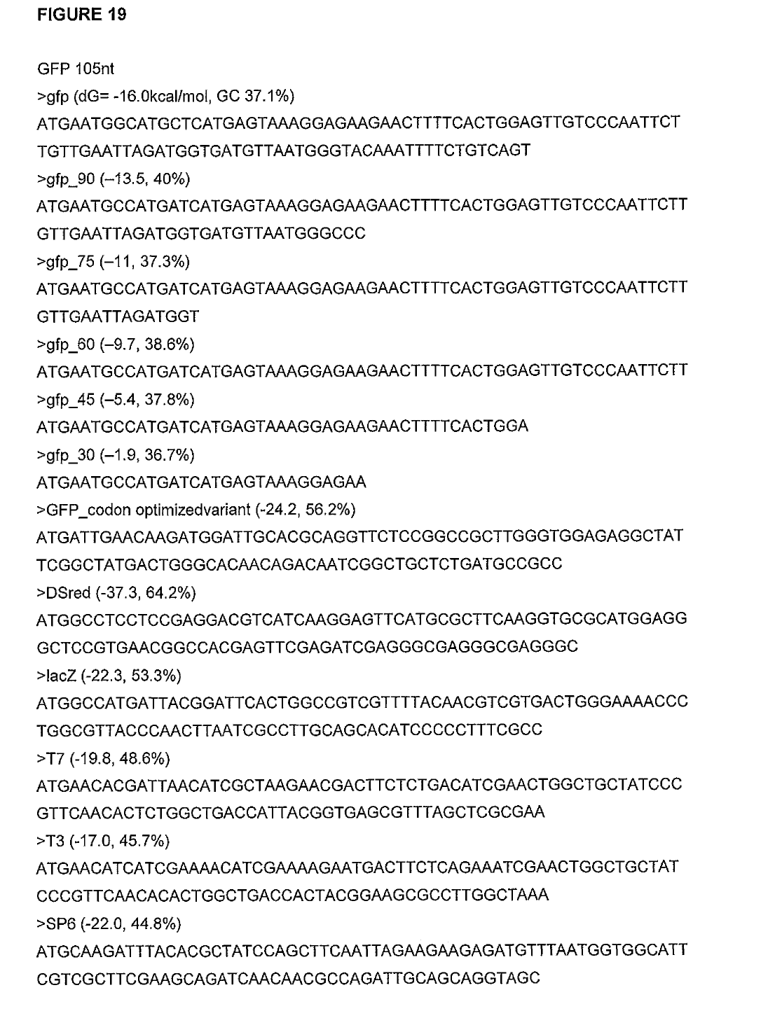

FIG. 18 shows an example of antibiotic drug target sequence controlled by PreQ riboswitch (PreQ1-MreB). PreQ1 riboswitch controlling the expression of the MreB gene, to create conditional mutant for drug candidate screening;

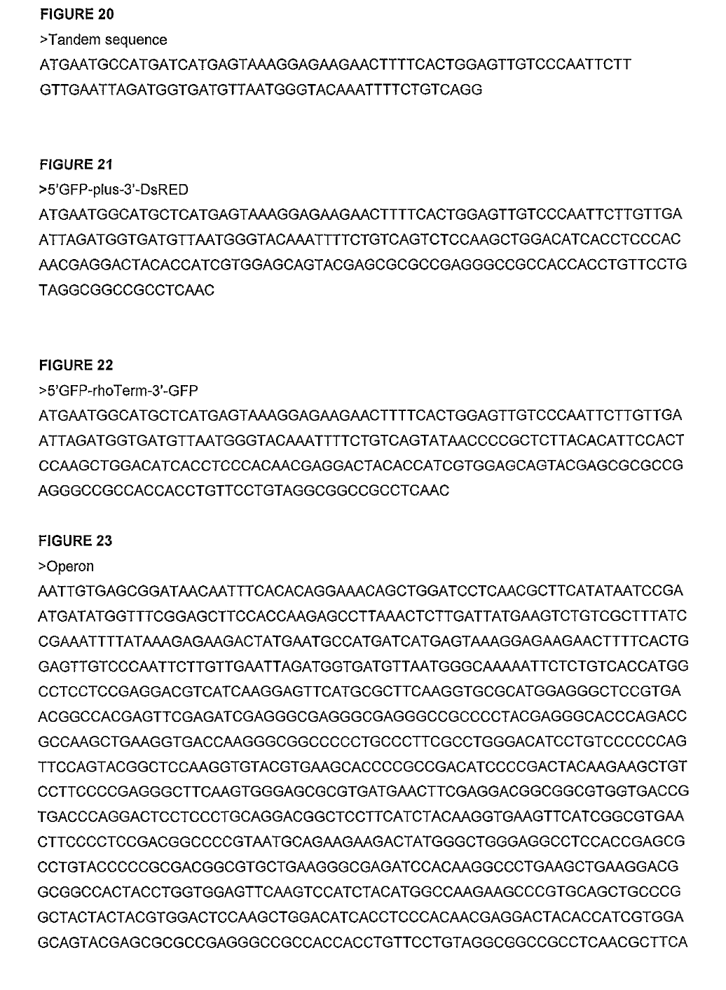

FIG. 19 shows the examples of possible spacer sequences for use in the present invention, also showing Gibbs free energy calculations produced using DINAmelt (Markham 2005) (dG), GC content shown.

FIG. 20 shows an example spacer used to separate tandem riboswitches;

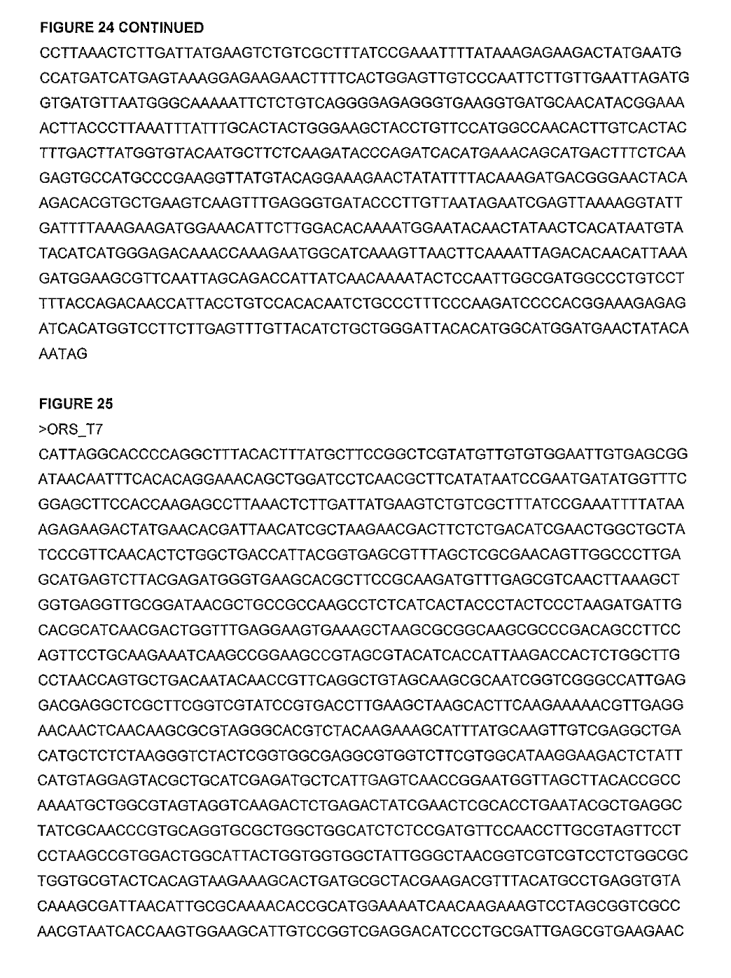

FIG. 21 shows an example spacer used to separate tandem riboswitches;

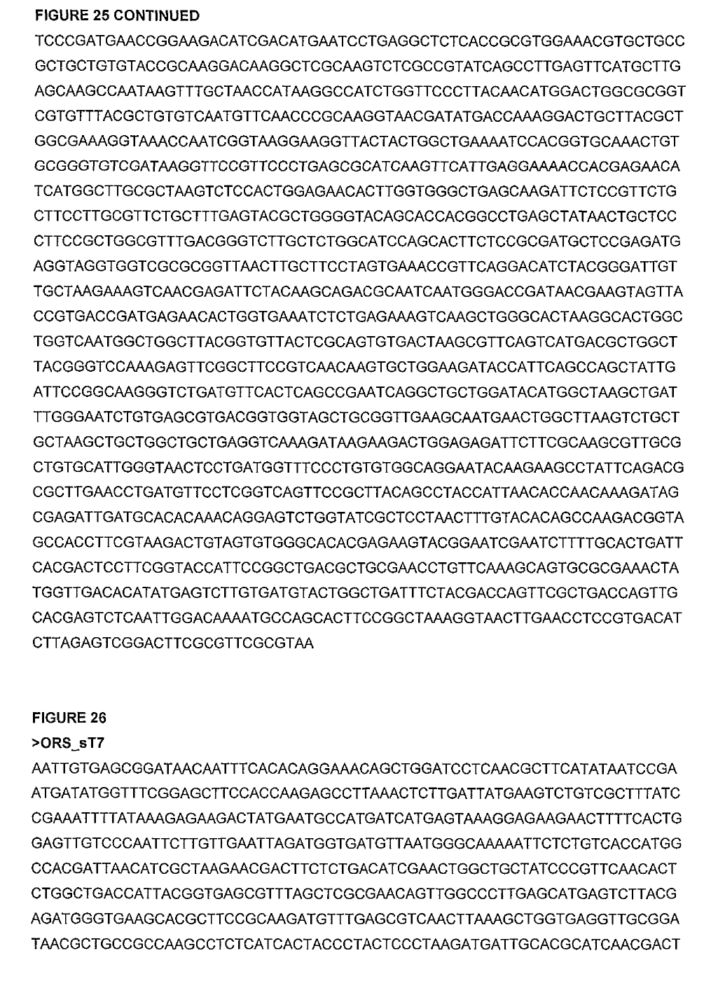

FIG. 22 shows an example spacer used to separate tandem riboswitches;

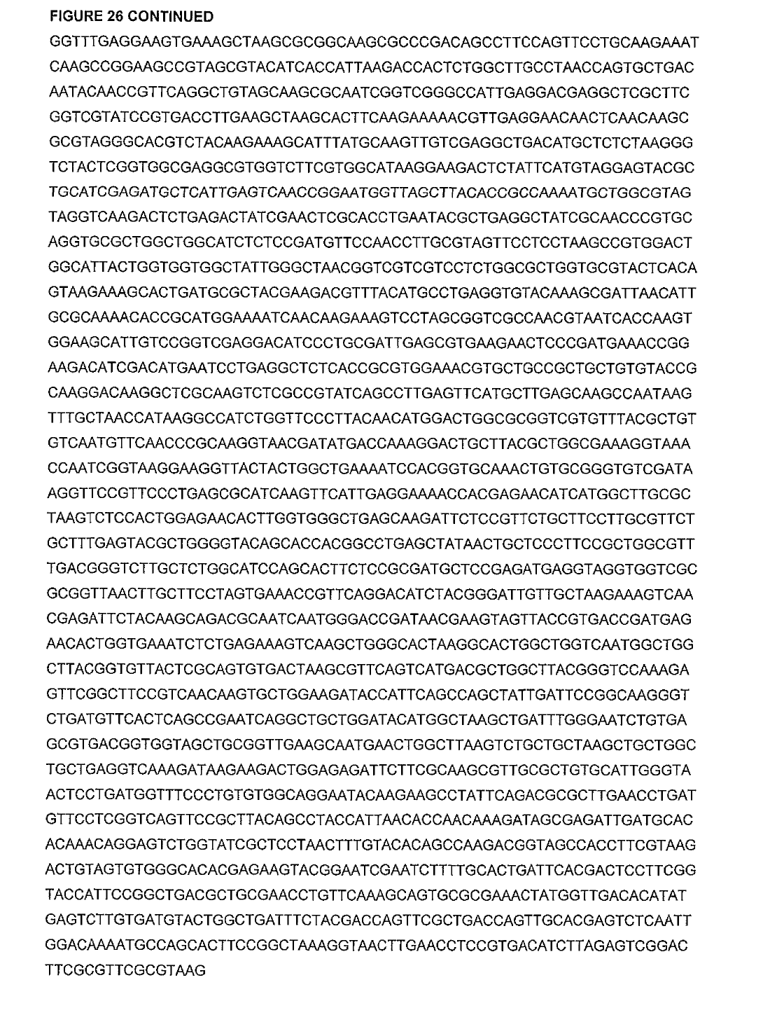

FIG. 23 shows an operon sequence, composed of lacUV5 promoter/operator, M6 riboswitch, DsRED gene, AddA riboswitch, eGFP gene;

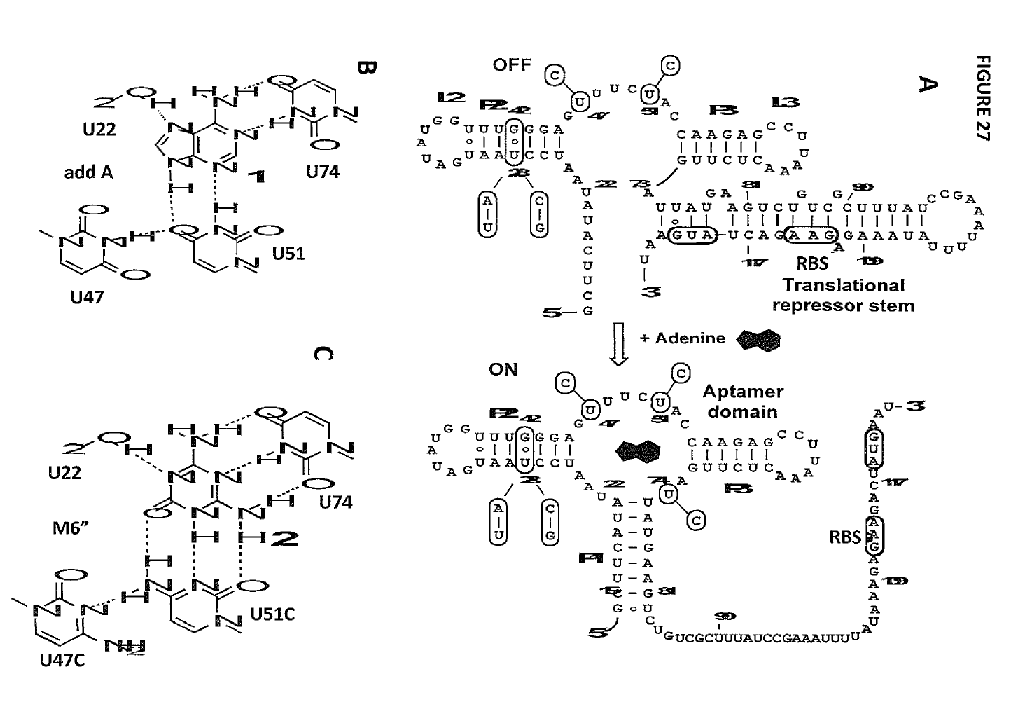

FIG. 24 shows a Dual Promoter sequence, composed of lacUV5 promoter/operator, M6 riboswitch, DsRED gene, lambda tR1 terminator, lacUV5 promoter/operator, AddA riboswitch, eGFP gene promoter;

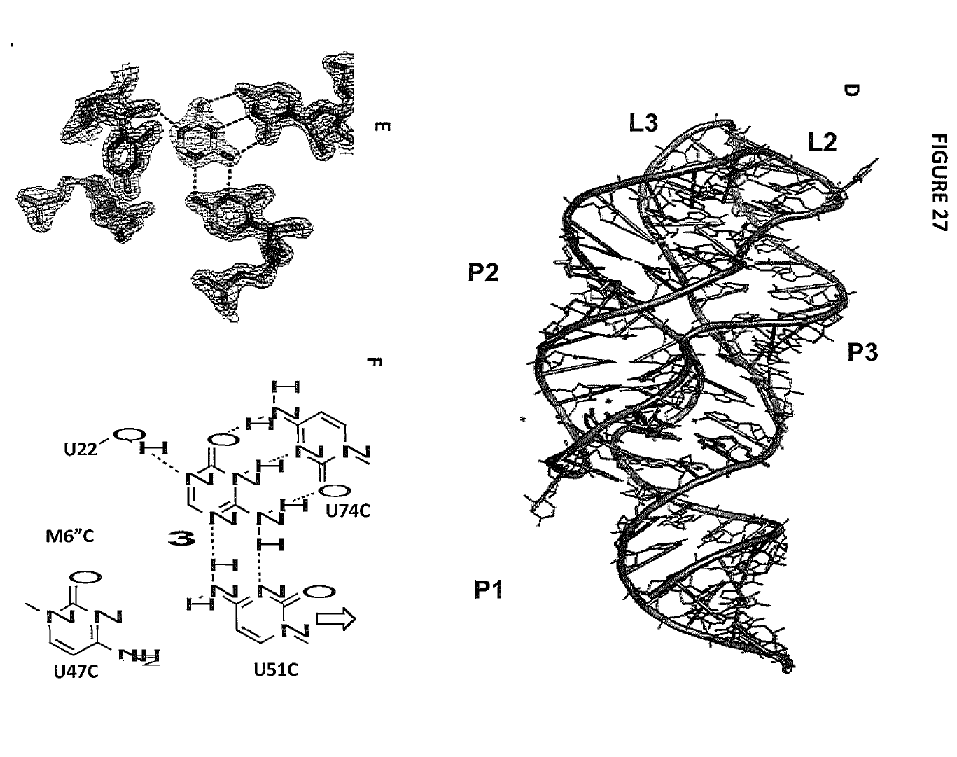

FIG. 25 shows an orthogonal Riboswitch T7 sequence, composed of lacUV5 promoter/operator, M6″ riboswitch, T7 RNA polymerase;

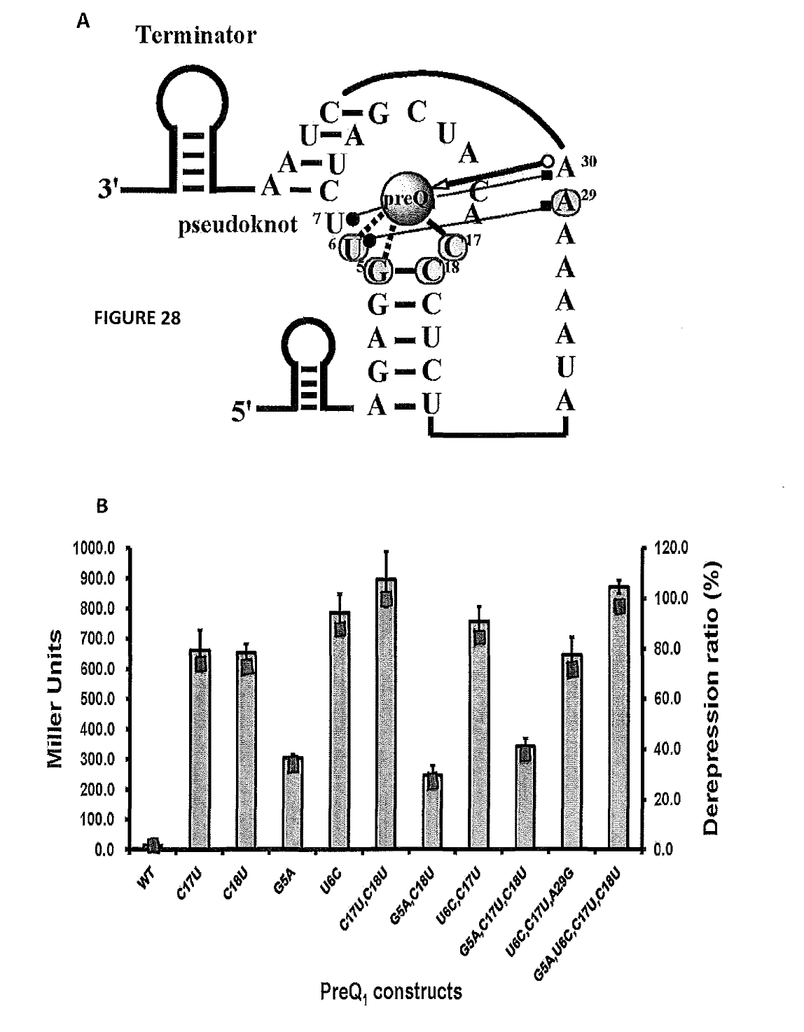

FIG. 26 shows orthogonal Riboswitch spacer T7 sequence, composed of lacUV5 promoter/operator, M6″ riboswitch, spacer sequence, T7 RNA polymerase;

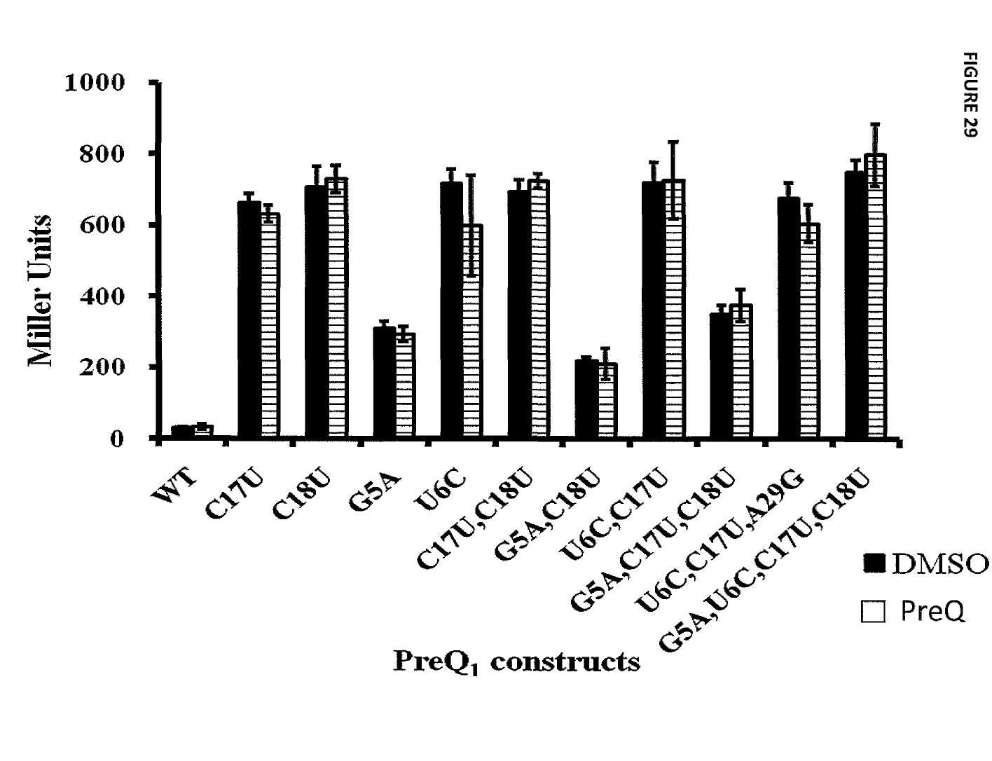

FIG. 27 shows (A) Secondary structure model of the parental add A-riboswitch. In the OFF-state the ribosome binding site (RBS) is sequestered in a repressor stem loop blocking translation. When present at sufficient concentrations adenine binds to the aptamer domain releasing the RBS. (B) add A-riboswitch aptamer domain showing H-bonding contacts to the ligand adenine 1. (C) Model of the mutant M6″ aptamer (U28G, G42C, U47C, U51C)H-bonding with ammeline 2. (D) 1.7 Å X-ray crystal structure of the mutant M6C″ (U28G, G42C, U47C, U51C, U74C) aptamer domain in complex with azacytosine 3. (E) Electron density contour map showing contacts between M6C″ and azacytosine 3, with H-bonding patterns shown below (F). Note the C51 translocation relative to the addA structure.

FIG. 28 shows the lacZ expression profiles of variant preQ₁ riboswitches. A Sequence of preQ₁ riboswitch constructs. The mutation sites are circled. The hydrogen bond interactions between preQ₁ binding residues and ligand preQ₁ are presented as Watson-Crick pairs ( [symbol] ), Cis Watson-Crick/Hoogsteen ( [symbol] ), Trans Watson-Crick/Sugar Edge ( [symbol] ) and single hydrogen bond interaction (...). B The regulation of β-galactosidase activity by the wild-type preQ₁ riboswitch and by the preQ₁ riboswitch mutants. The β-galactosidase activities are presented as Miller Units (left axis indicated by the blue bars) and the error bars represent the mean±SD of four replicates. The derepression ratios (right axis indicated as brown squares) are calculated by comparing with the C17U, C18U mutant strain;

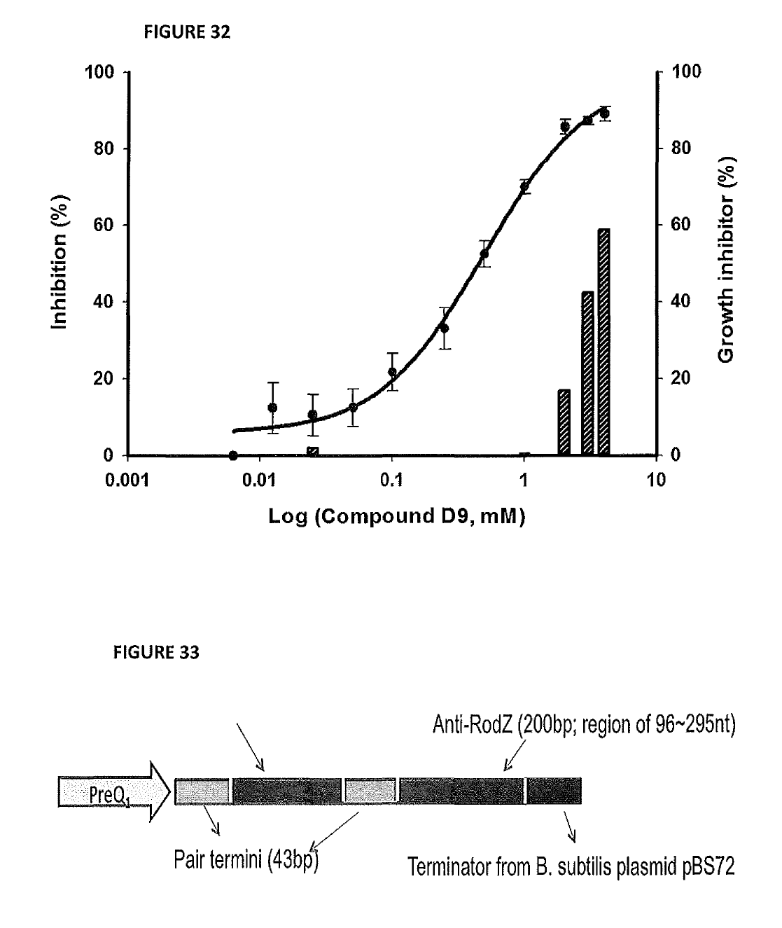

FIG. 29 shows the effect of the PreQ₀ ligand on the lacZ expression of variant preQ₁ riboswitches at 16° C.;

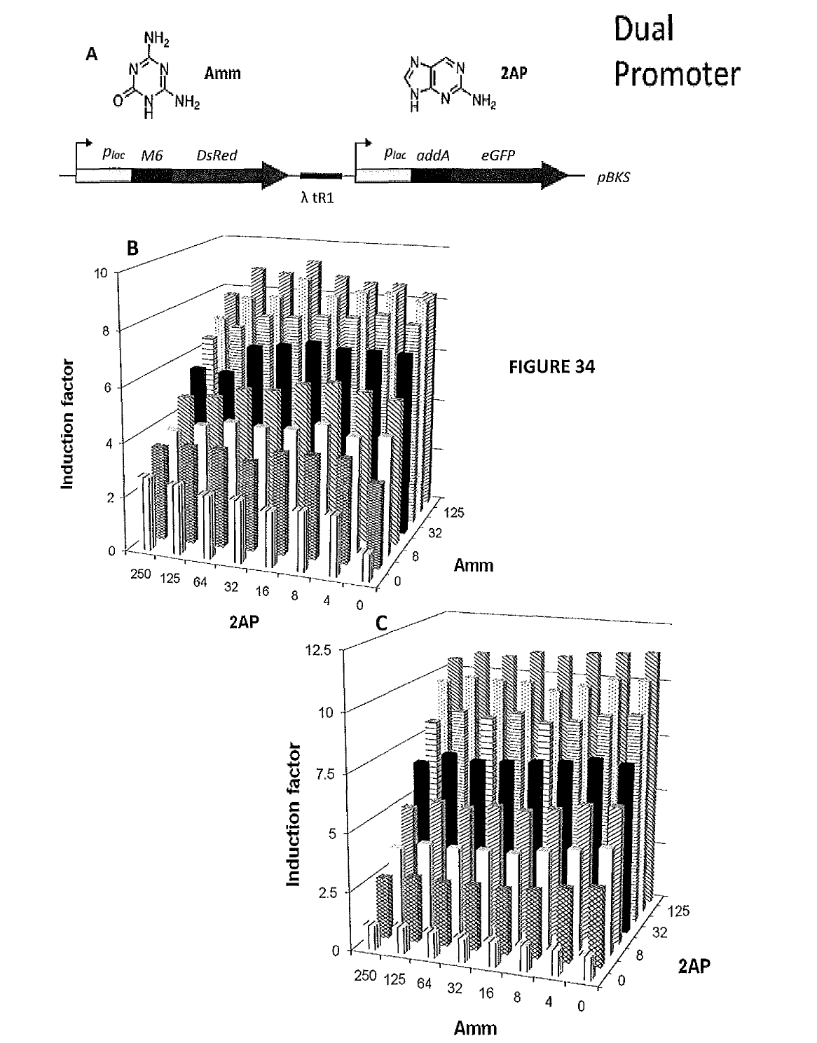

FIG. 30 shows the effect of the ligand D6 on the lacZ expression of variant preQ₁ riboswitches;

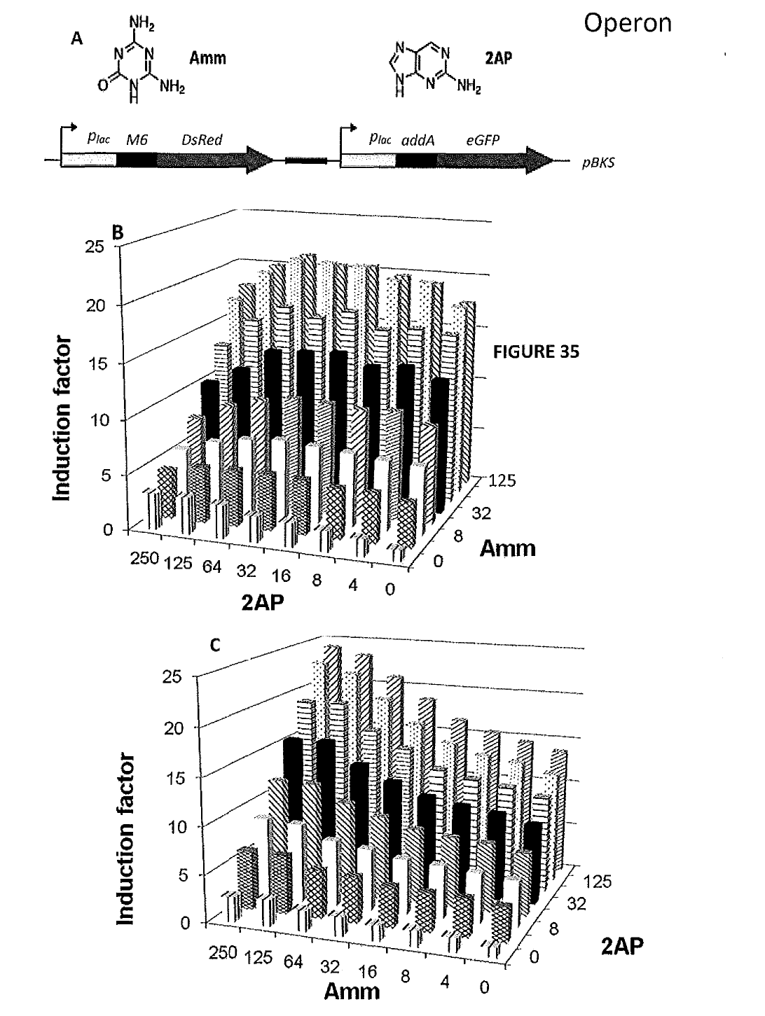

FIG. 31 shows the effect of the ligand D9 on the lacZ expression of variant preQ₁ riboswitches;

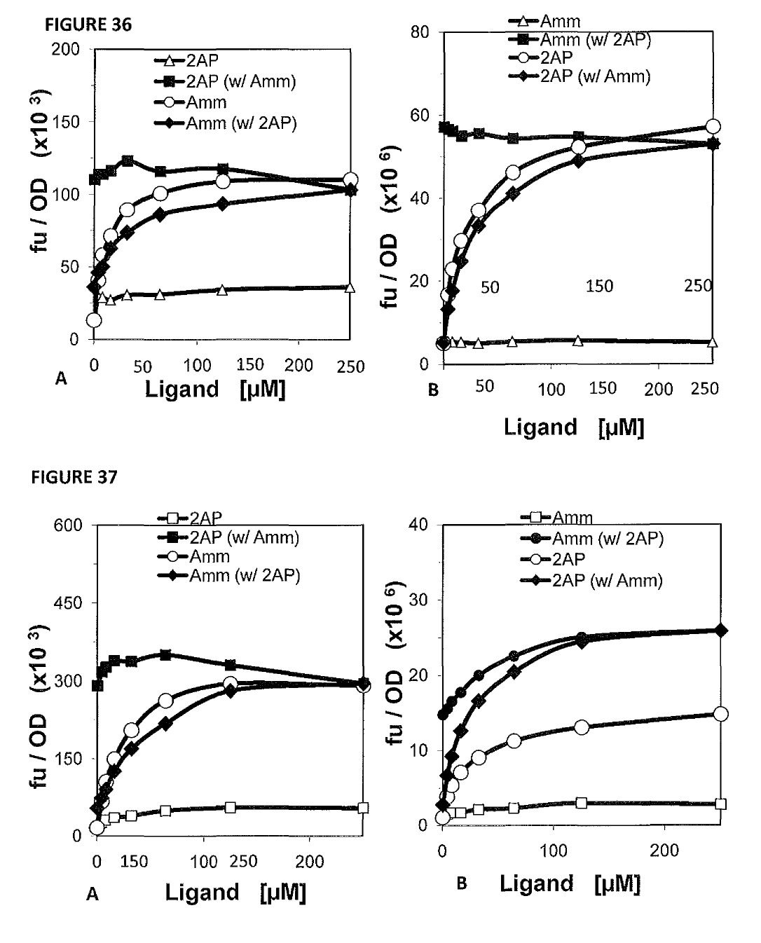

FIG. 32 shows the dose-response curves and growth inhibition of the compound D9. The dose-response curve with IC₅₀ values 0.498 mM was obtained by the in-vivo lacZ activity assay with an initial concentration of 6.25 μM to a final concentration of 4 mM. The IC₅₀ value was calculated by non-linear regression analysis (four parameter logistic equation) using Sigmaplot 12.0;

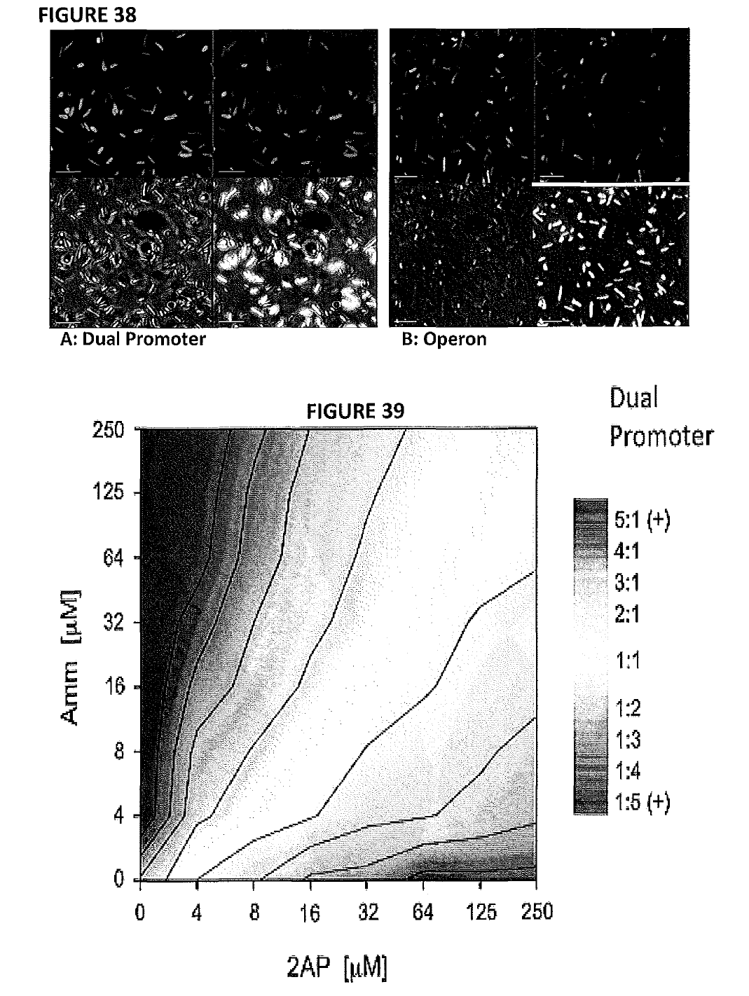

FIG. 33 shows PreQ Applications with Target genes rodZ and mreB;

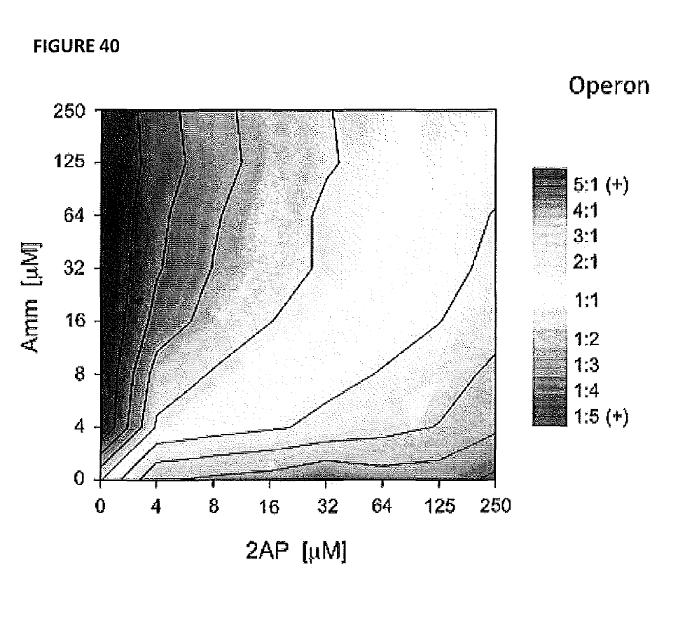

FIG. 34 shows multicomponent riboswitch gene expression systems. A Selective inducer molecules ammeline, and 2-aminiopurine. Dual Promoter construct comprised of promoter/operator (lacP/O), the M6 ammeline responsive riboswitch in front of the red fluorescent protein DsRed, lambda tR1 terminator, a second lacP/0 lac promoter, the add-A 2-aminopurine responsive riboswitch in front of the green fluorescent protein eGFP. B DsRed and C eGFP Multi-variant dose matrix analysis of induction factors;

FIG. 35 shows multicomponent riboswitch gene expression systems. A Selective inducer molecules ammeline, and 2-aminopurine. Operon construct comprised of promoter/operator (lacP/O), the M6″ ammeline responsive riboswitch in front of the red fluorescent protein DsRed, the add-A 2-aminopurine responsive riboswitch in front of the green fluorescent protein eGFP. B DsRed and C eGFP multi-variant dose matrix analysis of induction factors;

FIG. 36 shows dose-response curves for dual promoter construct: (A) DsRed; (B) eGFP expression;

FIG. 37 shows dose-response curves for operon construct: (A) DsRed; (B) eGFP expression;

FIG. 38 shows the results of fluorescent microscopy: A Dual promoter construct B Operon construct. Induced with IPTG (1 mM), ammeline 1 (250 μM), and 2-aminopurine 2 (250 μM). (top left) GFP channel, (top right) DsRED channel, (bottom left) Phase contrast (bottom right) Overlay of channels. Scale bar equal to 5 μm for reference.

FIG. 39 shows a multi-variant dose matrix analysis of absolute normalized protein expression levels showing dual promoter relative protein expression stoichiometry ratio. Gradient lines range from 1:5 on the left hand side of the matrix, to 4:1 on the lower right hand side.

FIG. 40 shows a multi-variant dose matrix analysis of absolute normalized protein expression levels showing operon relative protein expression stoichiometry ratio. Gradient lines range from 1:5 on the left hand side of the matrix, to 4:1 on the lower right hand side.

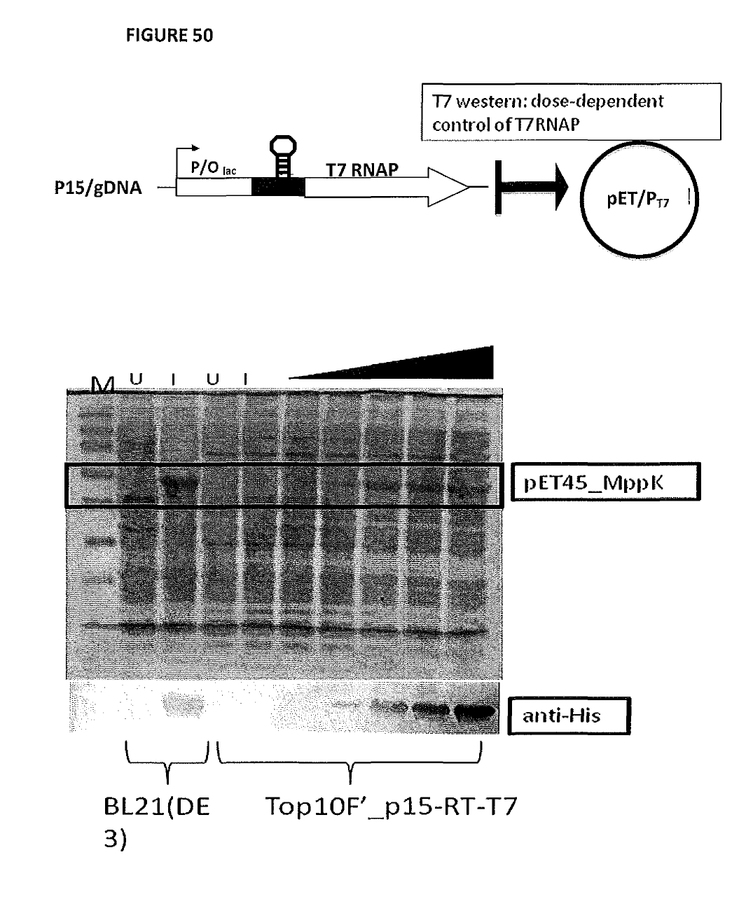

FIG. 41 shows genetic constructs containing M6″ riboswitch, T7 RNAP, second construct containing T7 promoter ahead of any gene of interest. P_{lac}=lac UV5 promoter, O_{lac}=lac operator, P_{T7}=T7 promoter, P_{T3}=T3 promoter, MCS=Multi-cloning site, EP=expression plasmid e.g. pET, POI=protein of interest

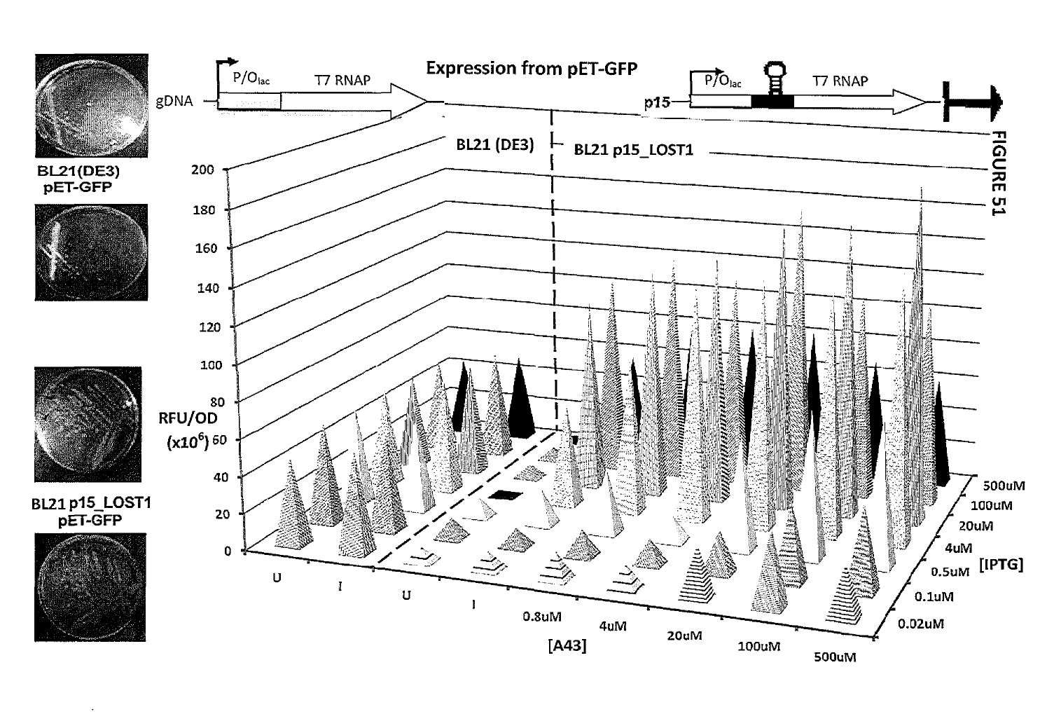

FIG. 42 shows multi component genetic containing dicistronic M6″ riboswitch-T7 RNAP, addA riboswitch-T3 RNAP and a second construct containing the T7 and T3 promoters sequence ahead of any genes of interest, (referred to as pTTv7). P_{lac}=lac UV5 promoter, O_{lac}=lac operator, P_{T7}=T7 promoter, P_{T3}=T3 promoter, MCS=Multi-cloning site, EP=expression plasmid e.g. pET, mPOI=multimeric protein of interest

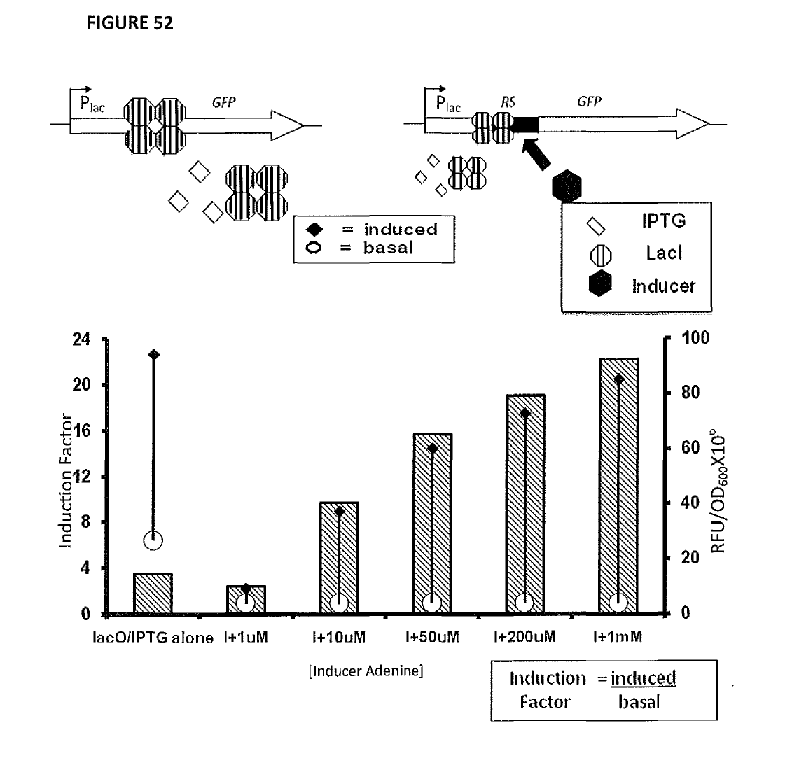

FIG. 43 shows differences in lacZ induction by riboswitches (both addA and M6) in genetic constructs in which a spacer is absent, or in which a spacer is present (constituting examples of genetic constructs in accordance with the first aspect of the invention).

FIG. 44 shows differences in induction of the fluorescent marker DsRed by riboswitches in genetic constructs in which a spacer is absent, or in which a spacer is present (constituting examples of genetic constructs in accordance with the first aspect of the invention).

FIG. 45 shows differences in induction of T7 RNA polymerase by riboswitches in genetic constructs in which a spacer is absent, or in which a spacer is present (constituting examples of genetic constructs in accordance with the first aspect of the invention).

FIG. 46 shows differences in T7 RNA polymerase expression in response to increasing amounts of a riboswitch ligand for comparator genetic constructs in which a spacer is absent, or for genetic constructs of the invention incorporating spacer sequences of variable lengths (30, 45, 60, 75, 90, 93, 96, 99, 102, 105, 120, 135, 150 or 165 nucleotide lengths) and for a control genetic construct lacking a spacer sequence (designated “nts”). Expression is induced using IPTG (0.5 mM) and increasing concentrations of A43 (respectively 0, 5, 10, 25, 50, 100, 250 uM) shown in the third to eighth columns.

FIG. 47 shows three graphs representing differing induction of T7 RNA polymerase expression in response to increasing amounts of a riboswitch ligand for genetic constructs of the invention incorporating spacer sequences of variable lengths (as before) and for a control genetic construct lacking a spacer sequence.

FIG. 48 compares T7 RNA polymerase expression in genetic constructs of the invention in which lac I is inserted in either the same or the opposite direction to the T7 RNA polymerase open reading frame.

FIG. 49 demonstrates the decoupled concept where an inducible promoter coupled to a riboswitch permits dose dependent control over the expression pf T7 RNAP, and by using a 2^{nd} generation construct with the T7 promoter sequence and a gene of interest cloned downstream from said promoter, dose dependent control of gene (of interest) expression is permitted in a de-couplled manner.

FIG. 50 shows a systems containing two constructs, first a construct that permits control of the T7 RNAP (either BL21(DE3), or the low copy vector p15LOST (containing the lac repressor, the orthogonal ricoswicth (M6″), a spacer sequence and the T7RNAP). The second construct a pET vector containing the 50 kDa protein MppK. Here the de-couple riboswitch construct is shown to permit dose-dependent regulation of the gene of interest (MppK, upon addition of IPTG and riboswitch inducer A43).

FIG. 51 shows a systems containing two constructs, first a construct that permits control of the T7 RNAP either BL21(DE3), or an E. coli strain containing the low copy vector p15LOST (containing the lacI repressor, the orthogonal ricoswicth (M6″), a spacer sequence and the T7RNAP). The second construct a pET vector containing the GFP protein Here the de-coupled riboswitch construct is shown to permit dose-dependent regulation of the gene of interest (GFP), upon addition of IPTG and riboswitch inducer A43).

FIG. 52 shows GFP expression data for constructs with and without the riboswitch (AddA), with addition fo IPTG and riboswitch inducer (A1).

FIG. 53 shows GFP expression data for constructs with and without the riboswitch (M6″), with addition fo IPTG and riboswitch inducer (A43).

FIG. 54. shows the cellular distribution of GFP expression analysed by FACS.

FIG. 55 shows the sensitivity of the B. subtilis MreB knockout, and the reconstituted MreB under the control of the repressible PreQ C17U riboswitch mutant, providing a conditional mutant upon addition of D9

FIG. 56 shows in a first panel the genetic construct pMAST3-h6, transcriptionally regulated by a lac promoter/operator containing the addA riboswitch—A1 ligand combination, controlling the expression if the T3 RNA polymerase-h6; in a second panel, SDS analysis of cell lysates expression induced with 0.5 mM IPTG, and A1 at 0.8, 4, 20, 100, 250, 250 uM is shown, illustrating dose-dependent control of expression; the third panel shows Western blot analysis with anti-his6 antibody. The construct is omposed of lacUV5 promoter/operator, AddA riboswitch, spacer, T3 RNA polymerase with a C-his6 tag.

FIG. 57 shows, in panel “a”, a dual promoter construct transcriptionally regulated by a lac promoter/operator, where the M6″ riboswitch—A43 ligand combination control the expression of the spT7 RNA polymerase, separated by at tR1 transcriptional terminator, a second lac promoter/operator and the addA riboswitch—A1 ligand combination control the expression if the T3 RNA polymerase (pTTv9)(top); and a dicistronic operon transcriptionally regulated by the lac promoter/operator, where the M6″ riboswitch—A43 ligand combination control the expression of the spT7 RNA polymerase, and the addA riboswitch—A1 ligand combination control the expression if the spT3 RNA polymerase (pTTv7) (below). Panel “b” shows SDS analysis of cell lysates; panel “c” shows Western blot analysis with anti-T7RNAP antibody; and panel “d” shows Western blot analysis with anti-his6 antibody

FIG. 58 shows the dose dependent control of eGFP expression from constructs WT (AddA) and M6″(ORS), with selective inducers Adenine (Ad) Ammeline (Am), pyrimido[4,5-d]pyrimidine-2,4-diamine (A43)

FIG. 59 shows isothermal titration calorimetry plots between the riboswitch aptamers WT (AddA) and M6″(ORS), pyrimido[4,5-d]pyrimidine-2,4-diamine (A43)

FIG. 60, shows isothermal titration calorimetry plots between the riboswitch aptamers WT (AddA) and M6″(ORS), 2,4-Diaminopteridine (A44)

FIG. 61 Isothermal titration calorimetry plots between the riboswitch aptamers M6C″ and ligands

FIG. 62 shows the structure and sequence of the AddA, M6″ and M6C″ aptamers FIG. 63 shows additional examples of spacer sequences further to (FIG. 19) Gibbs free energy calculated using DINAmelt (Markham 2005) (dG), GC content shown.

FIG. 64 shows pMAST-h6 sequence, composed of lacUV5 promoter/operator, AddA riboswitch, spacer, T3 RNA polymerase with a C-his6 tag

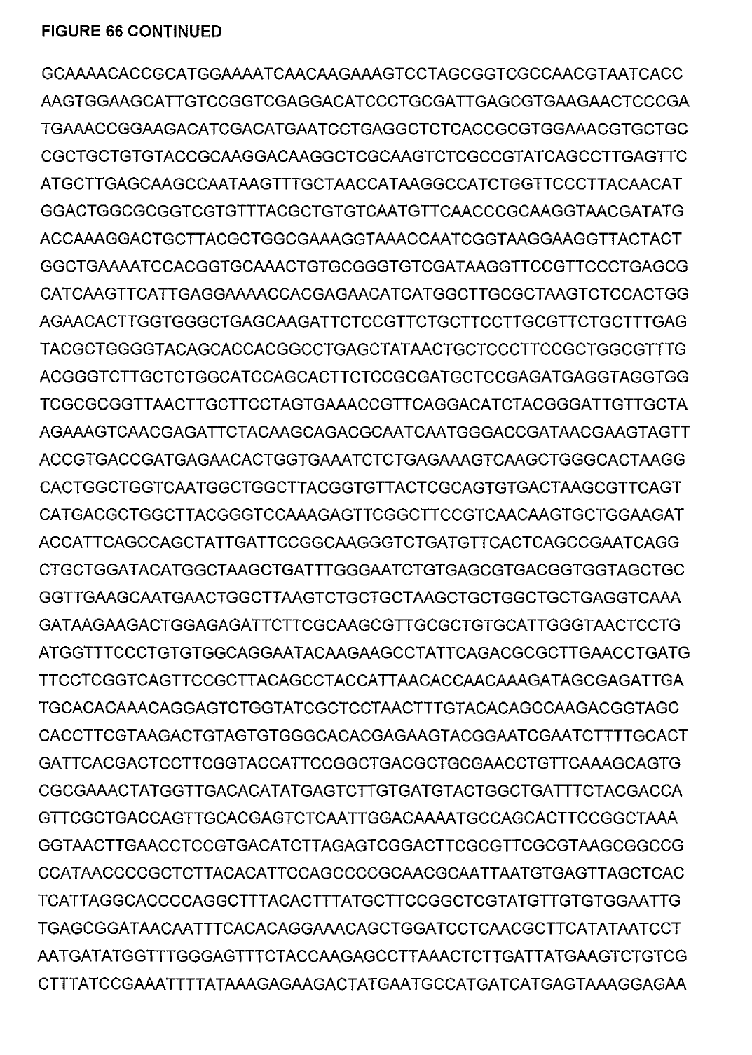

FIG. 65 shows the pTTv7 (operon) sequence, composed of lacUV5 promoter/operator, M6″ riboswitch, spacer, T7 RNAP gene, AddA riboswitch, spacer T3 RNAP gene.

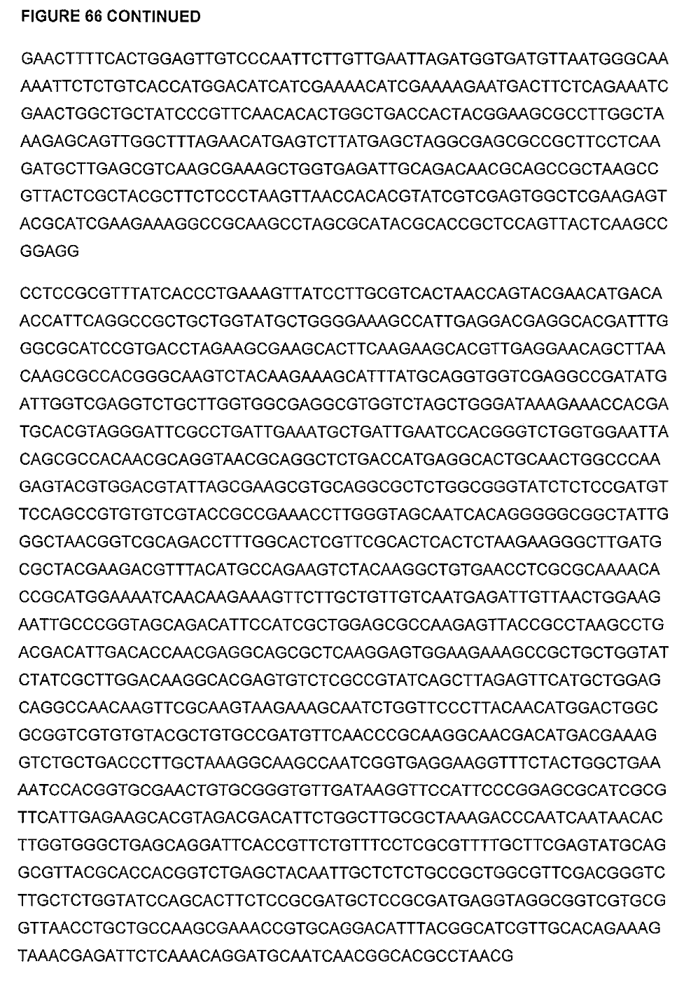

FIG. 66 pTTv9 (dual promoter) sequence, composed of lacUV5 promoter/operator, M6″ riboswitch, spacer, T7 RNAP gene, lambda tR1 terminator, lacUV5 promoter/operator, AddA riboswitch, spacer T3 RNAP gene.

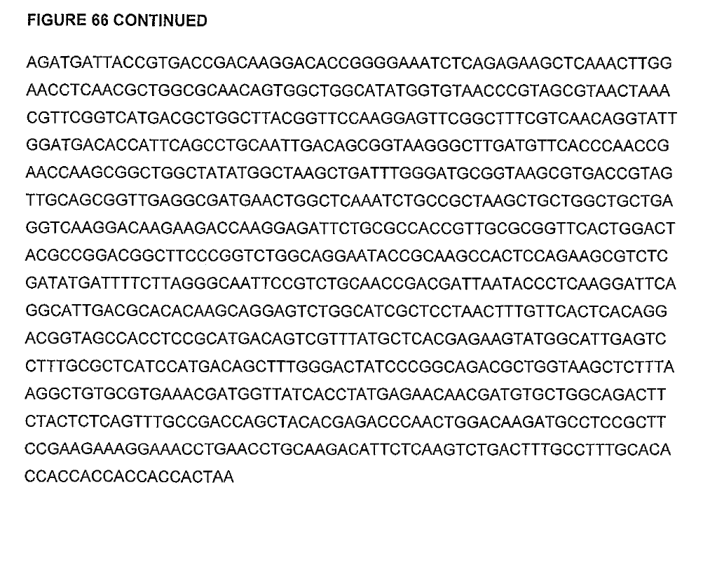

FIG. 67 shows the dose-response curves for the dual promoter construct (a) DsRed (b) eGFP expression (update of FIG. 36), and for the operon construct (c) DsRed (d) eGFP expression (update of FIG. 37).

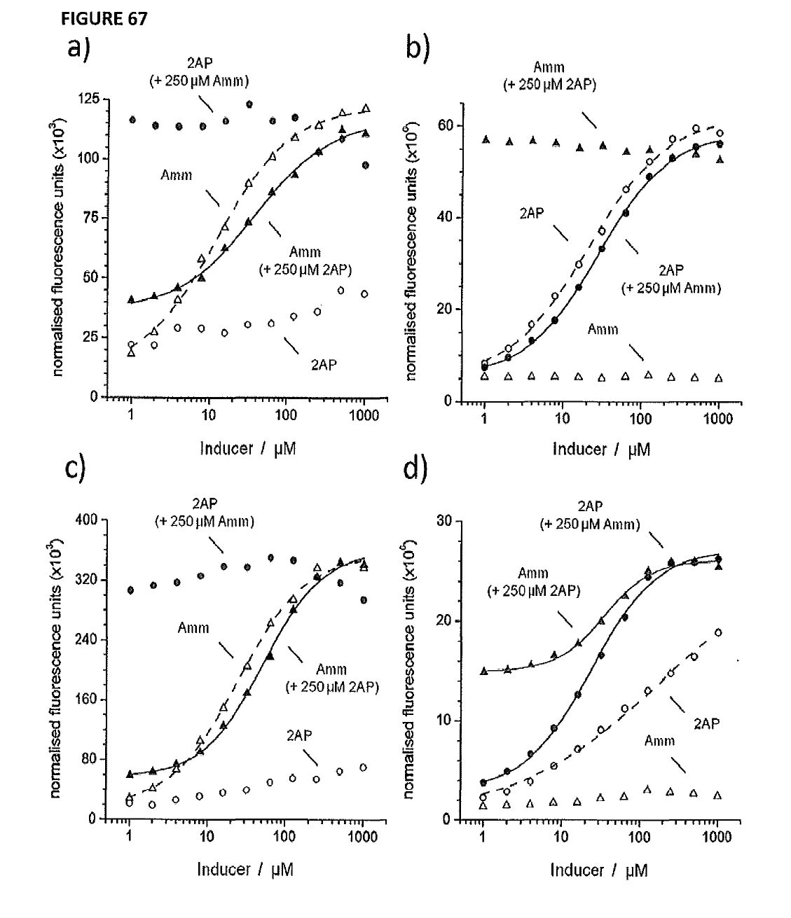

### DETAILED DESCRIPTION

The present invention provides novel engineered expression systems comprising riboswitches, which allow orthogonally selective, tunable and/or dose dependent control of gene expression in response to a ligand. The different aspects of the present invention have applicability in a range of different fields, including for example functional genomics, target validation and drug development, systems chemical biology, NOR boolean logic gates, synthetic biology, plant biology, gene therapy, recombinant protein production, pathway engineering and directed evolution, bioremediation and metabolic engineering.

### DEFINITIONS

“Antisense sequence” nucleic acid molecules that are specifically hybridizable or specifically complementary to either RNA or the plus strand of DNA. In a cell, the antisense nucleic acids hybridize to the corresponding mRNA, forming a double-stranded molecule. The antisense nucleic acids interfere with the translation of the mRNA.

“Aptamer domain” is a part of a riboswitch which is capable of binding a ligand. Preferably, ligand binding results in a structural change in the expression platform of the riboswitch, which is operably linked to the aptamer domain, and effects regulation of the expression of the nucleic acid sequence which is regulated by the riboswitch. An aptamer domain is a nucleic acid sequence, preferably an RNA sequence, and preferably encoded by a DNA sequence. Binding of the aptamer domain to a ligand is preferably selective, to a particular class of type of ligands.

“Contiguous” such that the last nucleic acid residue of one nucleic acid molecule is adjacent to the first nucleic acid residue of another of the first, second or third nucleic acid molecules.

“Dynamic range” is defined as the difference between high and low protein levels.

“Expression” herein means either transcription or translation or both, i.e. From DNA to RNA, and/or RNA to protein or peptide. A riboswitch used in the present invention may operate at either the transcriptional or translational level.

“Expression platform” Within the context of the present invention, the portion of the riboswitch which mediates the effect on the nucleic acid expression is known as the expression platform. Preferably, the expression platform is operably linked to the aptamer domain of the riboswitch, preferably structurally linked, most preferably by a nucleic acid linker. Most preferred is that the aptamer domain is linked to the expression platform by a nucleic acid sequence. Preferably, the stem configuration of the portion of the expression domain changes configuration upon binding of a ligand molecule, such that a change in the configuration of the stem structure results in a corresponding change in the structure of the expression platform, between a first configuration which enhances expression of the nucleic acid sequence and a second configuration which inhibits expression of the nucleic acid sequence.

“Genetic construct” can include nucleic acid sequences that permit it to replicate in the host cell, such as an origin of replication. Examples include, but are not limited to a plasmid, cosmid, bacteriophage, or virus that carries exogenous DNA into a cell. A genetic construct can also include one or more cytotoxic genes, antisense molecules, and/or selectable marker genes and other genetic elements known in the art. A genetic construct can preferably transduce, transform or infect a cell, thereby causing the cell to express the nucleic acids and/or proteins encoded by the vector. A genetic construct optionally includes materials to aid in achieving entry of the nucleic acid into the cell, such as a liposome, protein coating or the like.

“Isolated” biological component (such as a nucleic acid or protein) has been substantially separated, produced apart from, or purified away from other biological components in the cell of the organism in which the component naturally occurs, i.e., other chromosomal and extra chromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

“Operably linked” a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame. Nucleic acid: a deoxyribonucleotide or ribonucleotide polymer in either single or double stranded form, and unless otherwise limited, encompasses known analogues of natural nucleotides that hybridize to nucleic acids in a manner similar to naturally occurring nucleotides.

“Orthogonal” in relation to the riboswitches disclosed herein means that they have greater selectivity for non-native, non-natural and/or synthetic ligands than for a natural, native ligand. More preferably, an orthogonal riboswitch responds to a non-native, non-natural and/or synthetic ligand to the same extent in terms of the effect on coding sequence expression as to a native, natural or intracellular ligand and shows substantially no response to a native, natural and/or intracellular ligand.

“Oligonucleotide”: a linear polynucleotide (such as DNA or RNA) sequence of at least 9 to 35 nucleotides, for example at least 15, 18, 24, 25, 27, 30, 50, 100 or even 200 nucleotides.

“Peptide”: a chain of amino acids of which is at least 4 amino acids in length. In one example, a peptide is from about 4 to about 30 amino acids in length, for example about 8 to about 25 amino acids in length, such as from about 9 to about 15 amino acids in length, for example about 9-10 amino acids in length.

“Promoter”: an array of nucleic acid control sequences that directs transcription of a nucleic acid. A promoter includes necessary nucleic acid sequences near the start site of transcription, such as, in the case of a polymerase ii type promoter, a TATA element. A promoter also optionally includes distal enhancer or repressor elements that can be located as much as several thousand base pairs from the start site of transcription. Both constitutive and inducible promoters are included.

“Riboswitch” means an expression control element, which is preferably in the form of an RNA molecule. A riboswitch comprises a ligand binding domain, also referred to herein and in the art as an aptamer domain, and a regulatory domain which influences gene expression, and is also known as the expression platform of the riboswitch. For a riboswitch to respond to the effects of a particular ligand by regulating gene expression, it must comprise a ligand binding domain and a regulatory domain.

“Regulatory sequence” is a sequence which serves to control an element of transcription or translation of a coding sequence. For example, it may encode an antisense RNA sequence which regulates expression of a coding sequence by binding thereto or to a transcript thereof. Alternatively, it may encode a regulatory element such as a promoter, enhancer or transcription factor binding site, or may encode a gene product which controls expression, such as a transcription factor, a polymerase, a repressor, a translation initiation factor.

“Secondary structure” in a nucleic acid sequence is the three dimensional structure formed by hydrogen bonds between the two bases. Secondary structure includes Watson and Crick base pairing, wobble pairings, stems and loops, hairpins, pseudoknots, duplexes, triplexes, tetraplexes. When measured using a predictive tool for secondary structure such as Genscan (Burge et al; J. Mol. Biol. 268, 78-94); Genviewer. Suitable folding packages for determining secondary structure include RNA Vienna (Hofacker NAR 1: 3429 (2003)), mfold (Zuker NAR 31: 3406 (2003), DINAMelt (Markham NAR 33: W577 (2005)), CMFinder (Yao et al BioInfo 22:445 (2006)).

“Sequence identity” is expressed as a percentage. The measurement of sequence identity of a nucleotide sequences is a method well known to those skilled in the art, using computer implementated mathematical algorithms such as ALIGN (Version 2.0), GAP, BESTFIT, BLAST (Altschul et al J. Mol. Biol. 215: 403 (1990)), FASTA and TFASTA (Wisconsin Genetic Software Package Version 8, available from Genetics Computer Group, Accelrys Inc. San Diego, Calif.), and CLUSTAL (Higgins et al, Gene 73: 237-244 (1998)), using default parameters.

Nucleic acid molecules defined herein as having sequence identity with a reference sequence may alternatively be defined as being capable of hybridising under stringent conditions to the complement of the reference sequence. Stringent hybridisation conditions are defined as those conditions under which a nucleotide sequence will preferentially hybridize to a target sequence. Increasing the stringency of the hybridisation conditions enables sequences of higher sequence identity to be found. Typical hybridisation conditions are 30-60° C., pH 7.0 to 8.3 and a salt concentration of less than 1.5 M Na⁺ ions. Preferred stringent hybridisation conditions hybridisation in 1M NaCl, 1% SDS at 37° C., and 50% formamide and washing in 0.1×SSC at 60 to 65° C.

“Structurally linked” herein means linked by a nucleic acid residue or protein or other component, so that they form part of the same structural entity, although they are not necessarily contiguous (i.e. adjacent) to one another in the structure.

“same or different riboswitches”—by the same means that they are the same riboswitch, from the same family and of the same subtype of that family, having substantially the same sequence and structure, and being able to mediate substantially the same regulatory effect on a coding sequence to which they are operably linked. Different riboswitches may be from different families or may be from the same family but be of different subtypes.

“heterologous” sequences are sequences which in nature are not operably linked to each other and/or are not found next to each other in a native sequence. In contrast, homologous sequences refer to sequences which share sequence similarity, which may be described as sequence homology. Homology is usually in a fragment of the sequence, typically in a functional domain of the sequence.

The term “substituted” herein includes all possible substituents or organic compounds. Preferably, the substituents include acyclic and cyclic, branched and unbranched, carbocyclic and heterocyclic, and aromatic and non-aromatic substituents of organic compounds. Preferred substituents include, for example, those described below. The substituents can be one or more and the same or different for appropriate organic compounds. In the present invention, the heteroatoms, such as nitrogen, can have hydrogen substituents and/or any permissible substituents of organic compounds described herein which satisfy the valences of the heteroatoms. In addition, the terms “substitution” or “substituted with” include the possibility that such substitution is in accordance with permitted valence of the substituted atom and the substituent, and that the substitution results in a stable compound, e.g. a compound that does not spontaneously undergo transformation such as by rearrangement, cyclization, elimination, etc.

“R¹,” “R²,” “R³′”, “R⁴”, “R⁵′ “R⁶′ “R⁷′ X, Y and Z herein are generic symbols to represent various specific substituents. These symbols can be any substituent, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl heoptyl octyl, nonyl, decyl, dedecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkynyl, aryl, heteroaryl, aldehyde, amino, carboxylic acid, ester, ether, halide, hydroxy, ketone, sulfo-oxo, sulfonyl, sulfone, sulfoxide, or thiol, as described below. The term “lower alkyl” is an alkyl group with 6 or fewer carbon atoms, e.g., methyl, ethyl, propyl, isopropyl, butlyl, sec-butyl, iso-butyl, tert-butyl, pentyl, hexyl, and the like.

Herein “alkyl” is generally used to refer to both unsubstitued alkyl groups and substituted alkyl groups; however, substituted alkyl groups are also specifically referred to herein by identifying the specific substituents(s) on the alkyl group. For example, the term “halogented alkyl” specifically refers to an alkyl group that is substituted with one or more halide, e.g., fluorine, chlorine, bromine, or iodine. The term “alkoxyalkyl” specifically refers to an alkyl group that is substituted with one of more alkoxy groups, as described below. The term “alkylamino” specifically refers to an alkyl group that is substituted with one of more amino groups, as described below, and the like. When “alkyl” is used in one instance and a specific term such as “halogenated alkyl” is used in another, it is not meant to imply that the term “alkyll” does not also refer to specific terms such as “halogenated alkyl” and the like.

This is the same for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., a “alkylcycloalkyl”. Similarly, a substituted alkoxy can be specifically referred to as, e.g. an “halogenated alkoxy,” a particular substituted alkenyl can be, e.g., an “alkenylalcohol”, and the like. Again, the practice of using a general term, such as “cycloalkyl”, and a specific term, such as “alkylcycloalkyl”, is not meant to imply that the general term does not also include the specific term.

The term “aryl” as used herein is a group that contains any carbon-based aromatic group including, but not limited to, benzene, naphthalene, phenyl, biphenyl, phenoxybenzene, and the like. The term “aryl” also includes “heteroaryl”, which is defined as a group that contains an aromatic group that has at least one heteroatom incorporated within the ring of the aromatic group. Examples of heteroatoms include, but are not limited to, nitrogen, oxygen, sulphur and phosphorus. Likewise, the term “non-heteroaryl”, which is also included in the term “aryl”, defines a group that contains an aromatic group that does not contain a heteroatom. The aryl group can be substituted or unsubstituted. The aryl group can be substituted with one or more groups including, but not limited to alkyl, halogenated alkyl, alkoxy, alkenyl, aryl, heteroaryl, aledhyde, amino, carboxylic acid, ester, ether, halide, hydroxyl, ketone, sulfo-oxo, sulfonyl, sulfone, sulfoxide, or thiol as described herein. The term “biaryl” is a specific type of aryl group and is included in the definition of aryl. Biaryl refers to two aryl groups that are bound together via one or more carbon-carbon bonds, as in biphenyl.

The term “cylcoalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl etc. The term “heterocycloalkyl” is a cycloalkyl group as defined above where at least one of the carbon atoms of the ring is substituted with a heteroatom such as, but not limited, to nitrogen, oxygen, sulphur or phosphorus. The cycloalkyl group and the heterocycloalkyl group can be substituted with one or more groups including, but not limited to alkyl alkoxy, alkenyl, alkenyl, aryl, heteroaryl, aldehyde, amino, carboxylic acid, ester, ether, halide, hydroxy, ketone, sulfo-oxo, sulfonyl, sulfone, sulfoxide, or thiol as described herein.

The term “cyclic group” is used herein to refer to either aryl groups, non-aryl groups (i.e. cylcoalkyl, heterocycloalkyl, cycloalkenyl, and heterocycloalkenyl groups) or both. Cyclic groups have one or more ring systems that can be substituted or unsubstituted. A cyclic group can contain one or more aryl groups, one or more non-aryl groups or one or more aryl groups and one or more non-aryl groups.

### Riboswitches

Riboswitches in the present invention include isolated, purified and recombinant riboswitches. They may be naturally occurring riboswitches, or derived therefrom, or may be artificially created, engineered riboswitches. A naturally occurring riboswitch is one which is found in nature, typically from a bacteria, eukaryote or archea, and preferably has been isolated or purified therefrom. It may be an isolated, purified or recombinant form of a riboswitch found in nature. This means that it has been extracted from a naturally occurring source, and retains the same sequence, structure and function as the riboswitch found in nature, or if recombinant, it is the same riboswitch as found in nature, but has been derived from a new genetic source.

A riboswitch which is derived from a naturally occurring riboswitch may have modified sequence, structure or function to the naturally occurring riboswitch, but preferably retains substantially the same biological or functional activity (e.g. in terms of dynamic range and output). Modified sequence means preferably a derivative of a naturally occurring riboswitch which has at least 50%, 60%, 70%, 75%, 80%, 85%, 90%, 95%, 96%, 97%, 98% or 99% sequence identity to the naturally occurring riboswitch, over a region of 20, 30, 40, 50, 100, 200, 300, 400, or 500, up to the full length of the naturally occurring riboswitch. “Modified structure” means that it retains substantially the same structure as the naturally occurring riboswitch, but may be modified, for example, in terms of the effectiveness or result of ligand binding, degree of interaction and effect on the expression platform, or degree of gene regulation upon ligand binding. By substantially the same means that the riboswitches ability to bind a particular ligand is not changed, although its affinity for the ligand may be increased or decreased. Similarly, the downstream effect of the riboswitch remains the same in terms of its ability to fold and regulate coding sequence expression, but the parameters within which this is achieved may be altered. Thus, preferably it retains substantially the same binding properties and gene regulation activity as the native riboswitch. Suitable folding packages for determining secondary structure include RNA Vienna (Hofacker NAR 1: 3429 (2003)), mfold (Zuker NAR 31: 3406 (2003), DINAMeIt (Markham NAR 33: W577 (2005)), CMFinder (Yao et al BioInfo 22:445 (2006)).

Also provided are engineered riboswitches. Engineered riboswitches have sequence alterations in terms of substitutions, additions or deletions which serve to affect their ability to bind natural, native or intracellular ligands and also their ability to bind non-natural, non-native and/or synthetic ligands. Preferably, they have a modified structure in terms of the effectiveness or result of ligand binding, degree of interaction and effect on the expression platform, or degree of gene regulation upon ligand binding. Engineered riboswitches preferably also show altered dynamic range, or output compared to the native riboswitch from which they are derived. Preferably, engineered riboswitches are orthogonal. They may have an increased or decreased dynamic range compared to the native riboswitch from which they are derived. Engineered riboswitches include, for example, riboswitches which include the consensus sequences of native riboswitches, but the remainder of the riboswitch (the non-consensus portions) have been altered. Preferably, engineered riboswitches retain at least 50%, 60%, 70%, 80%, 90%, 95%, 96%, 97%, 98% or 99% sequence identity over a region of 50%, 60%, 70%, 80%, 90%, 95%, 96%, 97%, 98% or 99% of contiguous nucleic acid residues of a consensus sequences of the native riboswitch, and/or has at least 50%, 60%, 70%, 80%, 90%, 95%, 96%, 97%, 98% or 99% similarity in terms of structure with the consensus sequence of the native riboswitch, when measured using packages such as those defined above.

Also included are chimeric riboswitches, which may comprise portions, domains or functionality from two or more riboswitches. For example, the aptamer domain may be provided from a first riboswitch, and the regulatory domain or expression platform from a second riboswitch. Chimeric riboswitches may also be consensus riboswitches, in that each domain may comprise the consensus sequence of the domain of the riboswitch from which is obtained. Preferably, chimeric riboswitches comprise part of a riboswitch from a riboswitch of a particular class or type, and part of a riboswitch from the same class or type of riboswitch, or part of a riboswitch from a different class or type of riboswitch. Also included within the present invention are chimeric riboswitches which comprise part of a riboswitch and non-riboswitch sequence or parts. Also envisaged is that an aptamer domain and/or an expression platform may be non-riboswitch derived (i.e. may come from another source), and when placed with a heterologous aptamer domain or expression platform, forms a chimeric molecule which functions as a riboswitch. Each domain (i.e. aptamer domain or expression platform) may have a native structure, and therefore native activity in terms of effectiveness or result of ligand binding, degree of interaction and effect on the expression platform, or degree of gene regulation upon ligand binding Alternatively, the combination of domains in a chimeric riboswitch may exhibit altered effectiveness of ligand binding, degree of interaction and effect on the expression platform, or degree of gene regulation upon ligand binding. They may have an altered dynamic range, or output compared to the native riboswitch from which they are derived. Chimeric riboswitches may also be engineered. Preferably, chimeric riboswitches are orthogonal.

The riboswitch may comprise one or more (e.g. 2, 3, 4, 5, or 6 or more) aptamer domains. Where two or more aptamer domains are present in a riboswitch, these may exhibit cooperative binding of ligands, or independent binding of ligands. The two or more aptamer domains may be the same or different, and may bind the same or different ligands to one another.

Also envisaged is a riboswitch comprising a single aptamer domain which is capable of binding two or more ligands simultaneously, which in some scenarios may be necessary to mediate the conformational change required to regulate coding sequence expression. Thus, reference herein to a ligand includes reference to the combined use of two or more ligands simultaneously.

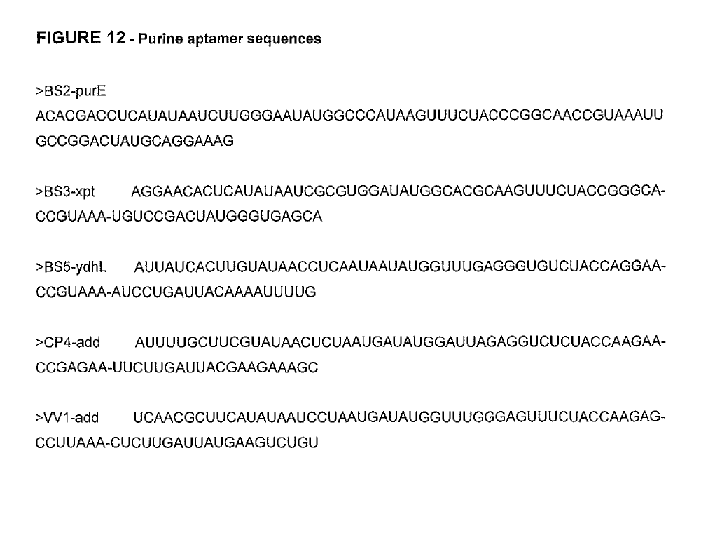

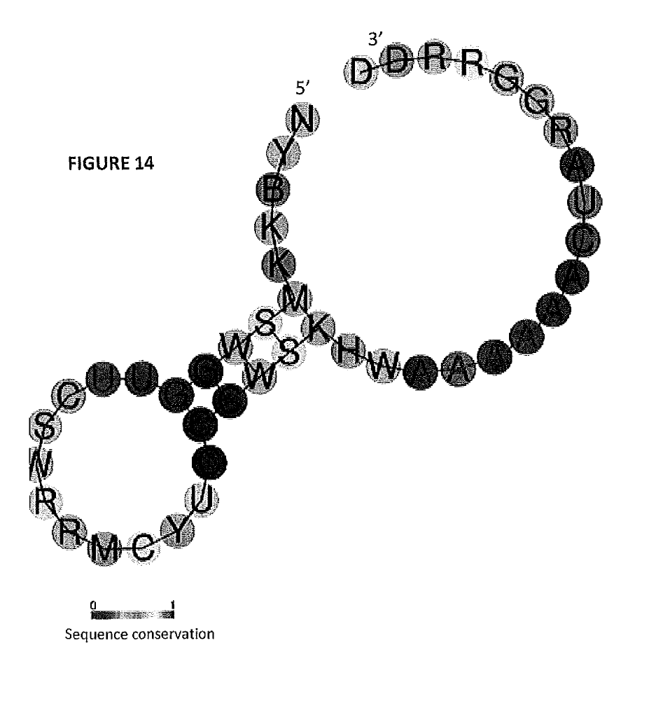

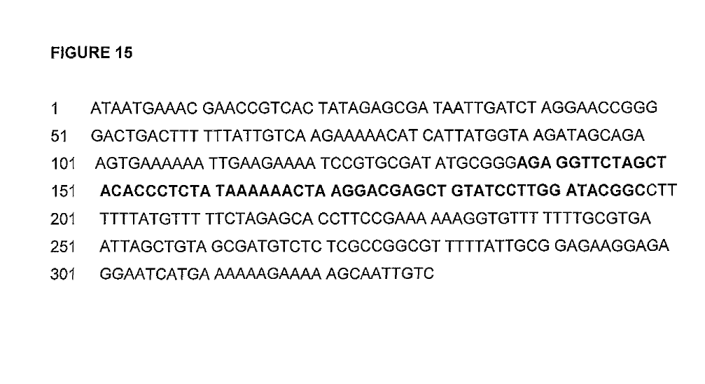

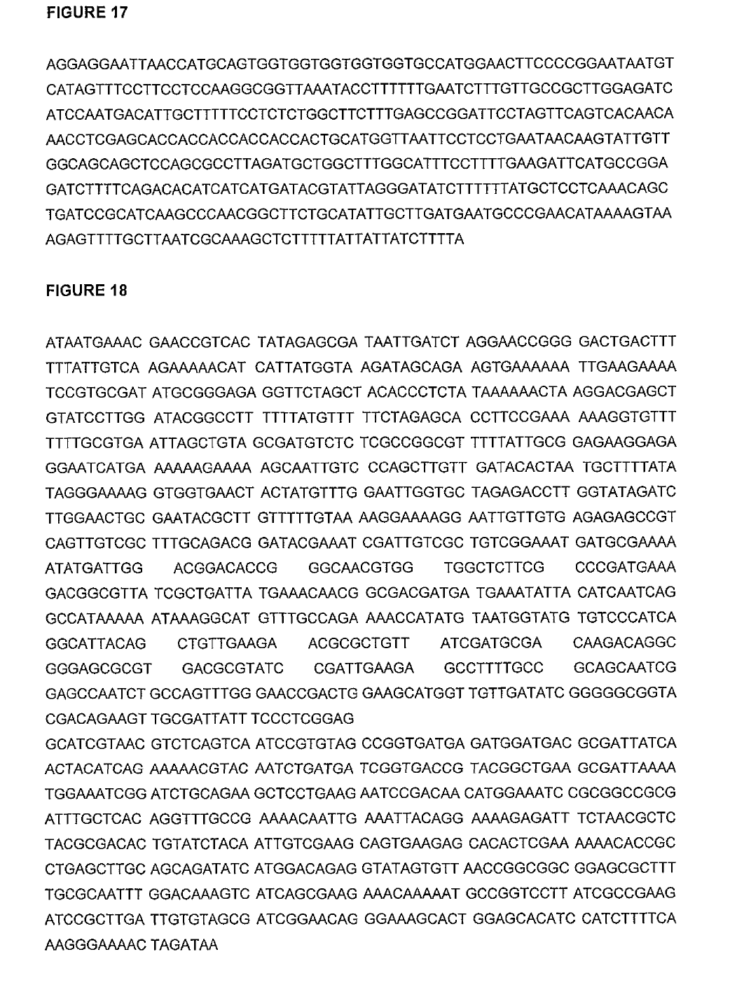

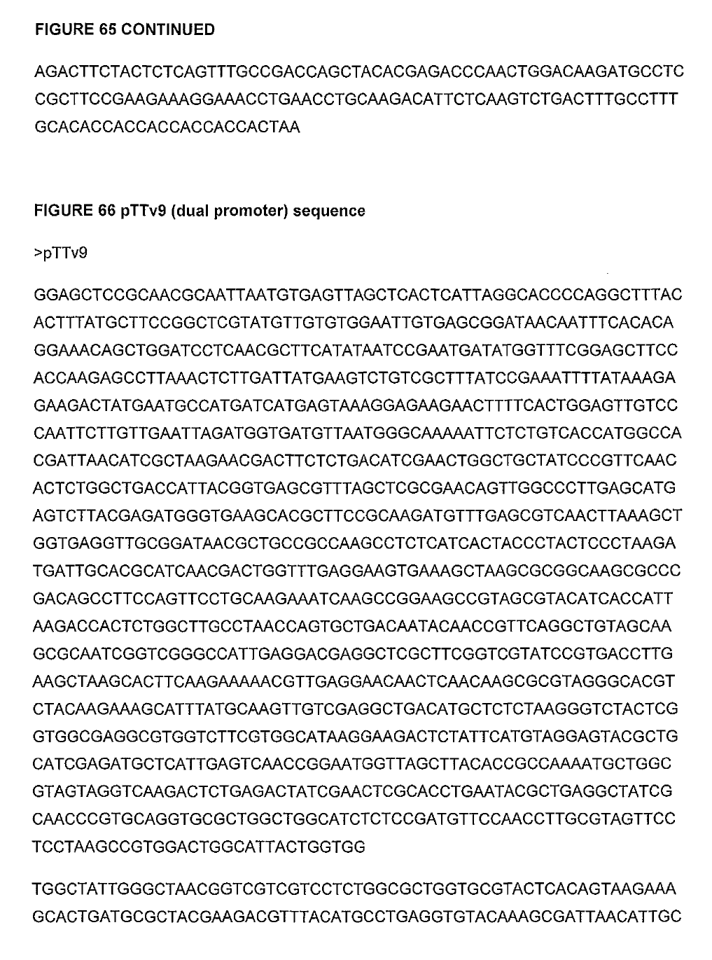

Preferred riboswitches for use in the present invention include purine riboswitches, for example those of FIG. 11, an Add-A riboswitch or one which is derived therefrom, such as the M6, M6′, M6″ or M6C, M6C′ or M6C″ such as those of FIG. 13 or FIG. 62 (Dixon et al PNAS 107: 2830 (2010)), or a PreQ riboswitch such as that of FIG. 14 or a PreQ mutant consensus sequence such as that of FIG. 16.

A riboswitch used in the present invention may be controlled by any suitable ligand. Suitable ligands are defined herein, and many examples are known to persons skilled in the art. Binding of a ligand to a riboswitch may cause activation (i.e. cause a conformational change in the expression platform leading to up-regulation of coding sequence expression); repression (i.e. cause a conformational change in the expression platform leading to down-regulation of coding sequence expression); inhibition (i.e. prevention or reduction in the conformational change of the expression platform, leading to an inhibition of the riboswitch mediated regulation of coding sequence expression) and blocking of the riboswitch (i.e. prevention of any conformational change in the expression platform). The effect of the ligand on the riboswitch is non-constitutive.

Preferably, the effect of the ligand on the riboswitch takes place when the ligand reaches a threshold level in the expression system or host cell comprising the genetic construct of the invention.

The threshold level for a particular ligand will be the specific concentration within the expression system or host cell at which the riboswitch undergoes a conformational change. For any particular ligand/riboswitch combination, a threshold level can be determined by a person skilled in the art using standard methodology. Such methodology can be used to then produce a standard dose-expression curve for any particular ligand and riboswitch combination.

### Aptamer Domain

The aptamer domain may be a naturally occurring aptamer domain, or may be derived, or re-engineered therefrom or artificially created. It may be isolated, purified or recombinant. Alternatively, it may not be derived from a native riboswitch, but may be a ligand binding domain of an RNA molecule, which when operably linked to an expression platform is capable of acting as an aptamer domain of a riboswitch.

The aptamer domain may have dual functionality, meaning that it is able to bind a ligand which mediates an effect on the conformational change in the expression platform (i.e. an activation or an inhibition or a blocking thereof), and also upon binding is able to mediate other downstream/upstream effects, such as control of other RNA molecules, stabilisation of mRNA, protection from mRNA degradation nuclease or chemical, or other genetic switches.

The aptamer domain may comprise a consensus sequence, which is a motif or common sequence shared by a particular type or class of riboswitches or aptamer domains, for example dictated by the class or type of ligands bound by the aptamer domain. Consensus sequences for aptamer domains of riboswitches will be known in the art.

The aptamer domain of a riboswitch of the invention may be chimeric, for example comprising a portion of an aptamer domain of one riboswitch, and a portion of an aptamer domain of one or more different riboswitches. In this way, different functionalities such as preference for ligand binding and effect on the expression platform can be engineered into a particular aptamer domain. Preferably, such aptamer domains will comprise at least a consensus sequence for a ligand binding portion of the aptamer domain, or a sequence which is derived therefrom and has at least 50, 60, 70, 80, 85, 90, 95, 96, 97, 98, or 99% sequence identity over at least 50, 60, 70, 80, 85, 90, 95, 96, 97, 98 or 99% of the length of the consensus sequence.

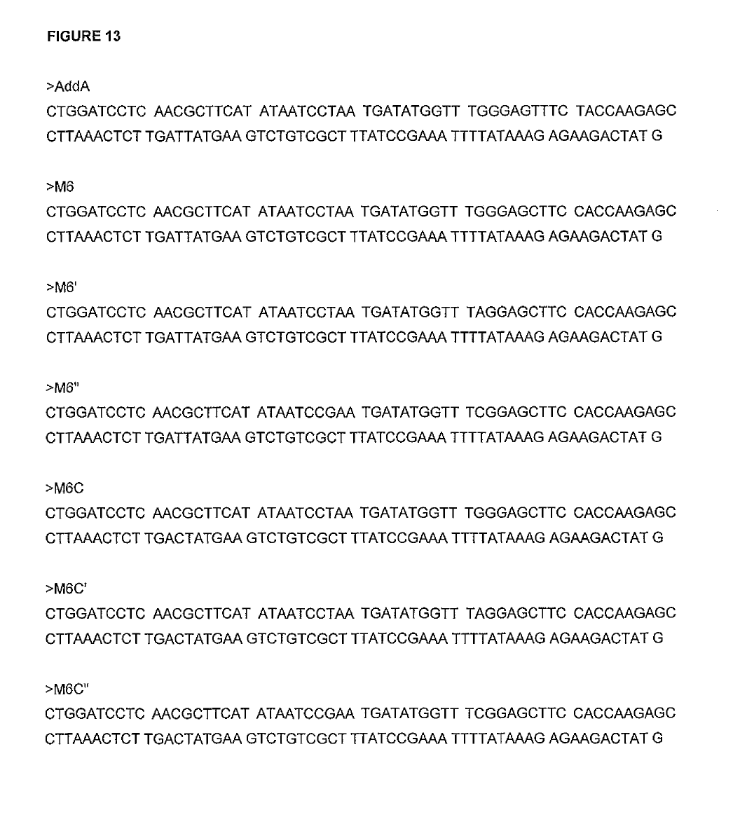

Preferred aptamer domains include those derived from purine riboswitches, for example those of FIG. 12; those of an Add-A riboswitch or a riboswitch derived therefrom such as the M6, M6′, M6″ or M6C, M6C′ or M6C″ (Dixon et al PNAS 107: 2830 (2010)) riboswitches of FIG. 13; or those of a PreQ riboswitch such as that of FIG. 14 (Roth, Breaker. Annu Rev Biochem 78:305 (2009), Barrick, Breaker Genome Biol 8:R239 (2007)). Specific aptamer domains preferred for use in the present invention are the purine aptamer domains of FIG. 12.

The aptamer domain is preferably operably linked to the expression platform of the riboswitch. Preferably, it is structurally linked, forming party of the same nucleic acid molecule. Preferably, it is linked thereto by a nucleic acid molecule linker, which is preferably an RNA coding sequence, such that when transcribed the aptamer domain and expression platform remain structurally linked by a nucleic acid molecule linker.

The riboswitches of the present invention include those comprising two or more aptamer domains operably linked to a single expression platform. The aptamer domains may be the same or different. The effect on the conformational change of the expression platform may be dictated by the binding of one, the other, or both of the ligands. The ligands may be the same or different. At least one of the aptamer domains and the expression platform are heterologous.

### Expression Platform Domain

The expression platform is a nucleic acid sequence, preferably an RNA sequence. The expression platform may be a naturally occurring expression platform, or may be derived, re-engineered therefrom or artificially created. It may be isolated, purified or recombinant. It may not be derived from a native riboswitch, but may be a regulatory molecule which is able to adopt at least two different configurations, each of which being able to mediate a different (either inhibitory or activating) effect on expression of a nucleic acid sequence to which it is linked. Preferably, it is able to be change configuration upon binding of a ligand to an aptamer domain, to which the expression platform is operably, and preferably structurally, linked.

The expression platform may comprise a consensus sequence, which is a motif or common sequence shared by a particular type or class of riboswitches or expression platforms, for example dictated by the class or type of ligands bound by the expression platforms. Consensus sequences for expression platforms of particular riboswitches are known in the art for example, pbuE (ydhL), xptG, PreQ and addA.

The expression platform of a riboswitch of the invention may be chimeric, for example comprising a portion of an expression platform of one riboswitch, and a portion of an expression platform of one or more different riboswitches. In this way, different functionalities such as manner of regulating gene expression and folding, may be achieved. Preferably, such an expression platform will comprise at least a consensus sequence of the expression platform, or a sequence which is derived therefrom and has at least 50, 60, 70, 80, 85, 90, 95, 96, 97, 98, or 99% sequence identity over at least 50, 60, 70, 80, 85, 90, 95, 96, 97, 98 or 99% of the length of the consensus sequence.

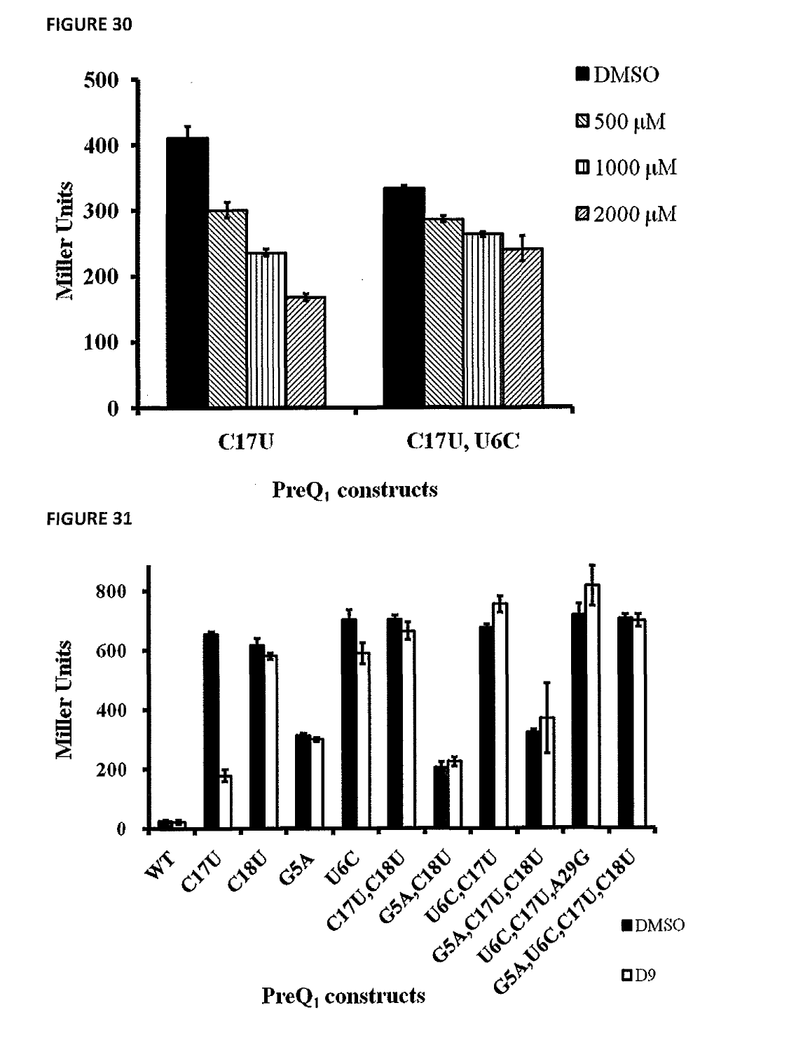

Preferred expression platforms include those derived from FIG. 27, an Add-A riboswitch such as that of FIG. 13, or one which is derived therefrom, such as the M6, M6′, M6″ or M6C, M6C′ or M6C″ (Dixon et al Proc Natl Acad Sci USA 2010, 107, 2830), or a PreQ riboswitch such as that of FIG. 14. (Roth, Breaker. Annu Rev Biochem 78:305 (2009), Barrick, Breaker Genome Biol 8:R239 (2007)). Specific expression platforms preferred for use in the present invention are the expression platforms of pbuE, xpt, ydhL, PreQ addA riboswitches.

The expression platform is preferably operably linked to the aptamer domain of the riboswitch. Preferably, it is structurally linked, forming part of the same nucleic acid molecule.

Chimeric riboswitches comprising heterologous aptamer domain and expression platforms may be produced using standard methodology known in the art, for example as described in Sambook et al 2000 Molecular Cloning; A Laboratory Manual (3^{rd} edition).

### Spacer

Preferably, the spacer used in the genetic construct of the invention is a nucleic acid molecule which separates the riboswitch and the gene of interest, and preferably serves to enhance the ability of the riboswitch to regulate gene expression. Preferably, the spacer modulates the expression of a coding sequence when linked to the riboswitch, as compared to the regulation of expression when the coding sequence is regulated by the riboswitch in the absence of a spacer. By modulation means an increase or decrease in the output of the coding sequence, i.e. in terms of the yield of RNA transcripts or protein or peptide. Preferably, the spacer serves to increase the output of the coding sequence, in terms of RNA transcripts or protein or peptide yielded as compared to output when regulated by the same riboswitch and regulatory elements but no spacer.

The inventors have surprisingly found that the presence of a spacer in a genetic construct in accordance with the first aspect of the invention, as compared to an otherwise identical control genetic construct from which a spacer is absent, is able to markedly increase both the absolute amount of expression of a gene of interest (encoded by the coding sequence of the second nucleic acid molecule), and also to increase the ratio of expression levels in the presence or absence of the riboswitch's ligand. Thus the presence of the spacer sequence is able to increase the dynamic window of induction exhibited by a genetic construct in accordance with the present invention.

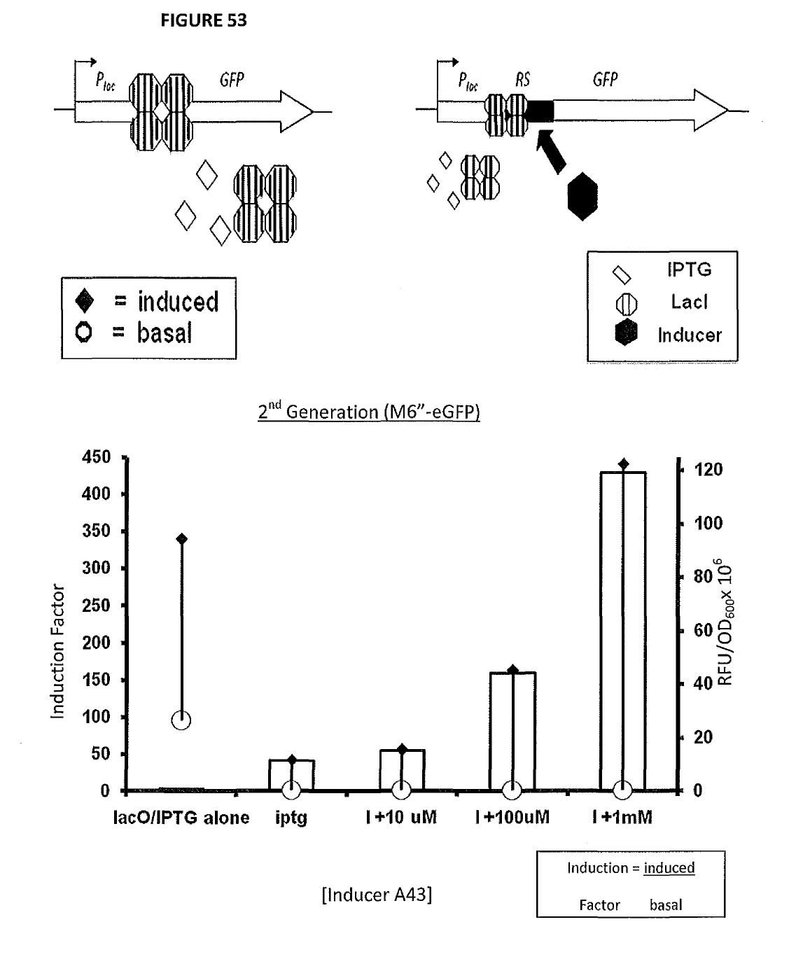

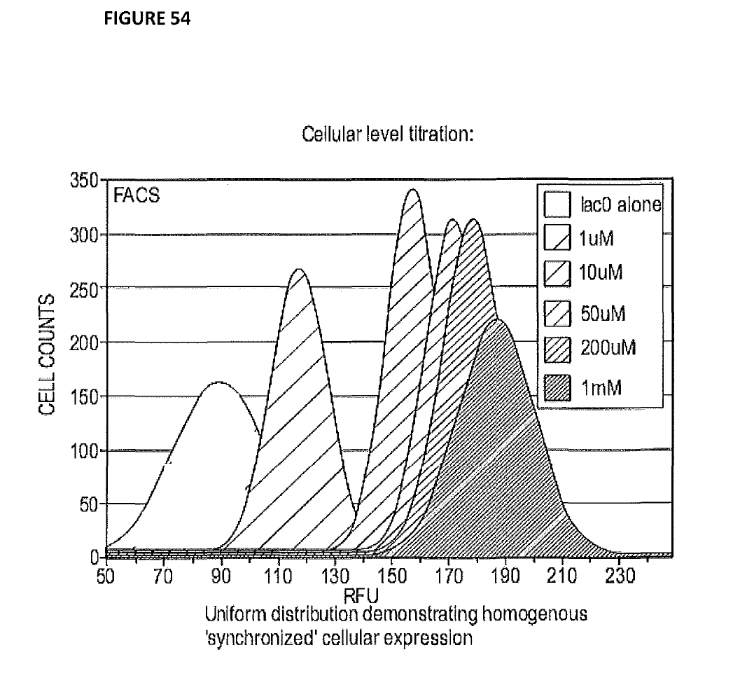

This is illustrated in FIGS. 43, 44, and 45, which illustrate differences in induction of various genes of interest (respectively lacZ, DsRed and T7 RNA polymerase) by riboswitches in genetic constructs in which a spacer is in which a spacer sequence is present (constituting examples of genetic constructs in accordance with the present invention), or from which a spacer sequence is absent (these genetic constructs constituting control constructs used for comparison). In each of these examples the spacer sequences utilised were 105 nucleotides in length.

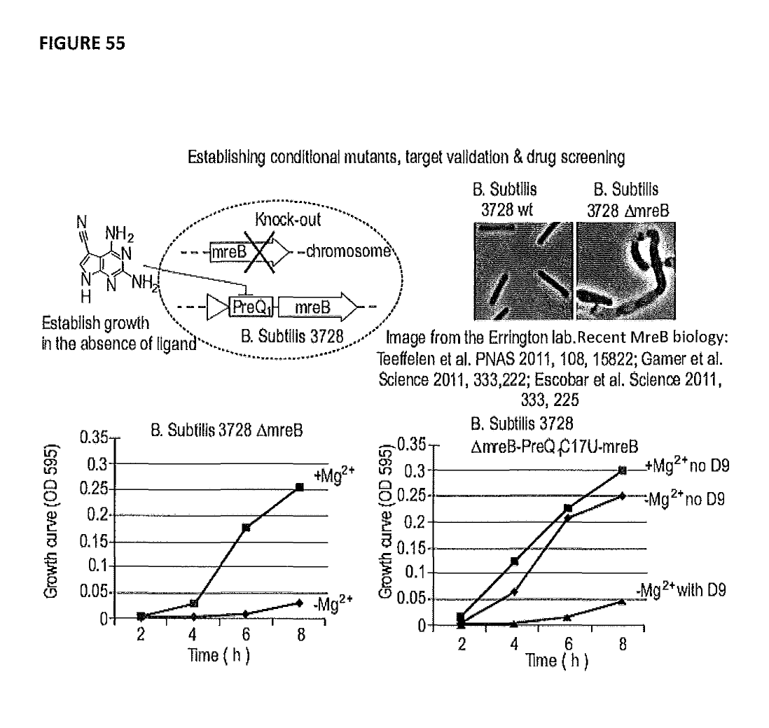

It can be seen from each of these Figures that the presence of a spacer sequence in the genetic construct leads to an increased dynamic range exhibited by the genetic constructs of the invention as opposed to the control constructs. Furthermore, as can readily be seen from FIG. 45, in genetic constructs of the invention comprising a suitable spacer sequence a exhibit a dose-dependent increase in induction of the gene of interest in response to the presence of the riboswitch's ligand, as compared to the “binary” response of the control constructs (in which presence of the ligand gives rise to expression of the gene of interest independent of the amount of the ligand present). The difference between the dose dependent and binary induction is clearly illustrated in the blot data presented in FIG. 45.

As defined above, a spacer preferably has a low GC content. The GC content of a nucleic acid is the percentage of nitrogenous bases on a nucleic acid molecule that are either guanine or cytosine.

The GC content may be calculated as

$$\frac{G + C}{A + T + G + C} \times 100$$

whereas the AT/GC ratio is calculated as

$$\frac{A + T}{G + C} .$$

The GC-content percentages as well as GC-ratio can be measured by several means, preferably by measuring the melting temperature of the nucleic acid sequence by spectrophotometry. The absorbance of DNA at a wavelength of 260 nm increases fairly sharply when the double-stranded DNA separates into two single strands when sufficiently heated. Another suitable method for non-sequenced nucleic acid molecules uses flow cytometry for large number of samples.

In alternative manner, if the DNA or RNA molecule under investigation has been sequenced then the GC-content can be accurately calculated by using the above formula.

Preferably, the spacer is positioned upstream of the gene of interest. Preferably, the spacer is positioned upstream of the gene of interest. Preferably, it may be immediately upstream, such that the last nucleic acid residue of the spacer is adjacent to the first nucleic acid residue of the start codon of the gene of interest. Alternatively, the spacer may be separated from the start codon of the gene of interest by a non-coding and/or non-functional sequence. Alternatively or additionally, the spacer may be separated from the gene of interest by one or more upstream sequences such as additional regulatory sequences such as transcriptional or translational regulatory elements, additional coding or non coding sequences, ribosome binding sites, additional riboswitches, chromosomal integration sites, and transcription terminators, marker genes, reporter genes, coding sequences for ancillary regulators, repressors, and any other desired components, many of which will be known to persons skilled in the art.

Where two or more riboswitches are present regulating a nucleic acid molecule comprising a coding sequence, then a spacer may be provided upstream of the nucleic acid molecule comprising the coding sequence, and downstream of the riboswitches.

The spacer may be any size or sequence which serves to have the effect of enhancing regulation of the gene of interest by the riboswitch, preferably to increase or decrease the dynamic range of the riboswitch, or the output of the coding sequence in terms of yield of RNA transcripts or protein or peptide. The spacer is preferably a DNA molecule, which encodes an RNA molecule and which is operably linked to the RNA molecule which encodes the riboswitch, which in turn may preferably be operably linked to the gene of interest under its regulation. The spacer may be a coding sequence or a non-coding sequence, or a combination thereof. The spacer may be a naturally occurring sequence, or an artificial sequence. It may be isolated or purified from a sequence which exists in nature, or may be recombinantly produced, which means that it is derived from a new genetic source rather than being derived from a native sequence. The spacer may be naturally associated with the riboswitch i.e. it is operably linked thereto in nature, or may be heterologous to the riboswitch.

The spacer may be 5, 20, 15, 20, 25, 30, 35, 40, 45, 50, 55, 60, 65, 70, 75, 80, 85, 90, 95, 100, 105, 110, 120, 125, 130, 135, 140, 145, 150, 200, 250, 300 or more nucleotides in length. It will be appreciated that in order that nucleic acid molecules connected by a spacer may be kept in frame, the number of nucleotides incorporated in a spacer should preferably be a multiple of three. In keeping with this, the spacer may suitably be 5, 20, 15, 20, 25, 30, 35, 40, 45, 50, 55, 60, 65, 70, 75, 80, 85, 90, 95, 100, 105, 110, 120, 125, 130, 135, 140, 145, 150, 200, 250, 300 or more nucleotides in length.

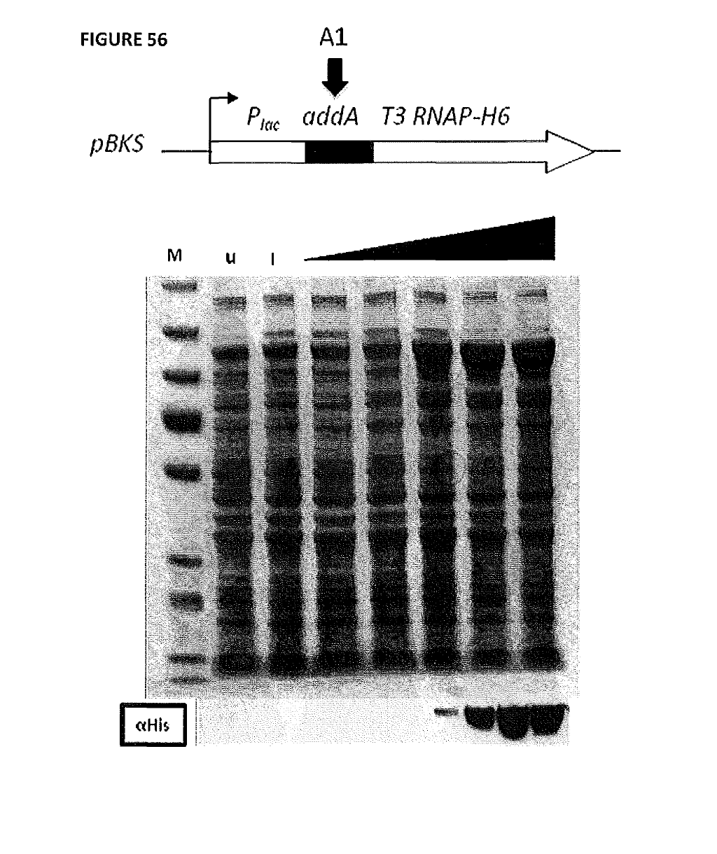

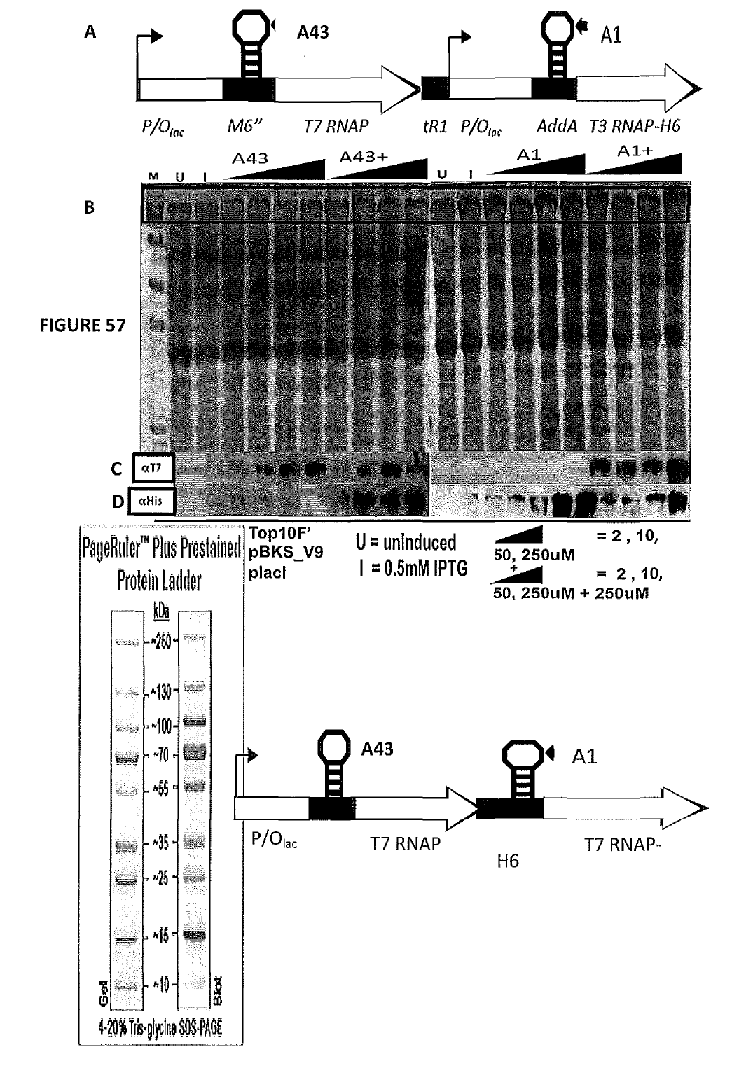

More information regarding the impact of the length of spacer sequence on the ability to bring about dose-responsive induction of a gene of interest is shown in FIGS. 46 and 47, both of which report on induction of T7 RNA polymerase under riboswitch control in genetic constructs of the invention incorporating reporter sequences of different lengths.

From FIGS. 46 and 47 it can be seen that dose-dependent induction is seen when spacer sequence length varies between about 90 nucleotides and about 165 nucleotides. Below 90 nucleotides spacer length the dynamic range of expression upon the addition of 250 uM A43 plus 0.5 mM IPTG is <2fold (above the expression induced by IPTG alone), for constructs with spacer lengths of 90 nucleotides and above this value is generally >2 fold.

These graphs show the regulation afforded upon the addition of IPTG (0.5 mM) varies for constructs of different spacer length from ˜1-fold for sp120, to 44-fold for sp105. Upon addition of the riboswitch inducer A43 the induction factors above IPTG expression again vary greatly for constructs with different lengths, with sp120 exhibiting up to 12-fold regulation. Overall the construct with 105 nt spacer length permits the biggest dynamic range of control (max/un-induced) of 134-fold, upon the addition of IPTG (0.5 mM) plus A43 (250 uM).

Accordingly it will be recognised that a preferred length of the spacer is 105 nucleotides in length. As referred to above, the inventors have found that use of a spacer sequence of around 105 nucleotides length confers a number of advantages, which may include increased dynamic range in response to the riboswitch ligand, and dose-dependent induction of the gene of interest.

Preferably, the spacer is derived from a naturally occurring coding sequence, which may be eukaryotic or non-eukaryotic. Preferably it is derived from a nucleotide sequence which encodes a protein or polypeptide. It may be derived from the coding sequence thereof, including from exons or introns of said coding sequence, or from an upstream sequence associated with the coding sequence or from a downstream region. It may be a translated, coding sequence or a non-translated sequence. It may comprise a combination, either naturally occurring or artificially created, of coding and non-coding sequence regulatory and non-regulatory, upstream, coding and downstream sequence, or sequences from heterologous sources.

Preferably, it is derived from a protein, preferably a non-bacterial protein, preferably a eukaryotic protein. Preferably, it is derived from a marker protein, for example enhanced green fluorescent protein (eGFP), or DsRed. Other examples include T7, T3 or SP6.

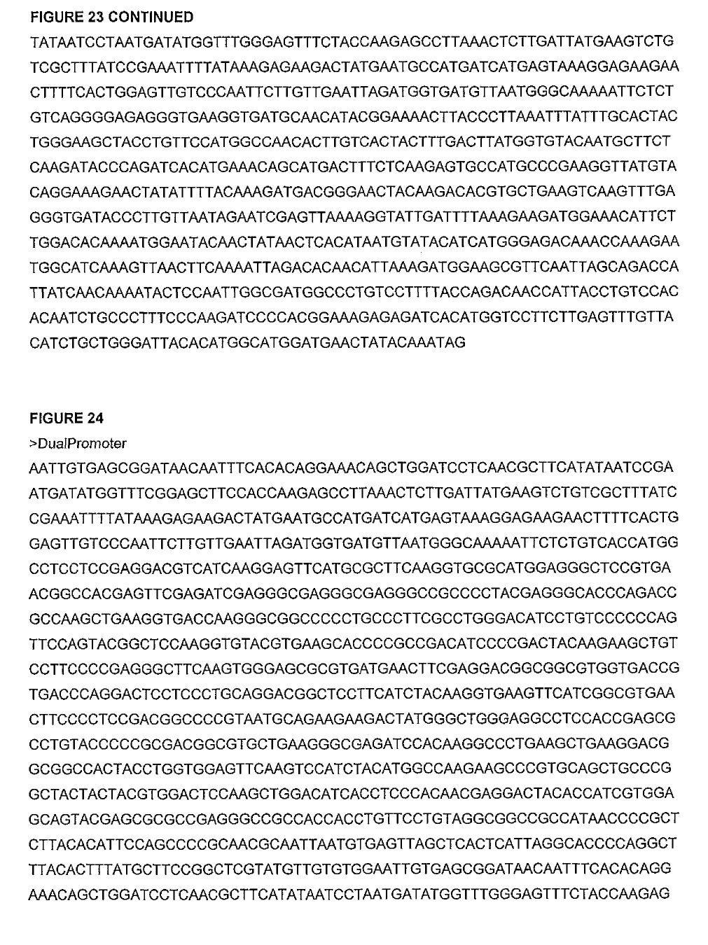

Preferred spacers for use in the present invention are all or part of a spacer sequence provided in FIG. 19 or a sequence which is derived therefrom. Thus, a preferred spacer sequence for use in the present invention may be a spacer of GFP 165, GFP 150, GFP 135, GFP 120, GFP 105, GFP 102, GFP 99, GFP 96, GFP 93, GFP_{—}90, GFP_{—}75, GFP_{—}60, GFP_{—}45, GFP_{—}30, GFP_codon optimized variant, DSRed, lacZ, T7, T7, T3 or SPG as shown in FIG. 19, or fragments thereof. Preferably, a fragment thereof will comprise at least 6, 9, 12, 15, 30, 45, 60, 75, 90 or 99 contiguous nucleotides of a sequence of FIG. 19. A spacer may comprise a sequence which has at least 50, 60, 70, 80, 85, 90, 95, 96, 97, 98 or 99% sequence identity across at least 10, 20, 30, 40, 50, 60, 70, 80, 85, 90, 95, 96, 97, 98 or 99% of the sequence from which it is derived, that sequence preferably being a sequence of FIG. 19 or a fragment thereof.

Preferably, the spacer will be heterologous to the genetic construct in which it is placed, and is distinguished therefrom by its ability to moderate the function and activity of the riboswitch with which it is associated.

### Genetic Construct

The genetic construct of the invention may be DNA or may be RNA. Preferably, it is a DNA construct, which encodes an RNA riboswitch operably linked to a spacer and a gene of interest.

Two or more of the components of the genetic construct may be heterologous, i.e. from different sources. Thus, they may be from different RNA molecules, different transcripts, different genes, different cells, different organisms or different species. When heterologous, they may each independently be naturally occurring, artificial or engineered sequences, and/or independently isolated, purified or recombinant.

Preferably, a genetic construct may comprise one or more transcriptional or translational regulatory elements, additional coding or non coding sequences, ribosome binding sites, chromosomal integration sites, and transcription terminators, marker genes, reporter genes, coding sequences for ancillary regulators, repressors, and any other desired components, many of which will be known to persons skilled in the art. The position of such additional regulatory elements within the genetic construct will depend upon their role within the construct, and the nature of components or other elements or nucleic acid molecules they are operably linked to.

The genetic construct of the invention may be a vector, which is a carrier of exogenous nucleic acid. Any suitable vector may be used as a genetic construct in the present invention. The vector may be eukaryotic or prokaryotic. Vectors include plasmids, viral vectors, phage, cosmids, artificial chromosomes and any others known to persons skilled in the art. A vector may be integrative. A vector may be replicative. It may be conjugative or non-conjugative, an expression vector, cloning vector or shuttle vector.

Suitable viral vectors include (but are not limited to), for example herpes virus, Vaccinia virus, polio virus, adenovirus, adeno-associated virus, AIDS virus, HIV virus, Pox virus, and other RNA viruses. Retroviral vectors include MMLV, pBADE, pADCMV, CMV, MoMLV. Viral vectors also comprise the vectors which share sufficient properties with the viral vectors making them suitable for use as a vector. Viral vectors preferably also include early and late genes, preferably non structural early genes and structural late genes, inverted terminal repeats, and RNA polymerase transcript, and regulatory sequences such as a promoter. Preferably, one or more of the early genes of the virus may have been removed and replaced by exogenous DNA, such as the nucleic acid comprising the coding region. In such a case, any necessary early genes may be provided by the host cell or in vitro expression system, as required. Viral vectors have the advantages of being able to carry a large load of exogenous nucleic acid (for example, up to 8 kb), being thermostable, and being capable of being stored at room temperature, as well as being able to transfect non-dividing cells. Preferably, viral vectors will have been modified such that they do not induce an immune response in the host organism. Preferably, viral vectors will be replication defective. Known viral vectors will be known to persons skilled in the art.

Other suitable vectors include plant vectors, such as plant shuttle vectors.

Specifically, examples of vectors include pGEXxx, pETxx, pMODxx, pBlueScriptxx, pDGxx, pACxx, pMODxx, pBRxx, pBC Phagemid Vectors, Lambda vectors, pCMV, pCR, pTOPO, pBAD, pEM7, pRSET, pTrc, pENTR, pDEST, pxxDuet, pCDFxx, pRSFxx, pCITExx, pFLAGxx, pTACxx, pPCV, pShuttlePHT43, pMUTIN4, pAMJ2008, pACYxx, p15xx.

The vector selected for use in the present invention will be appropriate to the expression system in which it is placed, i.e. in vivo expression for example in a host cell, or in vitro expression. Where the invention uses two or more genetic constructs, these may be the same or different types, independently selected from the group consisting of plasmids, viral vectors, phage, cosmids, artificial chromosomes.

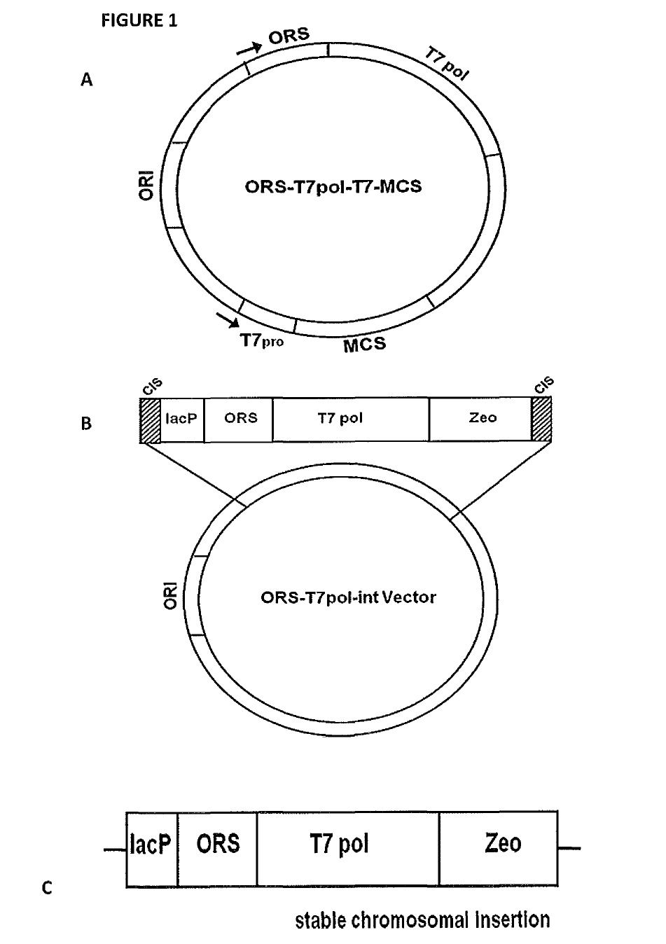

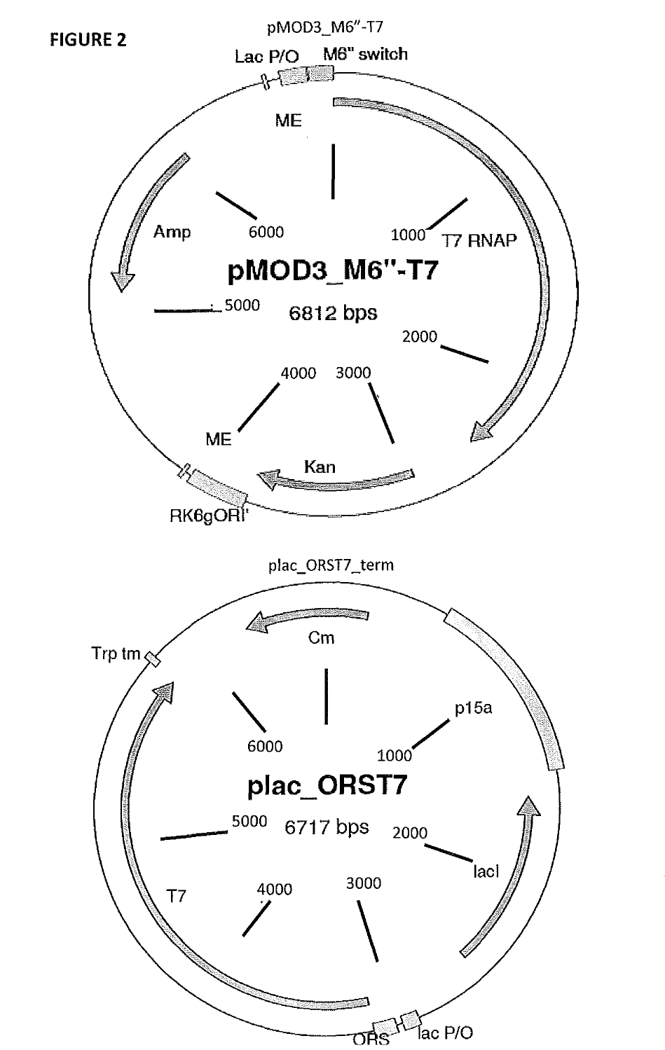

Examples of vector maps and genetic constructs are shown in FIGS. 1 and 2.

### Coding Sequence

The riboswitch of the present invention may be operably linked to and used to regulate a variety of sequences, including nucleic acid sequences which comprise coding sequences (i.e. a nucleic acid molecule which comprises codons for amino acids, preferably two or more contiguous codons for amino acids) and preferably which can be transcribed into RNA, and which in turn can preferably be transcribed into protein or peptide; and nucleic acids comprising non-coding sequences (i.e. the nucleic acid sequence does not comprise two or more contiguous codons for amino acids). The nucleic acid sequence regulated by the riboswitch may complementary to the above mentioned coding sequences (i.e. the complementary strand where the nucleic acid molecule is double stranded). The nucleic acid molecules may comprise both coding and non-coding sequences, for example many mRNA molecules include both coding sequence and upstream a functional non-coding region such as rRNA and tRNA. The nucleic acid molecules regulated by the riboswitch may be DNA, RNA, PNA, LNA, GNA or TNA.

Regulation of a nucleic acid sequence by a riboswitch includes regulation of expression, wherein expression includes transcription, and translation.

Where two or more coding sequences are present in a genetic construct, these may be the same or they may be different. By the same, means that they comprise the same nucleic acid sequence as each other, at DNA level. Different coding sequences are those which have different nucleic acid sequences at the DNA level, and so may produce different RNA transcripts and different amino acid sequences, although it is possible that the different coding sequences encode the same product having the same functional and/or activity, but differing in sequence at one or more of the DNA, RNA or amino acid levels.

Preferably, the coding sequence of a genetic construct of the invention will be heterologous to the riboswitch to which it is operably linked to. Heterologous means that in nature, the sequences are not operably linked. Thus, they may be from the same or different sources, but are not naturally associated with one another and do not naturally have any functional relationship with one another.

Preferred coding sequences for use in the present invention include antisense genes, repressors, oscillators, quorum sensing genes, transcription factors, motility genes, morphology genes, reporters, drug target homologs, and antibiotic genes. Specific examples include the genes coding for T7 RNA polymerase, T3 RNA polymerase, SP6 RNA polymerase, eGFP, LacZ and DsRed.

Preferred coding sequences include those encoding human proteins or parts thereof. Non-limiting examples of human proteins for expression in the present invention include Pim-1, Pim-2, MAPKAPK5, TRIB1, FYN, MAPK1, MAPK14, MAPK8, EMG1, IFN gamma, IFN alpha, TNF alpha, NFKB1a, YY1, TFIIB, Kif11, CDC2, Caveolin, CSF2, HGF, G-CSF, GMCSF, Interferon, IL-4, IL-6, IL-7, IL-10, IFN alpha, TNF alpha, CCL5, TFIIB and multimeric proteins including, but not limited to, Human Growth factors, erythropoietin, thrombopoietin, interferons, insulin-like growth factors, epidermal growth factors, platelet-derived growth factors, angiogenin, angiotensin, interleukins, GPCRs, taste receptors, odorant receptors, Leucine zippers, Chimeric Zinc Fingers, Heteromeric cytokines, T cell receptors, antibodies, heavy light chain, fragments, fab, fv, chemokines, and cytokines.

One of the many challenges of expressing recombinant antibodies in bacterial expression systems is the lack glycosylation machinery, use of the co-expression systems demonstrated in this invention, to precisely control the expression of eukaryotic glycosylation machinery in E. coli cells as shown possible recently (Valderrama-Rincon et al Nature Chemical Biology 2012 8, 434-436.), would allow the expression of this glycosylation machinery to be regulated to aid the challenge of expressing antibodies in bacterial expression systems such as E. coli, B. subtilis, P. fluorescens.

An additional challenge of expressing antibodies and antibody fragment such as Fabs in bacterial expression systems, is the challenge of balancing the co-expressional requirements of the antibody heavy and light chains. It is very difficult to predict expression requirements based on promoter strength, codon usage, mRNA structure prediction, and cellular copy number of the gene(s) of interest. Current methods involve a combinatorial challenge of manually changing promoter strengths, and altering the copy number of the vector(s) containing the recombinant gene(s) of interest. Additional methods involve using intergenic sequences between the genes of interest within a discistronic expression cassette, again this intergenic sequence needs to be optimized and re-selected for each co-expression requirement. The ability of the dual promoter expression and operon systems developed means that the optimal co-expression ratio(s) can be readily controlled by addition of differing inducer concentrations to the growth media. This has application for controlling the co-expression of heavy and light chains of antibodies and anti-body fragments.

### Regulatory Sequences

A regulatory sequence as used in the second aspect of the invention is a sequence which may be used to regulate or control the expression of a coding sequence, preferably provided in a second genetic construct. The regulatory sequence may encode an antisense sequence, or protein or peptide which regulates transcription or translation of a coding sequence, for example a polymerase, a transcription factor, an oscillator, a repressor, an activator, or a regulatory RNA molecule.

### Control Elements

The control elements provided in the genetic construct of the invention may be transcriptional or translational control elements, and may include promoters, enhancers, ribosome binding sites, transcription terminators, repressors and other ancillary regulators.

Promoters suitable for use in the genetic constructs of the invention will be those which can regulate transcription of the nucleic acid molecules to which it is operably linked, in the vector in which it provided. A promoter may be constitutive or inducible. It may be a positive or negative operator.

Preferably, a promoter will be one which is capable of directing transcription in the expression system of choice, and will be appropriately selected. More preferably, a promoter will be selected which is capable of functioning in a variety of expression systems or host cell types. Preferably, a promoter will be provided in close proximity to the transcription start site of the nucleic acid molecule under its control. The promoter sequence may be a naturally occurring promoter sequence, isolated or purified from any suitable source, including for example eukaryotes (such as yeast, fungi, algae, protist, insect, plant, animal or human), prokaryotes such as bacteria and archaea, viruses and organelles such as chloroplasts and mitochondria. Alternatively, the promoter sequence may be an engineered sequence, preferably derived from a naturally occurring sequence, but modified to comprise characteristics suitable for the desired expression of the nucleic acid molecule to which it is linked in the constructs of the invention.

T7, T3, Sp6, araC, araBAD, lac promoter, lacUV5, tac, trc, trpR promoter Omp, lac promoter/operator, tet promoter/operator, luxR, Lambda Prm promoter, pLac promoter.

Preferably, in each genetic construct of the invention, each nucleic acid molecule to be transcribed will be under the control of a promoter sequence. Where two or more nucleic acid molecules are operably linked, for example a riboswitch and a coding sequence, or two or more coding sequences, these may be transcribed under the control of a single promoter, preferably positioned upstream of the sequences to be transcribed. Alternatively, the two or more nucleic acid molecules of the genetic construct to be transcribed may each be under the control of individual promoters. Where more than two nucleic acid molecules within a construct are to be transcribed, two or more of these may be under the control of the same or different promoters. Where two or more promoters are present in a genetic construct of the invention, these may be the same promoter, or may be derived from the same source, or of the same type, or may be different. It may be desirable to use different promoters where different levels of regulation are required, for example, responsiveness to different transcription factors, environmental conditions such as temperature, light, chemicals, irradiation, pH, osmotic pressure, nutrient starvation, toxins, atmospheric pressure, O₂ levels, or temporal controls.

For example where a riboswitch is operably linked to a nucleic acid molecule comprising a coding sequence, in the same genetic construct, a promoter sequence may be provided which regulates transcription of both the riboswitch and the coding sequence. Alternatively, transcription of the riboswitch and the coding sequence may be under the control of separate promoters, which may be either the same or different type. Similarly, in the second aspect of the invention where a first genetic construct comprises a first nucleic acid molecule encoding a riboswitch operably linked to a second nucleic acid molecule encoding a polymerase, the riboswitch and the polymerase may be transcribed under the control of the same promoter. In the second construct provided in the second aspect of the invention, the coding sequence may be under the control of a promoter which is the same or different to either of the promoters of the first construct. In the third aspect of the invention, where a genetic construct comprises two or more riboswitches, each operably linked to a nucleic acid comprising a coding sequence, it is envisaged that one or more riboswitch/coding sequence pair may be under the control of a separate promoter, and/or that two or more pairs of riboswitch/coding sequence may be under the control of a single promoter.

For regulation of eukaryotic sequences, one or more enhancer sequences may also be provided in a genetic constructs of the invention. In the present invention, an enhancer sequence is a DNA sequence which regulates transcription of a coding sequence, in combination with a promoter sequence, and which may be positioned within no fixed distance from the coding sequence, and either 3′ or 5′ thereto. An enhancer sequence is typically between 10 and 300 bp in length. In a genetic construct of the present invention, a promoter sequence may be optionally operably linked to an enhancer sequence. Preferably, two or more promoters may be operably linked to a single enhancer sequence. An enhancer may be derived from any suitable source. Preferred enhancers for use in the present invention include CMV, IGHM, TEF-1, TEF-2, TEF-3 enhancers.

In addition to promoter and/or enhancer sequences, a genetic construct of the invention may also comprise a regulatory element capable of terminating transcription. An example of such an element is a polyA segment in an untranslated region of an mRNA transcript. Other suitable transcription termination sequences are known to the person skilled in the art, and include for example Rho dependent (eg. Graham. NAR, Vol. 32:3093 (2004), Clampi. Microbiol. 152: 2515 (2006). Rho independent (intrinsic) terminators, natural and synthetic, eg. any hairpin loop, any CG rich dyad and specific terminator such as T7(TE) or T1 (rrnB). These elements must be compatible with the remainder of the genetic construct. These elements may not be necessary for the expression or function of the nucleic acid molecules but may serve to improve expression or functioning of the nucleic acid molecule by affecting transcription, stability of the mRNA, or the like. Therefore, such elements may be included in the expression construct to obtain the optimal expression and function of one or more of the nucleic acid molecules.

Additional regulatory sequences include any nucleotide sequence which is capable of influencing transcription or translation of a coding sequence product, for example in terms of initiation, rate, stability, downstream processing, secretion, and mobility. Examples of regulatory sequences include promoters, 5′ and 3′ UTR's, enhancers, transcription factor or protein binding sequences, start sites and termination sequences, ribosome binding sites, recombination sites and polyadenylation sequences.

Ribosome binding sites include optimized Shine Dalgarno/Kozak sequences. Preferred sequences are those comprising the consensus sequences (prokaryote) AGGAGG; (E. coli) AGGAGGU; (Kozak) CCCGCCGCCACCATGGAG.

Thus, in a preferred embodiment of the present invention, a genetic construct of the invention may comprise a transcription termination sequence associated with each transcription unit (i.e. the nucleic acid molecules under the control of any particular promoter and which are transcribed as a single RNA sequence).

### Marker Genes

The genetic construct of the invention may comprise one or more reporter or marker genes. Such genes are preferably included to indicate A) delivery of the genetic construct to the expression system or a host cell; B) expression of the riboswitch transcript; C) conformational change of the RNA sequence comprising the riboswitch; and D) the activity and quantification of activity of the riboswitch.

A. To indicate delivery of the genetic construct to the expression system, a reporter gene may be provided in the genetic construct. The reporter gene may be operably linked to one or more other nucleic acid molecules in the genetic construct, or may be provided within the genetic construct but independent from the other nucleic acid molecules of the construct, and under the control of its own regulatory sequences. Suitable reporter genes for determining delivery of a genetic construct to an expression system include E. Coli lacZ, and green fluorescent protein, luciferase or selectable reporters such as DHFR, thymidine kinase, mycophenolic acid; and resistance to antibiotics such as neomycin, neomycin analogue G418, hydromycin, and purocin, Kanamycin^{R}, Tetracyclin, Zeocyin, ampicillin^{R}, and hygromycin; ancillary regulators such as Laclq, arabinose repressor, Tet repressor, tryptophan repressor, LuxR, and lambda repressor (cl); and Chloramphenicol Acetyl Transferase. Methods of using such reporter genes are well known in the art.

B. To determine expression of a riboswitch (i.e. transcription/translation thereof), a nucleic acid molecule encoding a reporter gene may be provided, operably linked to a nucleic acid molecule encoding a riboswitch. Thus, the expression of the riboswitch can be monitored by observing expression of the reporter gene.

C. The genetic construct may also comprise reporter genes which provide a signal dependent upon a conformational change in the riboswitch, thus indicating activity of the riboswitch. For example, the fluorescent output or wavelength may change dependent upon the structural conformation of the riboswitch to which it is operably linked.

D. A reporter gene may also be provided, operably linked to a riboswitch, which enables monitoring of activity and optionally quantification of riboswitch activity. Thus, preferably, the reporter gene for this purpose will be one whose output (e.g expression levels or activity) correlates to activity of the riboswitch.

Preferably, a reporter gene for use in the present invention as described above produces a detectable signal, being one which can be observed using standard techniques, such as immunoassay, radioimmunoassay, fluorescence detection, luminescence detection, western blot, absorbance, assays for protein activity. Suitable reporter genes include, for example molecules.

Examples of these are known in the art. Preferred reporter genes are those which encode a protein which provides a detectable signal which is easy to observe, for example, luminescence or fluorescence, and which is observed even at low expression levels.

Other reporter molecules such as fluorescent molecules and radioactive isotopes may also be used.

Preferably, a reporter gene will be used which is capable of combining two or more of functions A-D above. One or more suitable reporter genes may be used in a genetic construct of the invention.

Suitable reporter genes include E. Coli lacZ, and green fluorescent protein, luciferase or selectable reporters such as DHFR, thymidine kinase, mycophenolic acid; and resistance to antibiotics such as neomycin, neomycin analogue G418, hydromycin, and purocin, Kanamycin^{R}, Tetracyclin, Zeocyin, ampicillin^{R}, and hygromycin; ancillary regulators such as Laclq, arabinose repressor, Tet repressor, tryptophan repressor, LuxR, and lambda repressor (cl); and Chloramphenicol Acetyl Transferase.

### Ligands

The ligands which activate the riboswitches used in the present invention may be any which are capable of binding to the aptamer domain of the selected riboswitch, and mediating the structural change in the confirmation of the expression platform, which enable the riboswitch to regulate nucleic acid sequence expression. The ligands may be naturally occurring or may be artificial or synthetic. They may be isolated or purified. They may be the native ligand which triggers the riboswitch in nature, or may be a different ligand to the native ligand.

The ligand may serve to activate, inhibit or block a riboswitch. Thus, a ligand may be a non-native ligand of a riboswitch, which serves to inhibit rather than activate the riboswitch. Alternatively, a ligand may be a modified form of a native ligand, or an analogue of a native ligand, which serves to block gene regulation by the riboswitch when bound to the aptamer domain.

Preferred ligands are those which interact with riboswitches including cyclic di-GMP, SAH, pre-Q, Moco and SAM-IV, add-A and purine riboswitches. The invention includes the use of such known ligands which have been modified in relation to their binding to the aptamer domain, for example either enhanced or decreased binding and a resulting effect on expression platform configuration and preferably on coding sequence expression.

Such compounds include, for example, cyclic di-GMP, pGpG, GpG or GpGpG, derivatives of cyclic di-GMP, pGpG, GpG or GpGpG which are capable of activating a cyclic di-GMP responsive riboswitch, S-adenosylhomocysteine, derivatives of S-adenosylhomocysteine that can activate an S-adenosylhomocysteine-responsive riboswitch, preQ1 &/or preQ0, derivatives of preQ1 &/or preQ0 that can activate a pre-Q-responsive riboswitch, Moco, derivatives of Moco that can activate a Moco-responsive riboswitch, SAM, and derivatives of SAM that can activate a SAM responsive riboswitch, and purines or derivatives thereof which can activate a purine riboswitch.

It will be appreciated that the compounds of the invention will represent suitable ligands for use in many aspects of the invention.

For the avoidance of doubt, references to the compounds of the invention in the context of the present disclosure should be taken as encompassing the compounds per se, and also suitable salts or other derivatives thereof. Salts of the compounds of the invention may be of particular utility in contexts where the compound per se has relatively low solubility, or where it is otherwise desired to improve solubility of the compounds.

A suitable salt of the compound of the invention is, for example, an acid-addition salt of the compound formed with an acid such as hydrochloric, hydrobromic, sulfuric, phosphoric, trifluoroacetic, formic, citric or maleic acid. In a particular embodiment, the compound of the invention may be in the form of a hydrochloride salt.

### Methods

It is envisaged that the genetic constructs of the invention and the ligands which are provided to activate one or more of the riboswitches described herein may be used in a variety of gene expression based techniques, several examples of which are provided herein.

The present invention also provides a method of detecting the presence of absence of an analyte, comprising providing in a cell or an expression system a genetic construct of the first aspect of the invention, wherein the coding sequence is a signalling molecule; and detecting the presence of absence of the signalling molecule, wherein presence of the signalling molecule indicates presence of the analyte which is a ligand of the riboswitch of the genetic construct.

### Host Cells and Expression Systems

The invention may be provided as an in vitro expression system or in vivo, within a host cell. Suitable in vitro expression systems include wells, test strip, chip, plate or in vitro cell-like expression systems.

Host cells may be eukaryotic, or prokaryotic. Preferred host cells include yeast, bacteria, plant, animal or mammalian cells. Preferred bacterial cells include, but are not limited to, Escherichia coli, Bacillus Subtilis, Clostridium difficile, Clostridium botulinum, Staphylococcus aureus, lactococcus lactis, pseudomonas fluorescens, bacillus anthracis.

Non-bacterial expression systems include, for example, chloroplasts and cell-free systems.

### Kits

Kits of the invention may additionally comprise instructions for use, and one or more additional components selected from buffers, deoxyribonucleic acids, ribonucleic acids, supports, enzymes, biological strains both engineered and non-engineered including, bacterial strains, cell lines, plasmids, components for transfection and transfection, inducer molecules.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

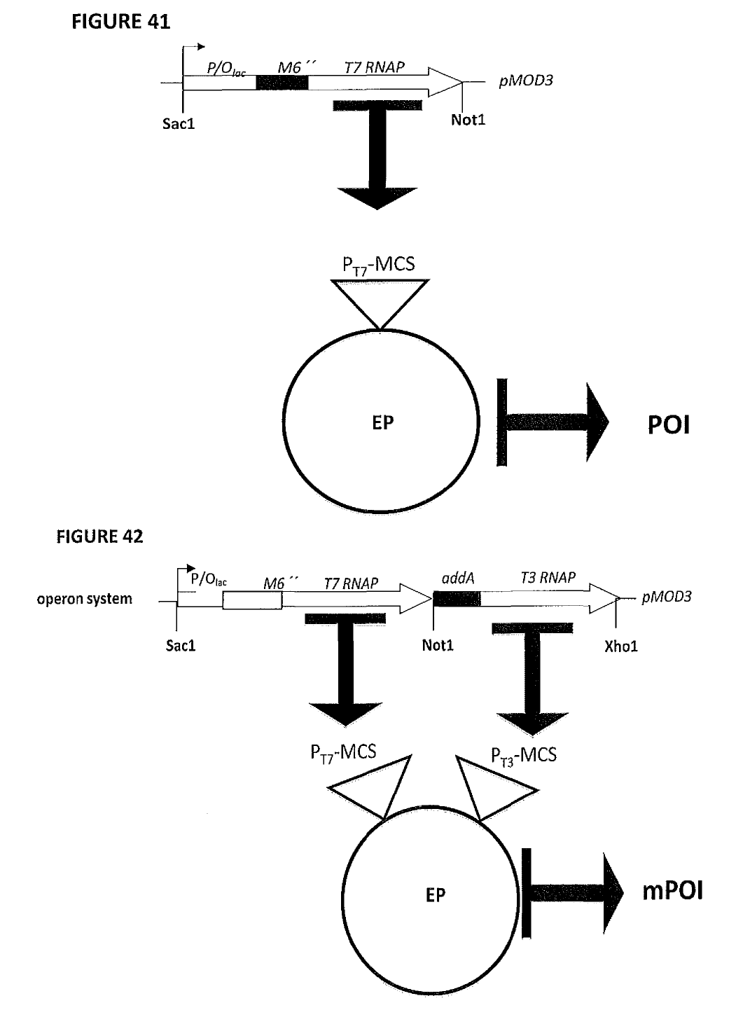

Here we show how two mutually orthogonal riboswitches can be independently controlled by varying concentrations of two distinct synthetic inducers, allowing dynamic expression landscapes to be accessed and optimal co-expression stoichiometries to be established. In the first instance a dual promoter system was constructed, in the pBKS vector with the orthogonal M6 riboswitch selected previously (Dixon et al Proc Natl Acad Sci USA 2010, 107, 2830] or Dixon et al Angew. Chem. 2012, 51, 3620-3624. controlling translation of the red fluorescent protein DsRed [Bevis, Nat. Biotech. 20:83 (2002)] in response to the ligand ammeline (Am). The bacteriophage lambda tR1 transcription terminator [Graham. NAR, Vol. 32:3093 (2004] was inserted downstream of DsRed gene, followed by a second lac promoter and the 2-aminopurine (2AP)-responsive parental add A-riboswitch [Dixon et al Proc Natl Acad Sci USA 2010, 107, 2830 & Mandal, Nat Struct Mol Biol 11: 29 (2004)] controlling translation of the green fluorescent protein eGFP (FIG. 34A). Escherichia coli was transformed with this dual promoter construct and expression of DsRed and eGFP genes, was then determined by fluorescence as function of inducer concentrations.

To explore the capacity of the dual promoter system for controlling the differential expression of DsRed and eGFP, multi-dosage matrix expression analysis was performed where induction factors (IF), the ratio of the maximum ligand-induced protein expression over the basal protein expression level, are determined for a full range of inducer concentrations (FIGS. 34A & B). Following, Co-administration of Am and 2-AP, it can be seen that induction of DsRed expression with increasing concentrations of Am reaches a maximum induction factor of 9.3, whilst the simultaneous increase in 2AP has essentially no effect upon the DsRed induction (FIG. 34B). Similarly, dose-dependent regulation of eGFP is observed upon increasing addition of 2AP, reaching an induction factor of 11.3, with changes in Am concentration having no effect upon the induction of eGFP expression (FIG. 34C). Whilst the orthogonal selectivity of M6 and add-A has been previously determined in vitro [Dixon et al Proc Natl Acad Sci USA 2010, 107, 2830], these results show for the first time the two riboswitches, despite being similar in sequence and structure, possess excellent in vivo orthogonality and can be used to independently control co-expression of the two genes simultaneously.

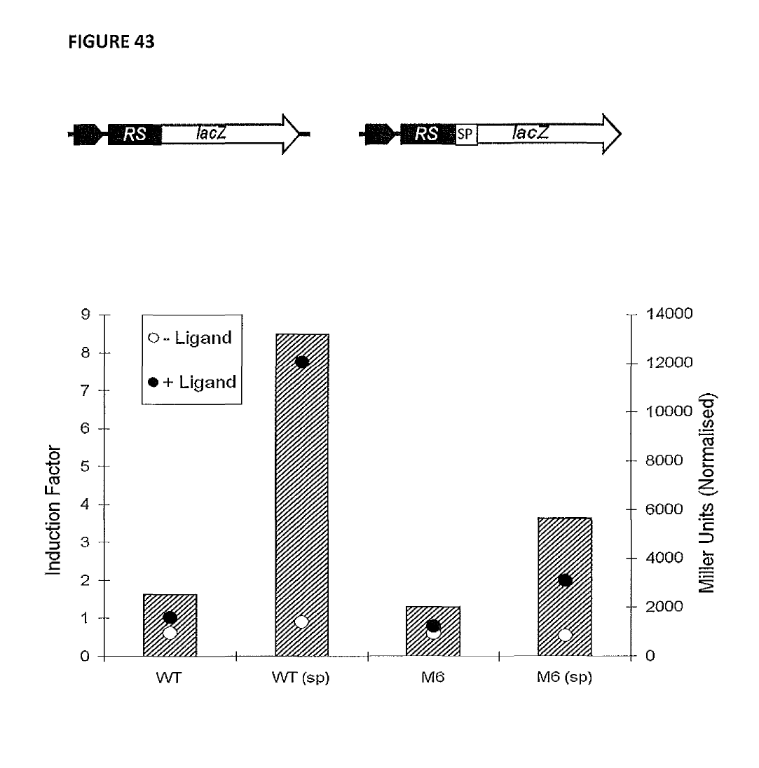

In a second series of experiments, a synthetic operon was designed with one promoter driving transcription of a single bicistronic mRNA comprising the M6 riboswitch regulating DsRed, and an intergenic add-A riboswitch controlling a downstream eGFP gene (FIG. 35A). Matrix analysis of the induction factors demonstrates differential control of DsRed can be achieved. Moreover, the maximum DsRed induction factor was 21.2, which is also more than 2-fold higher than the dual promoter system (FIG. 35B). The organisation of M6-DsRed in an operon, permits differential control, but also gives rise to significant amplification of the dynamic range of gene expression (IF), compared with the dual promoter system. Analysis of eGFP expression also showed very interesting and unexpected results. In this case, eGFP output was clearly dependent on both ligands. A synergistic effect of the two ligands is observed in the induction factor matrix analysis, across a full range of Am and 2AP concentrations (FIG. 35C), with the expression landscape reaching a maximum induction factor of 24.1 in the presence of the highest concentrations of both inducers. Our earlier in vitro studies, clearly show that there is no binding interaction with Am and add A or between 2AP and M6. Similarly no cross-talk is observed with the dual promoter system described above. This suggests that eGFP expression within operon system is not only regulated by the intergenic add A-riboswitch but also by the upstream M6 riboswitch.

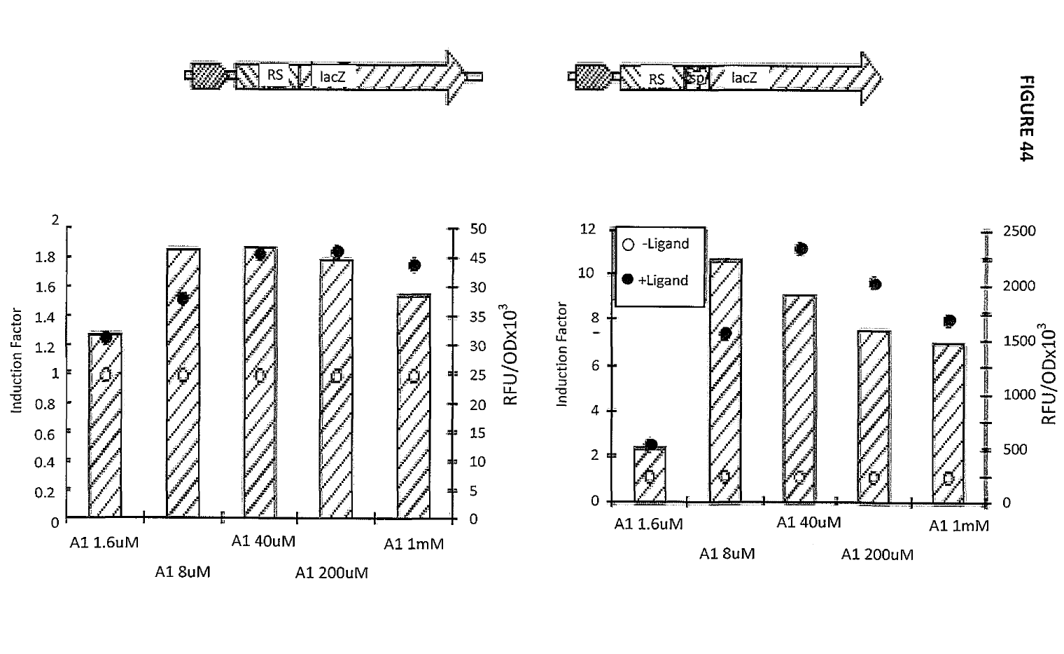

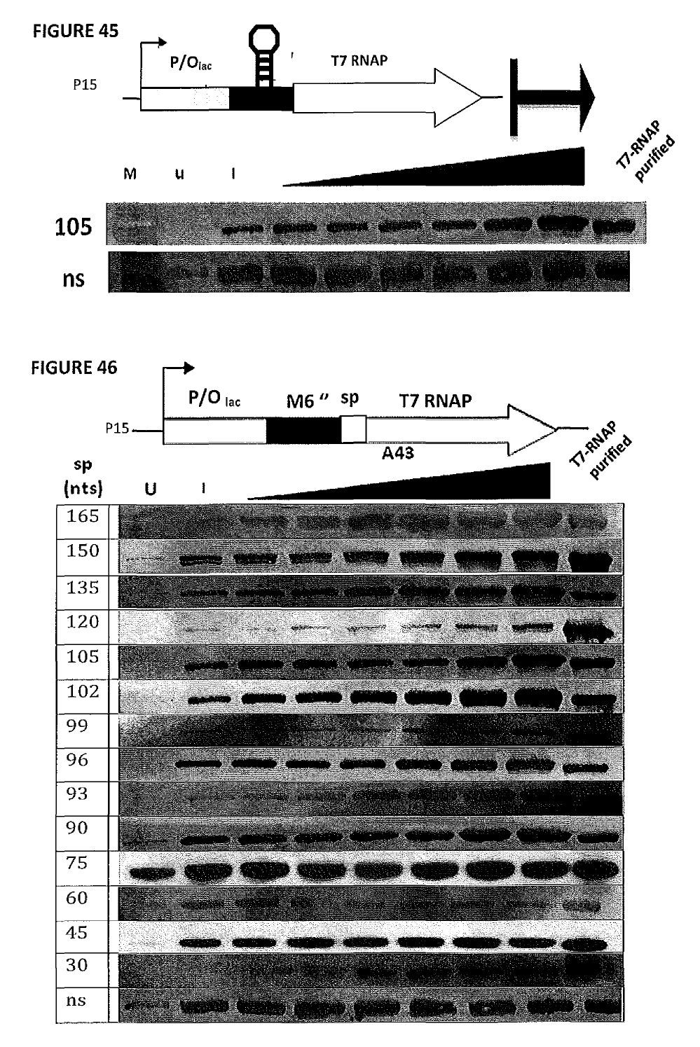

A number of reasons could possibly account for this. Firstly, ribosomal read-through could result in a more efficient initiation of translation at the eGFP RBS. Secondly, riboswitches that control gene expression through changing the accessibility of the RBS (like addA), may also rely on Rho-dependent termination of transcription [Roth, Annu. Rev. Biochem. 78:305 (2009) & Peters Proc. Natl. Acad. Sci. USA 106:15406 (2009)]. In the case of the operon, when both riboswitches are activated by Am and 2AP, ribosomes will be recruited at both RBSs and will occupy the full length of the transcript, blocking Rho binding [Epshtein, Nature 463:245 (2010)]. Consequently the levels of full-length mRNA transcripts in the cell will be higher, than if either or both ligands are absent. Finally protection of the full-length transcript from nuclease degradation by actively translating ribosomes [Deana, Genes Dev. 19:2526 (2005)], recruited at both RBSs, would also serve to increase mRNA stability in the presence of both inducers. Whichever mechanism is prevailing it is clear that the presence of two riboswitches within an operon system affords greater digital control of gene expression, which is also demonstrated by dose dependent analysis (FIG. 36-37). Titration of the M6 selective inducer Am (0→250 μM) against the dual promoter in either the absence or presence 2AP (0 or 250 μM) gives dose-dependent protein expression curves for DsRed that are very similar, with maximum normalized red fluorescence (fu/OD₆₂₀) of 1.1×10⁵ and 1.0×10⁵ respectively (FIG. 36A). Dose-dependent control of eGFP expression is also observed upon addition of the add A-selective inducer 2AP (FIG. 36B) and eGFP expression is also clearly unaffected by the presence of the M6 ligand (Am), with maximum eGFP levels of ca. 55×10⁶ fu/OD₆₂₀. In the case of the operon construct titration of Am (0→250 μM) in the presence or absence of 2AP (0 or 250 μM) produces essentially identical dose-response curves for DsRed expression, where a maximum output both of 2.9×10⁶ fu/OD₆₂₀ is observed (FIG. 37A). However for the eGFP gene the synergistic effect observed above is also seen, where upon titration of 2AP alone produces maximum output of 14.8×10⁶ fu/OD₆₂₀, and in the presence Am (250 μM) an output 25.9×10⁶ fu/OD₆₂₀ (FIG. 37B). Indicating that both inducers serve to amplify expression of the downstream eGFP gene, within the operon.

In nature, bacterial riboswitches are typically found in the 5′-UTR sequence of the mRNA, controlling expression of single genes and operons, to date no natural riboswitches have been found to function within intergenic regions (as in the synthetic operon FIG. 34A). There are however several examples in nature where tandem riboswitches, within 5′-UTR sequences, have been shown to regulate a single gene output [Sudarsan, Science 314: 300 (2006) & Welz, RNA 13:573 (2007)], providing similar ‘digital’ control as the synthetic operon system (FIG. 34A).

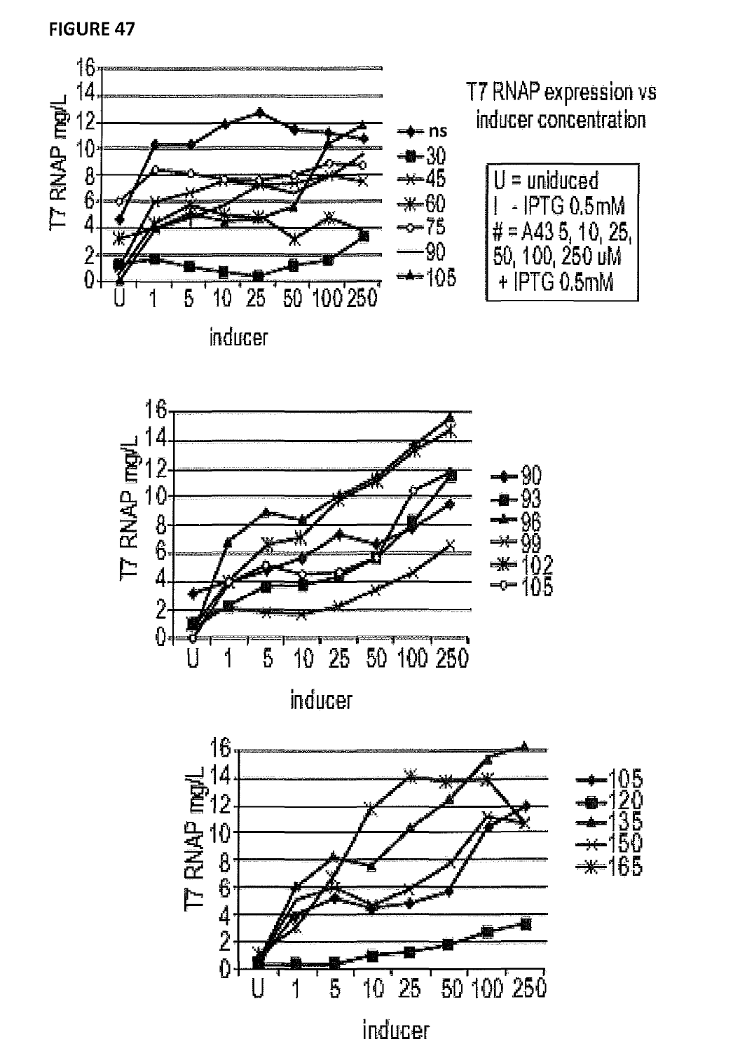

Fluorescent Microscopy was used to ascertain whether the observed independent expression of the two reporter genes occurs within individual bacterial cells. The microscopy images of E. coli cells transformed with the dual promoter or operon construct (FIG. 38A-B) show that the individual cells emitting both green and red fluorescence upon co-administration of both Am and 2AP (250 μM) and that co-localisation of fluorescence is clearly seen in the overlay frame. This indicates co-expression of DsRed and eGFP occurs within individual E. coli cells, for both systems, and that the observed expression profiles are not due to differential expression of sub-populations of cells, which would be problematic for many of the applications described above.

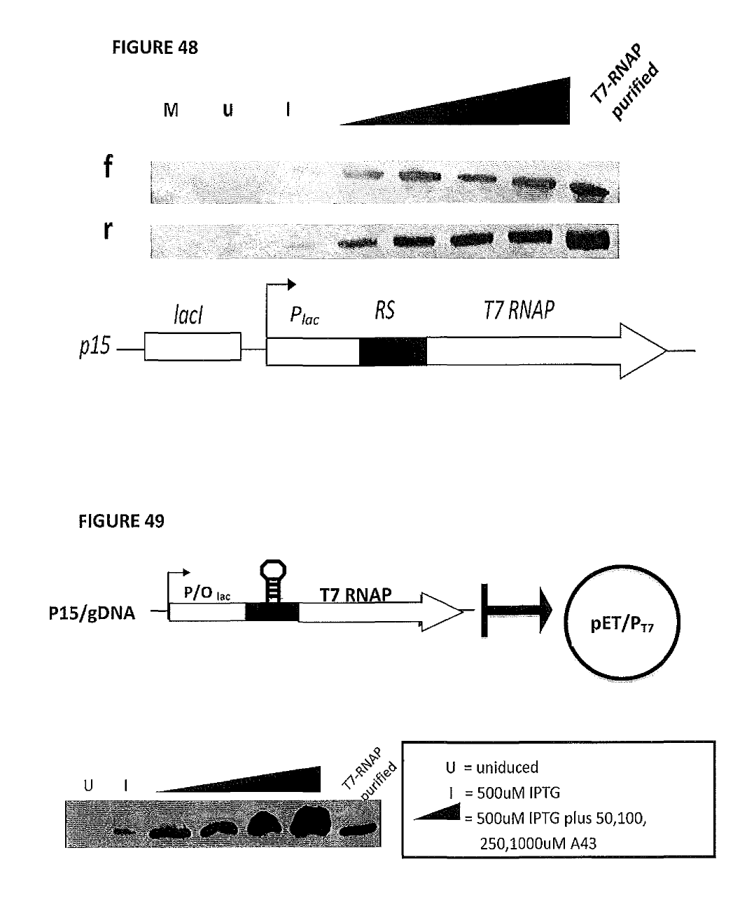

To further demonstrate how mutually orthogonal riboswitches, can regulate the stochiometry of two gene products, the normalised gene expression outputs for the both the dual promoter and operon systems were used to generate 2D contour maps of the expression landscapes (FIGS. 39 & 40). From these contour maps it is possible to see how appropriate inducer concentrations can easily be selected so that the desired defined stoichiometry of the two gene products can be obtained.

In summary, mutually orthogonal riboswitches can be used control the differential expression of multiple genes, in the same cell, simultaneously. The Dual Promoter system permits simultaneous and independent control over two genes with no “cross-talk” between the genetic control elements. Using 2D-contour plots of the resulting dynamic expression landscapes, it is possible to select appropriate concentration of small molecule inducers required to obtain a specific stoichiometry of gene products. Moreover when two mutually orthogonal riboswitches are deployed in the 5′-UTR sequence and in the intergenic region of a bicistronic mRNA, significant amplification of expression of the downstream gene is observed, allowing more digital control over gene expression. A synthetic Operon system, in which riboswitches are deployed in both the 5′-UTR and intergenic region of a bicistronic mRNA, was shown to afford a greater level of control over the dynamic range of inducer response, wherein the dynamic range of control that is afforded by the downstream intergenic riboswitch is controlled by the upstream 5′-UTR riboswitch.

Given that there are very few reliable methods currently available to accurately balance expression of multiple protein outputs [Pfleger 2006], the method described here could have wide-ranging biotechnological applications and new gene expression tools.

### Screening for New Riboswitch Ligands

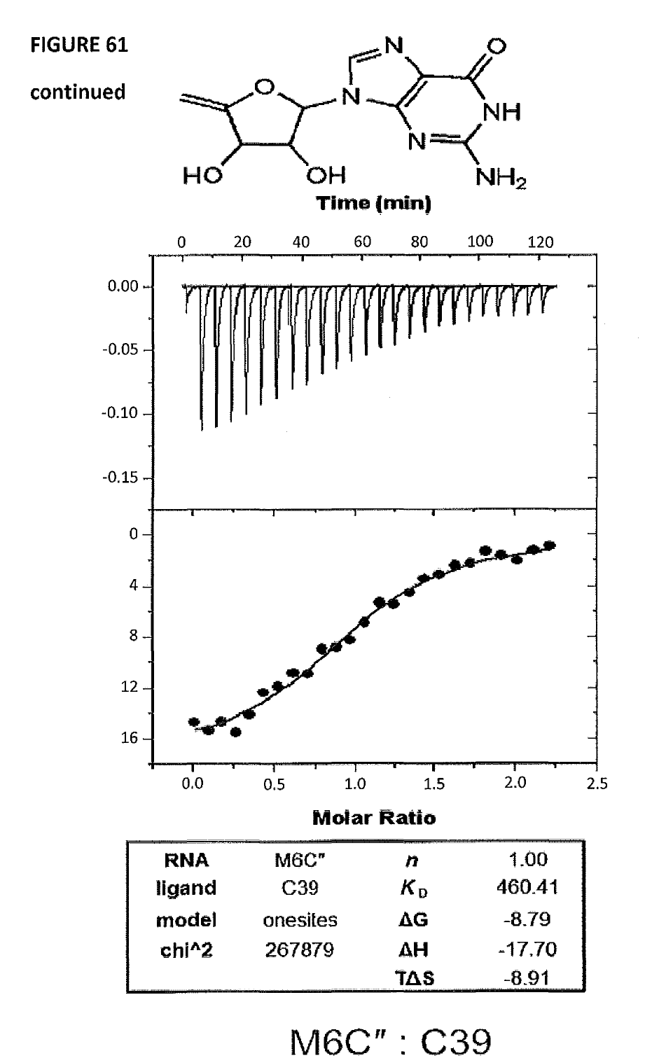

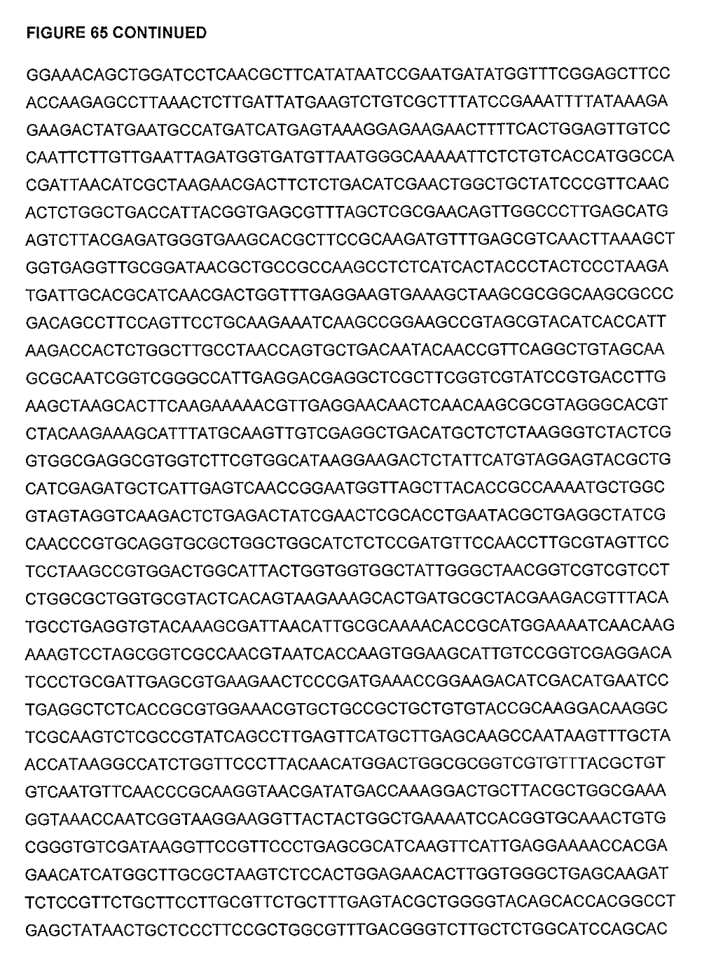

Re-engineering the adenine responsive add A-riboswitch (addA) from Vibrio vulnificus (FIGS. 27A & B) [Dixon et al Proc Natl Acad Sci USA 2010, 107, 2830]. By stabilising the P2 stem of the mutants, it was possible to engineer two improved riboswitches M6″ and M6C″, which produced higher eGFP expression levels than the wild-type. Crucially both mutant riboswitches are completely orthogonal to the parental riboswitch as neither responds to adenine or other purines present the cell, whilst addA does not respond to the synthetic ligands 2 and 3 (FIGS. 27C and F). Isothermal titration calorimetry (ITC) experiments confirmed that the M6″ and M6C″ aptamer domains have no affinity for adenine, but bind respectively to ligands 2 and 3 with K_{d} values in the 1.0-1.2 μmol range. Finally, a 1.7 Å crystal structure of the M6C″ aptamer domain in complex with azacytosine 3 (3LA5.pdb) was obtained, which shows how the riboswitch can adapt its structure to accommodate alternative synthetic ligands (FIG. 27D-F). This structural information has been used to guide screening of a number of alternative heterocyclic compounds, from which we discovered a superior M6″ ligand pyrimido[4,5-d]pyrimidine-2,4-diamine (A43) that affords a 12-fold induction of gene expression in E. coli, which is higher than the parental riboswitch. This ligand has been shown to bind to the M6″ riboswitch by isothermal titration calorimetry with a K_{d}=189 nM (FIG. 59) Used in combination with an inducible promoter such as the lac promoter/operator this induction can be extended >400-fold (FIG. 53). It was also shown how the orthogonally selective riboswitches can be used for the differential and simultaneous control of two genes in the same E. coli cell.

Controlled expression of toxic and insoluble proteins using orthogonal riboswitches.

The orthogonal riboswitches generated to date exhibit very low basal levels of expression, compared with the lac-IPTG system. The aim was to develop protein expression plasmids for E. coli, employing orthogonally-inducible single and tandem riboswitches, which are essentially silent in the absence of the inducer ligand. These expression plasmids can then be used to demonstrate the controlled production of selected toxic proteins, which have proven difficult to produce in E. coli by conventional means. Another major problem is the insolubility of some recombinant proteins when overproduced in bacteria. This can be alleviated, to some extent, by using an inducible promoter to reduce the rate of protein synthesis and promote proper folding. However, the all-or-none behaviour of the lac-IPTG system, means that even at low IPTG concentrations whilst some cells remain un-induced, others become fully induced and consequently rates of protein synthesis cannot be controlled at the cellular level. The orthogonal riboswitches developed will allow much more stringent and quantitative (dose-dependent) control of both the transcriptional and translational levels. The expression plasmids, carefully optimising concentrations of riboswitch ligands, were tested for production of proteins that are difficult to produce in soluble form in E. coli. In parallel, E. coli expression strains were engineered with a copy of the T7 viral RNA polymerase gene integrated into the chromosome under the control of an orthogonal riboswitch. Such E. coli strains would therefore be compatible with commercial expression plasmids such at the pET vectors.

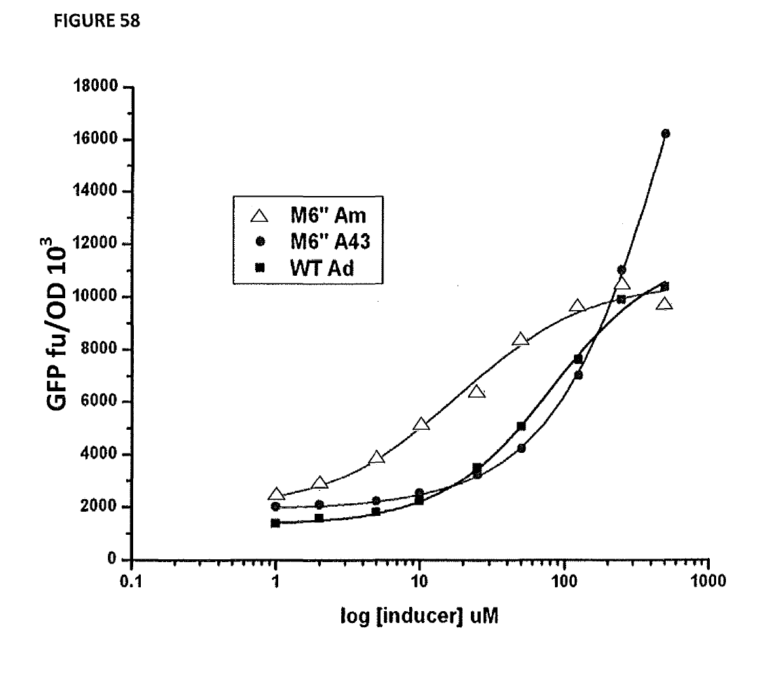

FIG. 48 illustrates induction of T7 RNA polymerase in response to increasing concentrations of a riboswitch ligand. As shown in the schematic representation of the construct the T7 RNA polymerase is under the control of a riboswitch downstream from a lac promoter/operator which is itself downstream from a lac I repressor. Two versions of this construct were created in which the lac I repressor was in either the same direction as the T7 RNA polymerase open reading frame (designated “f” for forward) or was in the opposite direction (designated “r” for reverse). As can be seen from the blot data presented, increasing concentrations of the riboswitch ligand A43 (provided at concentrations of 50 μM, 100 μM, 250 μM, or 1000 μM) gave rise to increased induction of T7RNA polymerase in a dose-dependent manner, irrespective of the direction of the lac I repressor.

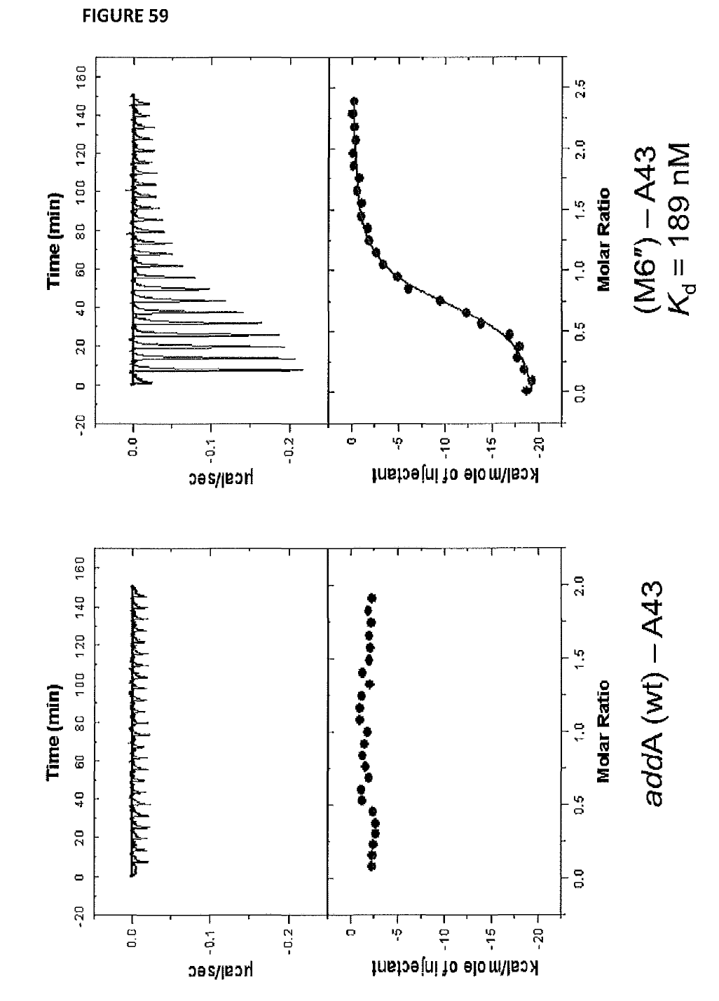

FIG. 49 illustrates a means by which modular gene expression may be achieved using a genetic construct in accordance with the present invention. T7 RNA polymerase (T7 RNAP) induction is controlled by both the lac promoter/operator (P/O lac), and the 5′UTR riboswitch (top). The blot data set out in this Figure illustrated that the gene construct allows dose-dependent control of T7 RNAP in response to the presence of IPTG and increasing concentrations of the riboswitch ligand A43. The riboswitch ligand controls induction of T7 RNA polymerase, and this, in turn, controls the expression of any gene of a further gene of interest cloned within a vector containing the T7 promoter (such as the PT7/pET vectors pictured on the right hand side of the Figure). Thus the riboswitch ligand indirectly controls expression of a gene of interest in this decoupled system.

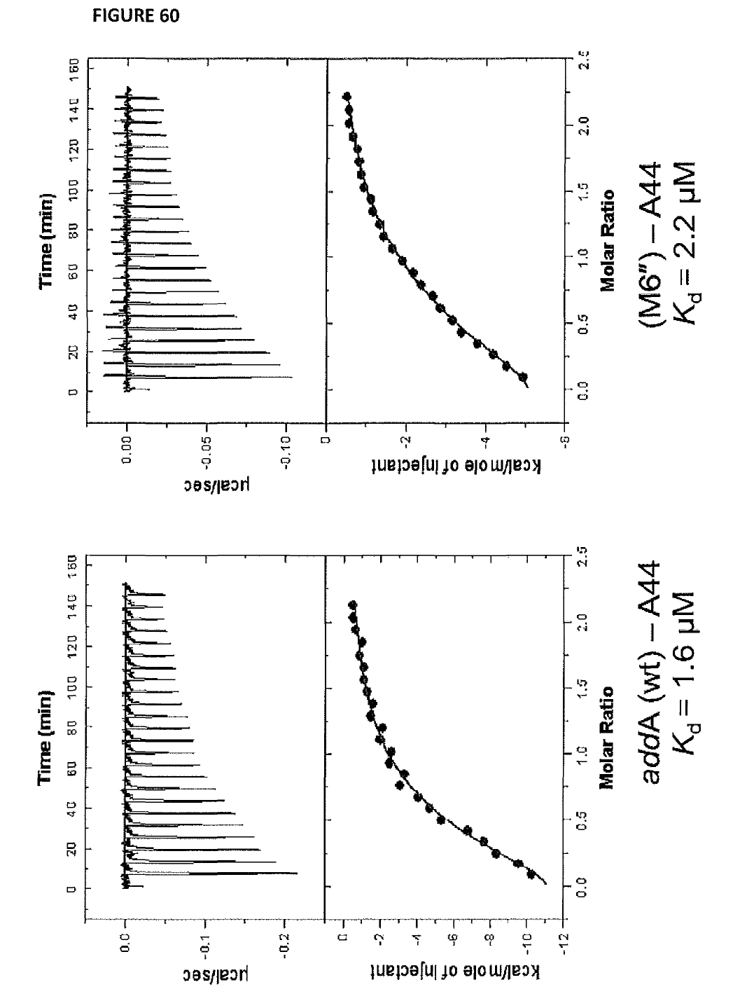

Modular control of this sort is further demonstrated in FIG. 50. Here a gene construct of the invention incorporates T7 RNA polymerase under the control of a riboswitch, with induction of the T7 RNA polymerase then controlling expression of a gene of interest coupled to a T7 promoter.

The blot data shows results of incorporation of these constructs in BL21(DE3) cells (lanes 2-3) and Top10F′ cells (lanes 4-10) both strains having been transformed with pET45_MppK expression vector. Addition of IPTG to BL21(DE3) leads to the expected expression of the gene of interest, in this case the His-tagged 50 kDa protein MppK. Upon addition of the addition of IPTG and the riboswitch inducer, the protein of interest is expressed in a dose-dependent manner.

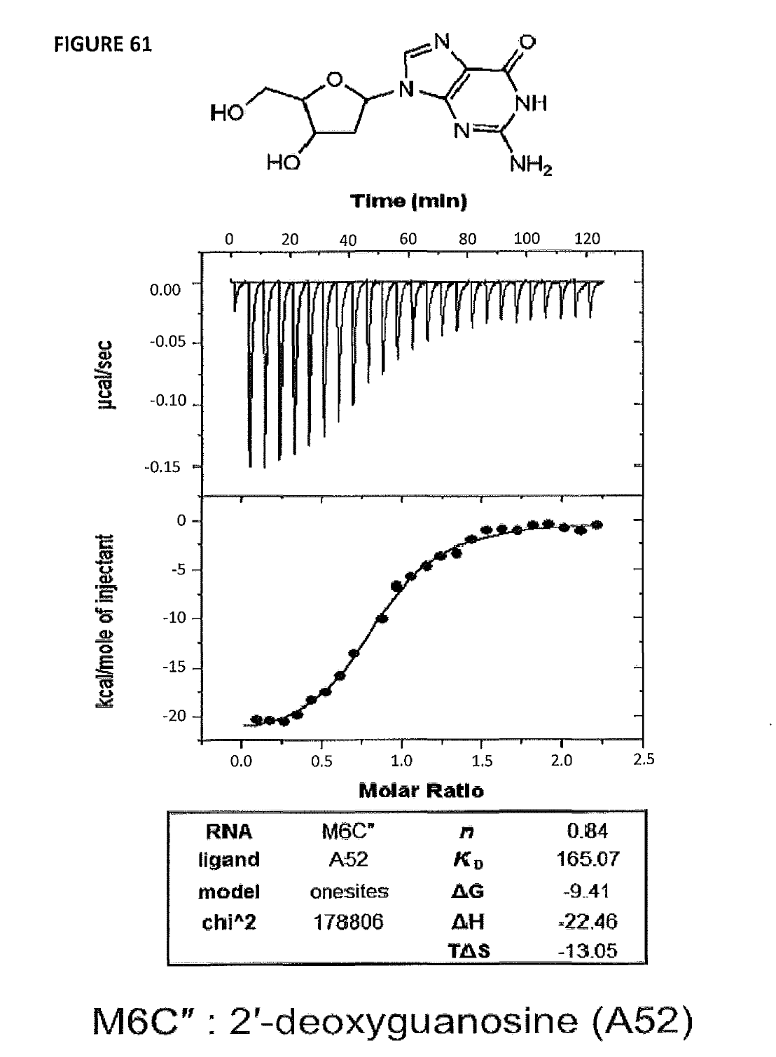

FIG. 51 shows data generated from a similar model in which T7 RNA polymerase under the control of a riboswitch and lac promoter/operator is used to control expression of green fluorescent protein in a pET-GFP vector. These expression data (shown in the right hand part of the graph) are compared with expression data from pET-GFP in BL21(DE3) cells (in the left hand part of the graph).

It can be seen that GFP expression in the BL21(DE3) cells is “leaky”, whilst GFP expression in the BL21 p15_LOST1 cells comprising systems in accordance with the second aspect of the invention exhibit far tighter control of GFP expression. In these cells comprising a system of the invention IPTG provides transcriptional control of GFP expression, while the riboswitch ligand A43 provides translational control of GFP expression. Upon addition of both IPTG and A43 dose-dependent control of the gene of interest (GFP) from pET-GFP is observed.

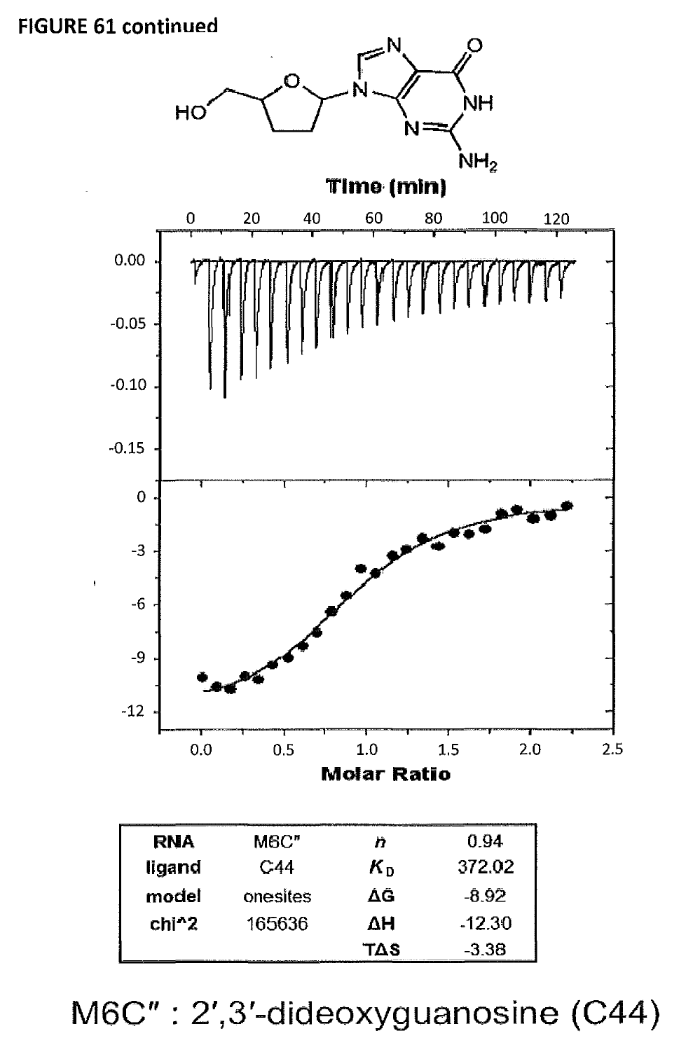

FIGS. 52 and 53 illustrate the increased dynamic regulation of expression of a gene of interest (here exemplified by green fluorescent protein) that can be achieved using gene constructs in accordance with the present invention, as compared to comparator (control) constructs. In the control constructs GFP expression is controlled directly by a lac promoter/operator alone. In contrast, in the gene constructs of the invention GFP expression is controlled by both a lac promoter/operator, and a riboswitch. In this arrangement the lac promoter provides transcriptional control of expression, and the riboswitch provides translational control. FIGS. 52 and 53 respectively show data generated by gene constructs of the invention comprising first and second generation riboswitches (referred to respectively as “1^{st} Generation” and “2^{nd} Generation” constructs) when present on high copy plasmids in E. coli Top10F′. In this example, the first generation riboswitch comprises AddA (the ligand for which is adenine), and the second generation riboswitch comprises M6″ (the ligand for which is A43). These two riboswitches may generally represent preferred examples to be used when it is desired to utilise two orthogonal riboswitches in an embodiment of the various aspects of the invention.

Turning first to FIG. 52, this shows results comparing GFP expression by cells comprising a control construct and by cells comprising the 1^{st} Generation (addA-eGFP) construct of the invention. GFP induction factors (left axis indicated by the green bars) and absolute normalized GFP fluorescence signal (right axis) in the absence of inducer (open shape) and in the presence of inducer (closed shape).

The induction factor represents the degree to which gene expression is controlled, and is determined from the ratio of the maximum induced protein expression over the basal protein expression level. In the present example, the control lac P/O construct (results for which are shown in column 1) affords GFP expression of ca. ˜100×1O⁶ fu/OD in the presence of 1 mM IPTG, and a basal expression of ca. ˜25×1O⁶ fu/OD without IPTG. Hence the induction factor of this control construct is 4. The riboswitch containing genetic construct of the invention (results for which are set out in columns 2-6) achieves approximately the same maximum GFP expression of around ˜95×1O⁶ fu/OD, in the presence of the riboswitch inducer A1. However, the basal level of expression for this 1^{st} Generation genetic construct of the invention is ˜4×1O⁶ fu/OD, thus the induction factor achieved by this construct is approximately ˜22. These results demonstrate the tight control of the basal level of expression, and an increased dynamic window of expressional control, that may be achieved by the genetic constructs in accordance with the invention as the riboswitch inducer concentration is increased from 10 uM-1 mM.

FIG. 53 illustrates expression control of transcription regulation (using a comparator genetic construct) as compared to dual transcription-translation regulation with a genetic construct in accordance with the invention utilising a second-generation riboswitch and inducer. As before, the comparator genetic construct comprises lac promoter/operator directly controlling the expression of GFP. This construct is shown schematically in the top left of FIG. 53, and results obtained are shown in column 1 of the graph at the bottom of this Figure.

In contrast, the genetic construct of the invention comprises a lac promoter/operator upstream of a 2^{nd} generation riboswitch M6″-eGFP the lac promoter/operator and riboswitch respectively providing direct control of the expression of GFP via transcriptional and translational control. This genetic construct of the invention is shown schematically at the top right of FIG. 53 (where the riboswitch is identified as “RS”), and the results achieved using this genetic construct are shown in columns 2-5 of the graph at the bottom of this Figure, concentrations of riboswitch inducer A43 are shown.

Both the comparator construct and the genetic construct of the invention were provided on high copy plasmids in Top 10F′. Results shown in the graph illustrate GFP induction factors (left axis indicated by the green bars) and absolute normalized eGFP fluorescence signal (right axis) in the absence of inducer (open shape) and in the presence of inducer (closed shape).

The results achieved for the comparator construct, in which GFP expression is controlled solely by the lac promoter/operator, are shown for comparison in column 1, and are the same as referred to in the description of FIG. 52. The results achieved by the genetic construct of the invention incorporating the second generation riboswitch differ from both those obtained using the comparator construct, and those obtained using the genetic construct of the invention comprising a first generation riboswitch. In these results, shown in columns 2-5, the maximum GFP expression observed is ˜120×1O⁶ fu/OD, as for the first generation construct. However, the basal level is ˜0.25×1O⁶ fu/OD, affording an induction factor of ˜450. This demonstrates much tighter control of the basal expression and a large dynamic ‘window’ of expressional control as the riboswitch inducer (A43) concentration is increased 10 uM-1 mM.

When considering the lac P/O control alone (column 1) only 4-fold control is observed +/−IPTG, in comparison with the riboswitch containing construct +/−IPTG (column 2) 40-fold control is observed. This demonstrates that there is a ‘regulatory synergy’ when transcriptional and translational regulatory elements are coupled together, affording tight basal control with addition of precise ‘tuneable’ control over a wide dynamic gene expression range upon addition of the riboswitch inducer.

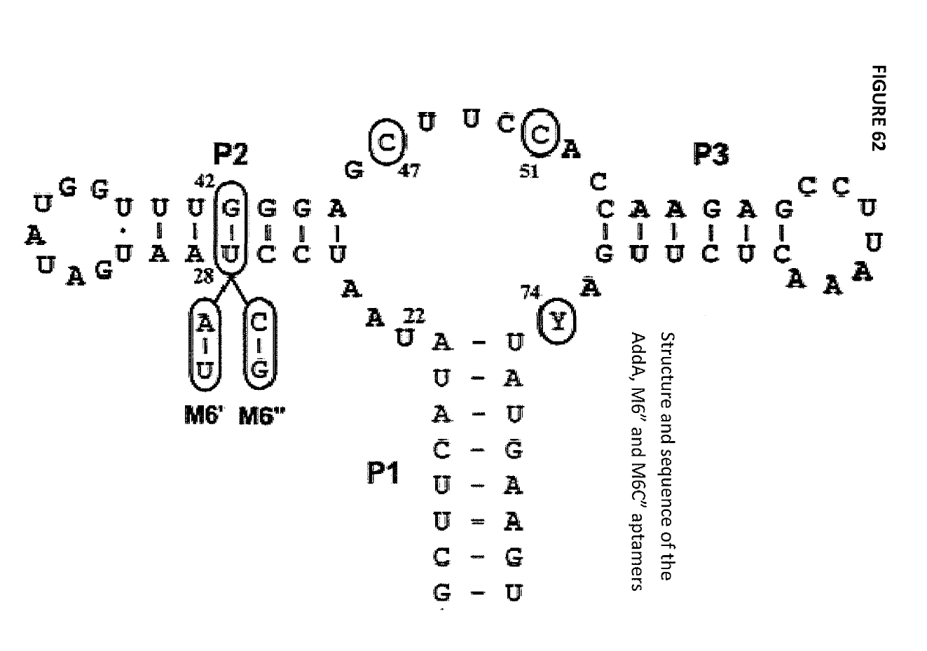

FIG. 54, illustrates cell population control analyzed FACS of cells in which the genetic construct of the invention incorporating a 2^{nd} generation riboswitch controls the expression of GFP. Fluorescence intensity (RFU) is shown on the x-axis, while cell counts with specific RFU are shown on the y-axis. The order of the peaks from left to right correspond to the order shown in the legend, in which “Lac O” refers to treatment with 0.5 mM IPTG, and “1 uM“−”1 mM” refers to concentration of the riboswitch inducer A43 in addition to 0.5 mM IPTG.

The graph illustrates that RFU intensity, indicative of GFP expression, increases along with inducer concentration. Furthermore, the uniform distribution achieved demonstrates that all the cells within the population are expressing GFP at approximately the ‘same’ level. This observation allows the skilled person to match the expression rate of the gene of interest to one or more of a number of different relevant cell characteristics; to synthetic capacity of the cell, to chaperone capacity, and potentially to secretion capacity. Finally, as all of the cells at any given dose of riboswitch inducer are uniform there should also be no difference between protein homogeneity across the cellular population.

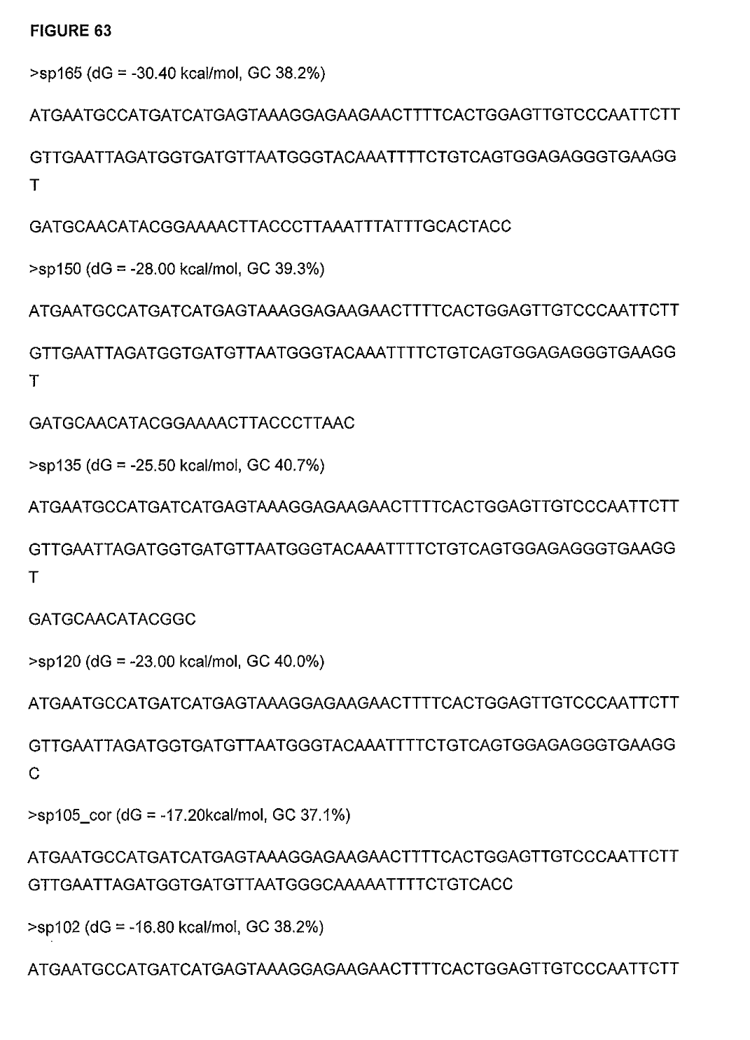

FIG. 55 illustrates use of genetic constructs comprising the riboswitch PreQ₁ to establish conditional mutants for MreB that are of use in target validation and drug screening.

Screening new compounds for antibacterial activity can be a challenging and laborious, and can lead to false positives due to the non-optimised physiochemical properties of compounds in the screening libraries. To aid this screening process methods have been implemented to increase the sensitivity of bacteria to compounds and to help increase the screening hit rate. Notably, where the antisense sequence for a drug target of interest has been placed under the control of an inducible promoter [Wang et al Nature 2006, 441, 358].

As described herein, the inventors have developed a novel method where an antibiotic target gene of interest has been put under control of a mutant PreQ-C17U riboswitch, using in combination a knock-out strain of that gene of interest This conditional mutant demonstrates a wild-type phenotype with respect to growth rate and morphology, but ideally results in a conditional ‘sensitive’ phenotype suitable for drug screening only upon addition of the PreQ-C17U riboswitch ligand D9.

The bacterial actin, MreB, forms helical cables underneath the cytoplasmic membrane and can interact with the enzymes for peptidoglycan biosynthesis [Errington et al Trends Cell Biol. 2003 13, 577-83]. It plays a key role in elongation of the cell wall in most rod-shaped bacteria. Accordingly MreB is a promising target for broad-spectrum antibiotics.

The inventors have developed a gene expression tool, the preQ₁ riboswitch, which can be used to control different level of MreB expression in response to variation of the concentration of the D9 ligand. In keeping with the inventors' expectations, this tool has utility in drug screening, as demonstrated by the present example.

B. subtilis 3725-ΔMreB, as employed in this study, is a magnesium-dependent null mutant which hardly grows in media without magnesium. A construct preQ₁C17U-mreB (in which MreB expression is controlled by the C17U mutant form of the preQ₁ riboswitch described above) was set up within amyE gene fragment as a linear DNA and integrated into amyE locus of B. subtilis 3725-ΔMreB, producing a conditional mutant B. subtilis 3725C. This resulting strain can grow well in media without magnesium, presenting a growth rate of 0.27 (OD/min) which is similar to that of the 3725 wt strain grows in the medium with magnesium (0.29 OD/min). This demonstrates that the preQ₁C17U-mreB construct overexpresses MreB and complements the phenotype of B. subtilis 3725. Furthermore, treatment of D9 (1.25 mM) can repress the growth rate of the 3725C strain to 0.15 OD/min, indiacting that D9 represses the overexpression of MreB, and thus provides a conditional mutant with potential drug screening applications.

In addition to applications of this method for use in antibacterial screening programme, this method also has application for pharmaceutical target validation for ‘hit’ compounds. Where the gene product believed to be target for a hit compound can be placed under a repressible mutant riboswitch construct as above and used to compare phenotype and confirm the hit's cellular target.

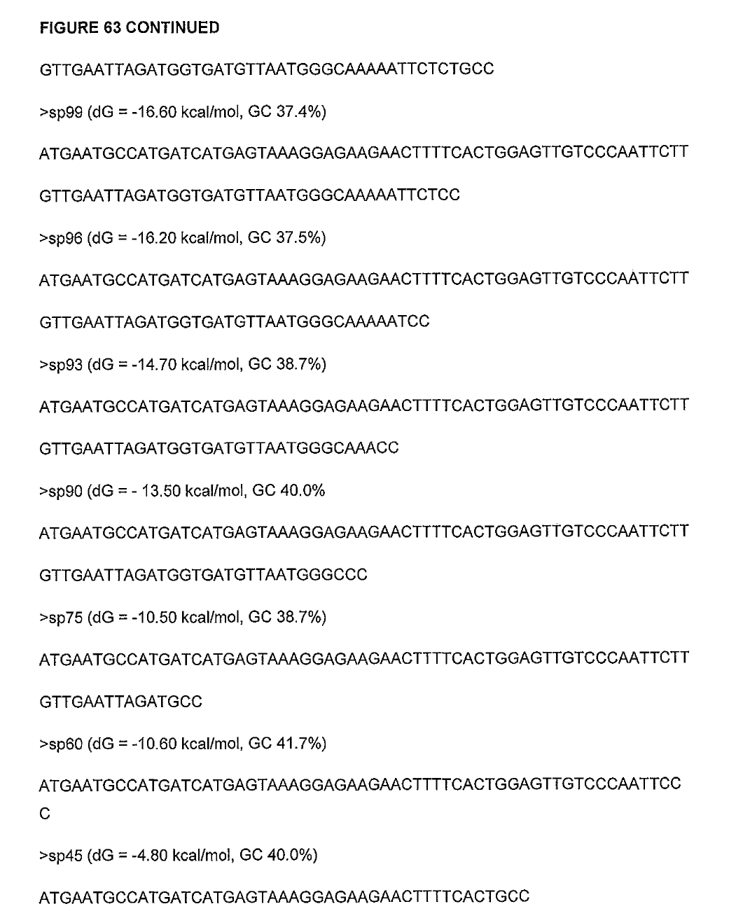

FIG. 56 shows the addA riboswitch connected to the T3 RNA polymerase optimally connected by the spacer detailed in this invention. With addition of IPTG (0.5 mM) and upon increasing concentration of the riboswitch inducer A1 (0.8, 4, 20, 100, 250, 500 uM) dose-dependent control of the polymerase is observed, this is visualised by anti-His6 western blot.

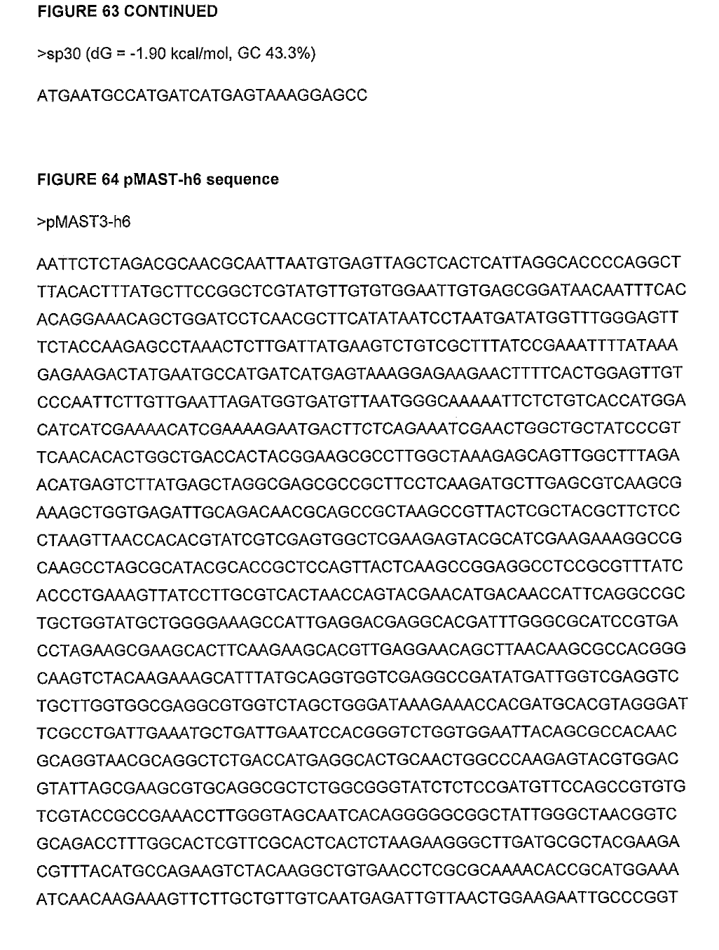

FIG. 57 Dual Promoter construct transcriptionally regulated by a lac promoter/operator, where the M6″ riboswitch—A43 ligand combination control the expression of the spT7 RNA polymerase, separated by at tR1 transcriptional terminator, a second lac promoter/operator and the addA riboswitch—A1 ligand combination control the expression of the T3 RNA polymerase (pTTv9).

Expression induced with A43 at 2, 10, 50, 250 uM in the absence and presence (+) of A1 at 250 uM, and A1 addition at 2, 10, 50, 250 uM in the absence and presence (+) of A43 at 250 uM (b). Addition of A43 (2→250 uM) permits dose dependent regulation of the T7RNAP, in the absence and presence (+) of A1 (250 uM) visualized by western blot with aT7 (c). Addition of A1 (2→250 uM) permits dose-dependent regulation of the T3RNAP, in the absence and presence (+) of A43 (250 uM) visualized by western blot with αHis (d).

By combining mutually-orthogonal riboswitches, this multicomponent Dual Promoter co-expression system has been constructed, which can be used in combination with distinct small-molecule inducer compounds to control the differential expression of multiple genes within the same cell. The Dual Promoter system containing two different viral RNA polymerases (T7 and T3) permits simultaneous and independent control over two genes with no “cross-talk” between the genetic control elements. As the two polymerase are additionally selective for their respective promoter sequences (T7: TAATACGACTCACTATAGG) (T3: AATTAACCCTCACTAAAGG) [Morris et al, Gene, 1986, 41, 193-200], using an expression plasmid such as a modified pET vector containing with two genes of interest under control of the T7 and T3 promoter allows the multicomponent-decoupled system to control the genes of interest as is shown for the single component systems (FIG. 50, 51)

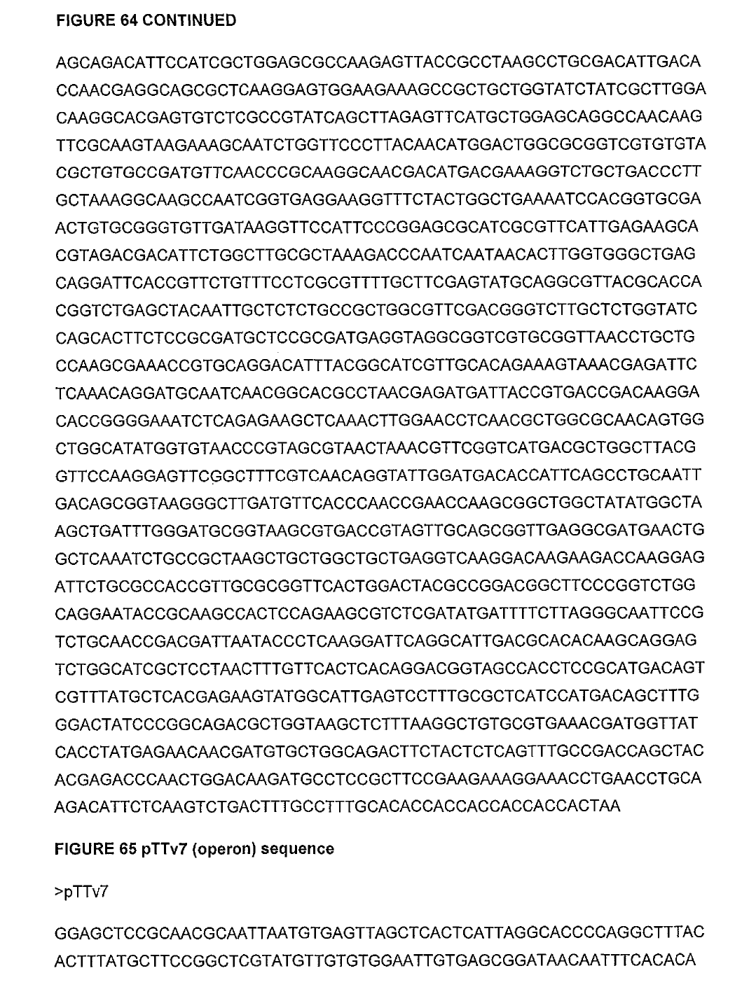

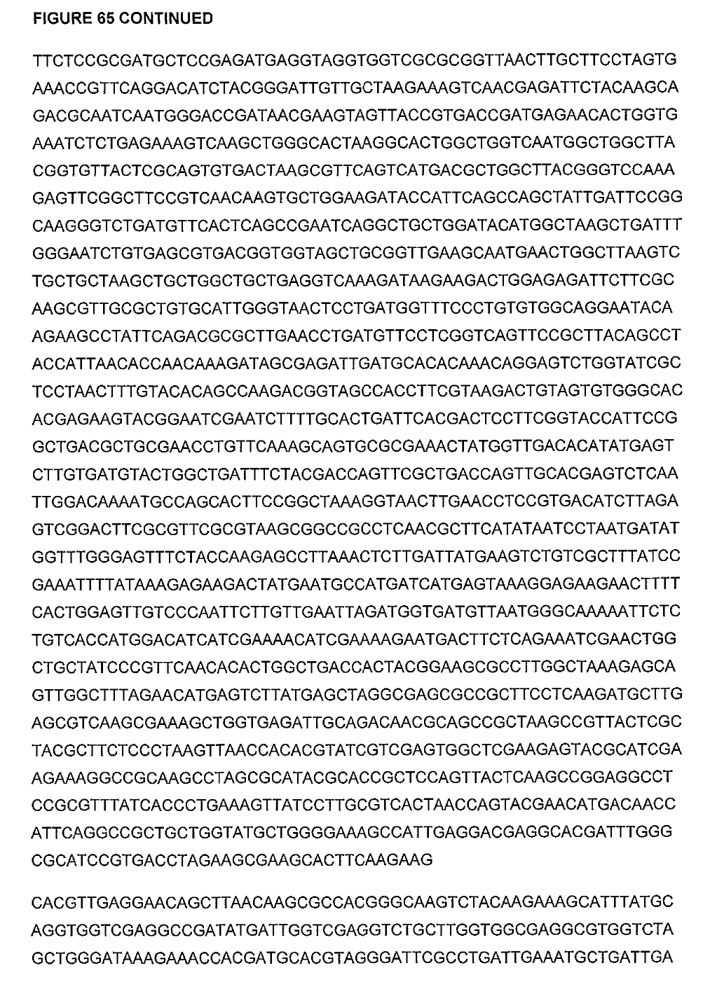

Improved 2^{nd} generation ligands have been discovered both in terms of in vivo and in vitro binding affinity. Specifically the pyrimido[4,5-d]pyrimidine-2,4-diamine A43 ligand demonstrates greatly improved activity against the M6″ when compared to the 1^{st} generation ligands (Amm) this is demonstrated clearly in the eGFP expression assay (FIG. 58), where dose-dependent control over eGFP expression is observed, further the maximum expression exceeds that of the WT (adddA) with adenine. The A43 ligand is again entirely selective for the M6″ riboswitch and demonstrates no affinity toward to the AddA(wt) riboswitch, this is demonstrated by the Isothermal titration calorimetry measurements (FIG. 59), where the A43 affinity for the aptamer of the M6″ riboswitch=189 nM. An additional 2^{nd} generation ligand discovered the 2,4-Diaminopteridine A44 interestingly shows approximately equal in vitro biding affinity to both the addA(wt) and M6″ riboswitch aptamers both ˜2 uM (FIG. 60). This indicates that by placement of the heterocyclic nitrogens in either positions 1-3 (A43), and 1-4 (A44), the selectivity of the ligand can be changed from M6″ selective to equally active against both addA and M6″ aptamers.

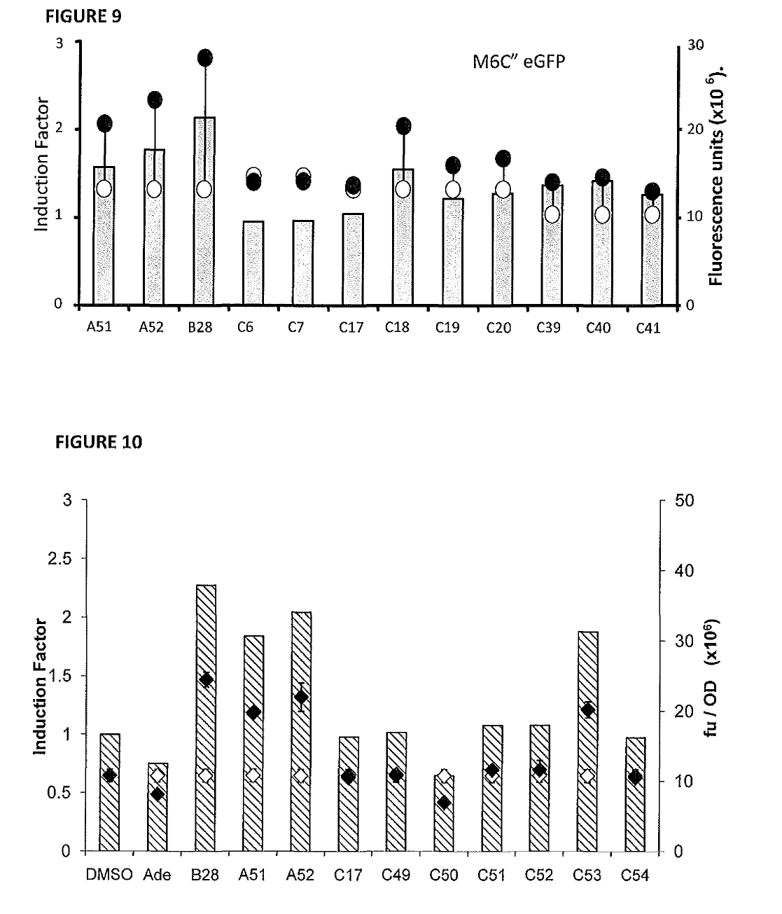

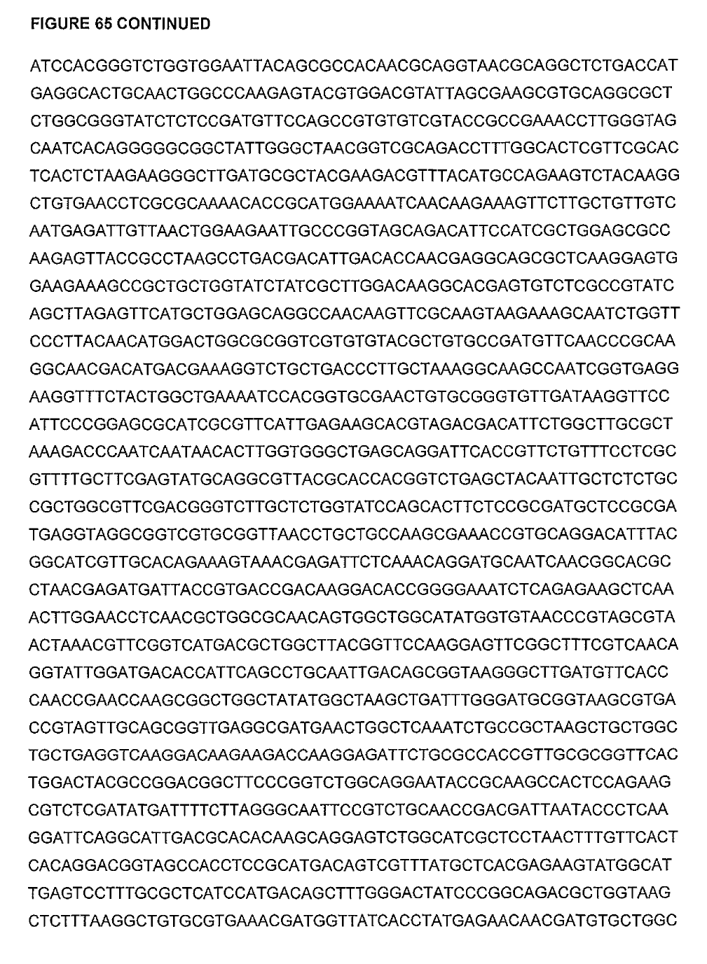

The inventors further found from in vivo and in vitro analysis that a number of guanosine nucleotide analogues induced gene expression when assayed against E. coli cells bearing a plasmid containing the riboswitch-reporter gene construct M6C″-eGFP (FIG. 9, 10). Isothermal titration calorimetry plots between the riboswitch aptamers M6C″ and ligands indicate K_{d} values range from 165-460 uM for ligands A52, C44, C39 shown (FIG. 61)

1-54. (canceled)

55. A system comprising
a) a first genetic construct comprising a first nucleic acid molecule encoding a riboswitch operably linked to a second nucleic acid molecule comprising a regulatory sequence; and optionally
b) a second genetic construct comprising a nucleic acid molecule comprising a coding sequence whose expression is capable of being regulated by a gene product of the second nucleic acid molecule.

56. A system according to claim 55, wherein the regulatory sequence of the second nucleic acid molecule encodes a polymerase, a transcription factor, an oscillator, a specificity factor, a repressor, an activator, or a regulatory RNA molecule, which serves to regulate expression of the coding sequence of the second genetic construct.

57. A system according to claim 55, wherein the regulatory sequence may encode an antisense sequence, which is targeted against a component which effects expression of the coding sequence of the second construct, for example a polymerase, a transcription factor, an oscillator, a specificity factor, a repressor, an activator, or a regulatory RNA molecule.

58. A system according to any one of claim 55, wherein the first genetic construct comprises a first RNA molecule comprising a riboswitch operably linked to a second RNA molecule which is, or encodes, a regulatory sequence.

59. A system according to claim 55, wherein the second genetic construct is DNA or RNA.

60. A system according to claim 55, wherein the first genetic construct comprises a spacer operably linked to the riboswitch.

61. A system according to claim 60, wherein the second genetic construct may independently comprise a nucleic acid molecule comprising a spacer sequence.


SEQUENCE LISTING (66 sequences)

SEQ ID NO: 1 - DNA, 18 bp, Unknown
  Eukaryotic (Kozak) consensus sequence
cccgccgccaccatggag

SEQ ID NO: 2 - DNA, 19 bp, Bacteriophage T7
taatacgactcactatagg

SEQ ID NO: 3 - DNA, 19 bp, Bacteriophage T3
aattaaccctcactaaagg

SEQ ID NO: 4 - RNA, 80 nt, Bacillus subtilis
acacgaccucauauaaucuugggaauauggcccauaaguuucuacccggcaaccguaaauugccggacuaugcaggaaag

SEQ ID NO: 5 - RNA, 78 nt, Bacillus subtilis
aggaacacucauauaaucgcguggauauggcacgcaaguuucuaccgggcaccguaaauguccgacuaugggugagca

SEQ ID NO: 6 - RNA, 78 nt, Bacillus subtilis
auuaucacuuguauaaccucaauaauaugguuugagggugucuaccaggaaccguaaaauccugauuacaaaauuuug

SEQ ID NO: 7 - RNA, 78 nt, Clostridium perfringens
auuuugcuucguauaacucuaaugauauggauuagaggucucuaccaagaaccgagaauucuugauuacgaagaaagc

SEQ ID NO: 8 - RNA, 78 nt, Vibrio vulnificus
ucaacgcuucauauaauccuaaugauaugguuugggaguuucuaccaagagccuuaaacucuugauuaugaagucugu

SEQ ID NO: 9 - DNA, 121 bp, Artificial
  Riboswitch AddA
ctggatcctcaacgcttcatataatcctaatgatatggtttgggagtttctaccaagagccttaaactcttgattatgaagtctgtcgctttatccgaaattttataaagagaagactatg

SEQ ID NO: 10 - DNA, 121 bp, Artificial Sequence
  Riboswitch M6
ctggatcctcaacgcttcatataatcctaatgatatggtttgggagcttccaccaagagccttaaactcttgattatgaagtctgtcgctttatccgaaattttataaagagaagactatg

SEQ ID NO: 11 - DNA, 121 bp, Artificial Sequence
  Riboswitch M6′
ctggatcctcaacgcttcatataatcctaatgatatggtttaggagcttccaccaagagccttaaactcttgattatgaagtctgtcgctttatccgaaattttataaagagaagactatg

SEQ ID NO: 12 - DNA, 121 bp, Artificial Sequence
  Riboswitch M6“
ctggatcctcaacgcttcatataatccgaatgatatggtttcggagcttccaccaagagccttaaactcttgattatgaagtctgtcgctttatccgaaattttataaagagaagactatg

SEQ ID NO: 13 - DNA, 121 bp, Artificial Sequence
  Riboswitch M6C
ctggatcctcaacgcttcatataatcctaatgatatggtttgggagcttccaccaagagccttaaactcttgactatgaagtctgtcgctttatccgaaattttataaagagaagactatg

SEQ ID NO: 14 - DNA, 121 bp, Artificial Sequence
  Riboswitch M6C′
ctggatcctcaacgcttcatataatcctaatgatatggtttaggagcttccaccaagagccttaaactcttgactatgaagtctgtcgctttatccgaaattttataaagagaagactatg

SEQ ID NO: 15 - DNA, 121 bp, Artificial Sequence
  Riboswitch M6C”
ctggatcctcaacgcttcatataatccgaatgatatggtttcggagcttccaccaagagccttaaactcttgactatgaagtctgtcgctttatccgaaattttataaagagaagactatg

SEQ ID NO: 16 - DNA, 330 bp, Bacillus subtilis
ataatgaaacgaaccgtcactatagagcgataattgatctaggaaccggggactgacttttttattgtcaagaaaaacatcattatggtaagatagcagaagtgaaaaaattgaagaaaatccgtgcgatatgcgggagaggttctagctacaccctctataaaaaactaaggacgagctgtatccttggatacggccttttttatgtttttctagagcaccttccgaaaaaaggtgtttttttgcgtgaattagctgtagcgatgtctctcgccggcgtttttattgcggagaaggagaggaatcatgaaaaaagaaaaagcaattgtc

SEQ ID NO: 17 - DNA, 34 bp, Artificial Sequence
  Consensus mutant sequence of a PreQ riboswitch
agagrttctagctacayyctctataaaarrctaa

SEQ ID NO: 18 - DNA, 34 bp, Unknown
  WT bacterial sequence
agaggttctagctacaccctctataaaaaactaa

SEQ ID NO: 19 - DNA, 34 bp, Artificial Sequence
  Mutant C17U
agaggttctagctacatcctctataaaaaactaa

SEQ ID NO: 20 - DNA, 34 bp, Artificial Sequence
  Mutant C18U
agaggttctagctacactctctataaaaaactaa

SEQ ID NO: 21 - DNA, 34 bp, Artificial Sequence
  Mutant G5A
agagattctagctacaccctctataaaaaactaa

SEQ ID NO: 22 - DNA, 34 bp, Artificial Sequence
  Mutant U6C
agaggctctagctacaccctctataaaaaactaa

SEQ ID NO: 23 - DNA, 34 bp, Artificial Sequence
  Mutant C17U, C18U
agaggttctagctacattctctataaaaaactaa

SEQ ID NO: 24 - DNA, 34 bp, Artificial Sequence
  Mutant G5A, C18U
agagattctagctacactctctataaaaaactaa

SEQ ID NO: 25 - DNA, 34 bp, Artificial Sequence
  Mutant U6C, C17U
agaggctctagctacatcctctataaaaaactaa

SEQ ID NO: 26 - DNA, 34 bp, Artificial Sequence
  Mutant G5A, C17U, C18U
agagattctagctacattctctataaaaaactaa

SEQ ID NO: 27 - DNA, 34 bp, Artificial Sequence
  Mutant U6C, C17U, A29G
agaggctctagctacatcctctataaaagactaa

SEQ ID NO: 28 - DNA, 34 bp, Artificial Sequence
  Mutant G5A, U6C, C17U, C18U
agagactctagctacattctctataaaaaactaa

SEQ ID NO: 29 - DNA, 486 bp, Artificial Sequence
  DNA encoding antisense RNA sequence complementing the drug target sequence RodZ
aggaggaattaaccatgcagtggtggtggtggtggtgccatggaacttccccggaataatgtcatagtttccttcctccaaggcggttaaataccttttttgaatctttgttgccgcttggagatcatccaatgacattgctttttcctctctggcttctttgagccggattcctagttcagtcacaacaaacctcgagcaccaccaccaccaccactgcatggttaattcctcctgaataacaagtattgttggcagcagctccagcgccttagatgctggctttggcatttccttttgaagattcatgccggagatcttttcagacacatcatcatgatacgtattagggatatcttttttatgctcctcaaacagctgatccgcatcaagcccaacggcttctgcatattgcttgatgaatgcccgaacataaaagtaaagagttttgcttaatcgcaaagctctttttattattatctttta

SEQ ID NO: 30 - DNA, 1397 bp, Artificial Sequence
  example of antibiotic drug target sequence controlled by PreQ riboswitch (PreQ1-MreB)
ataatgaaacgaaccgtcactatagagcgataattgatctaggaaccggggactgacttttttattgtcaagaaaaacatcattatggtaagatagcagaagtgaaaaaattgaagaaaatccgtgcgatatgcgggagaggttctagctacaccctctataaaaaactaaggacgagctgtatccttggatacggccttttttatgtttttctagagcaccttccgaaaaaaggtgtttttttgcgtgaattagctgtagcgatgtctctcgccggcgtttttattgcggagaaggagaggaatcatgaaaaaagaaaaagcaattgtcccagcttgttgatacactaatgcttttatatagggaaaaggtggtgaactactatgtttggaattggtgctagagaccttggtatagatcttggaactgcgaatacgcttgtttttgtaaaaggaaaaggaattgttgtgagagagccgtcagttgtcgctttgcagacggatacgaaatcgattgtcgctgtcggaaatgatgcgaaaaatatgattggacggacaccgggcaacgtggtggctcttcgcccgatgaaagacggcgttatcgctgattatgaaacaacggcgacgatgatgaaatattacatcaatcaggccataaaaaataaaggcatgtttgccagaaaaccatatgtaatggtatgtgtcccatcaggcattacagctgttgaagaacgcgctgttatcgatgcgacaagacaggcgggagcgcgtgacgcgtatccgattgaagagccttttgccgcagcaatcggagccaatctgccagtttgggaaccgactggaagcatggttgttgatatcgggggcggtacgacagaagttgcgattatttccctcggaggcatcgtaacgtctcagtcaatccgtgtagccggtgatgagatggatgacgcgattatcaactacatcagaaaaacgtacaatctgatgatcggtgaccgtacggctgaagcgattaaaatggaaatcggatctgcagaagctcctgaagaatccgacaacatggaaatccgcggccgcgatttgctcacaggtttgccgaaaacaattgaaattacaggaaaagagatttctaacgctctacgcgacactgtatctacaattgtcgaagcagtgaagagcacactcgaaaaaacaccgcctgagcttgcagcagatatcatggacagaggtatagtgttaaccggcggcggagcgcttttgcgcaatttggacaaagtcatcagcgaagaaacaaaaatgccggtccttatcgccgaagatccgcttgattgtgtagcgatcggaacagggaaagcactggagcacatccatcttttcaaagggaaaactagataa

SEQ ID NO: 31 - DNA, 105 bp, Artificial
  Spacer sequence GFP 105nt
atgaatggcatgctcatgagtaaaggagaagaacttttcactggagttgtcccaattcttgttgaattagatggtgatgttaatgggtacaaattttctgtcagt

SEQ ID NO: 32 - DNA, 90 bp, Artificial
  Spacer sequence gfp_90
atgaatgccatgatcatgagtaaaggagaagaacttttcactggagttgtcccaattcttgttgaattagatggtgatgttaatgggccc

SEQ ID NO: 33 - DNA, 75 bp, Artificial
  Spacer sequence gfp_75
atgaatgccatgatcatgagtaaaggagaagaacttttcactggagttgtcccaattcttgttgaattagatggt

SEQ ID NO: 34 - DNA, 60 bp, Artificial
  Spacer sequence gfp_60
atgaatgccatgatcatgagtaaaggagaagaacttttcactggagttgtcccaattctt

SEQ ID NO: 35 - DNA, 45 bp, Artificial
  Spacer sequence gfp_45
atgaatgccatgatcatgagtaaaggagaagaacttttcactgga

SEQ ID NO: 36 - DNA, 30 bp, Artificial
  Spacer sequence gfp_30
atgaatgccatgatcatgagtaaaggagaa

SEQ ID NO: 37 - DNA, 105 bp, Unknown
  Spacer sequence GFP_codon optimizedvariant
atgattgaacaagatggattgcacgcaggttctccggccgcttgggtggagaggctattcggctatgactgggcacaacagacaatcggctgctctgatgccgcc

SEQ ID NO: 38 - DNA, 105 bp, Unknown
  Spacer sequence DSred
atggcctcctccgaggacgtcatcaaggagttcatgcgcttcaaggtgcgcatggagggctccgtgaacggccacgagttcgagatcgagggcgagggcgagggc

SEQ ID NO: 39 - DNA, 105 bp, Unknown
  Spacer sequence lacZ
atggccatgattacggattcactggccgtcgttttacaacgtcgtgactgggaaaaccctggcgttacccaacttaatcgccttgcagcacatccccctttcgcc

SEQ ID NO: 40 - DNA, 105 bp, Unknown
  Spacer sequence T7
atgaacacgattaacatcgctaagaacgacttctctgacatcgaactggctgctatcccgttcaacactctggctgaccattacggtgagcgtttagctcgcgaa

SEQ ID NO: 41 - DNA, 105 bp, Unknown; identical to SEQ ID NO: 40
  Spacer sequence T3

SEQ ID NO: 42 - DNA, 105 bp, Unknown
  Spacer sequence SP6
atgcaagatttacacgctatccagcttcaattagaagaagagatgtttaatggtggcattcgtcgcttcgaagcagatcaacaacgccagattgcagcaggtagc

SEQ ID NO: 43 - DNA, 105 bp, Artificial Sequence
  Example spacer used to separate tandem riboswitches
atgaatgccatgatcatgagtaaaggagaagaacttttcactggagttgtcccaattcttgttgaattagatggtgatgttaatgggtacaaattttctgtcagg

SEQ ID NO: 44 - DNA, 210 bp, Artificial Sequence
  Example spacer used to separate tandem riboswitches
atgaatggcatgctcatgagtaaaggagaagaacttttcactggagttgtcccaattcttgttgaattagatggtgatgttaatgggtacaaattttctgtcagtctccaagctggacatcacctcccacaacgaggactacaccatcgtggagcagtacgagcgcgccgagggccgccaccacctgttcctgtaggcggccgcctcaac

SEQ ID NO: 45 - DNA, 234 bp, Artificial Sequence
  Example spacer used to separate tandem riboswitches
atgaatggcatgctcatgagtaaaggagaagaacttttcactggagttgtcccaattcttgttgaattagatggtgatgttaatgggtacaaattttctgtcagtataaccccgctcttacacattccactccaagctggacatcacctcccacaacgaggactacaccatcgtggagcagtacgagcgcgccgagggccgccaccacctgttcctgtaggcggccgcctcaac

SEQ ID NO: 46 - DNA, 1788 bp, Artificial Sequence
  Operon sequence, composed of lacUV5 promoter/operator, M6 riboswitch, DsRED gene, AddA riboswitch, eGFP gene
aattgtgagcggataacaatttcacacaggaaacagctggatcctcaacgcttcatataatccgaatgatatggtttcggagcttccaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaggcggccgcctcaacgcttcatataatcctaatgatatggtttgggagtttctaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaggggagagggtgaaggtgatgcaacatacggaaaacttacccttaaatttatttgcactactgggaagctacctgttccatggccaacacttgtcactactttgacttatggtgtacaatgcttctcaagatacccagatcacatgaaacagcatgactttctcaagagtgccatgcccgaaggttatgtacaggaaagaactatattttacaaagatgacgggaactacaagacacgtgctgaagtcaagtttgagggtgatacccttgttaatagaatcgagttaaaaggtattgattttaaagaagatggaaacattcttggacacaaaatggaatacaactataactcacataatgtatacatcatgggagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacacaatctgccctttcccaagatccccacggaaagagagatcacatggtccttcttgagtttgttacatctgctgggattacacatggcatggatgaactatacaaatag

SEQ ID NO: 47 - DNA, 1943 bp, Artificial Sequence
  Dual Promoter sequence, composed of lacUV5 promoter/operator, M6 riboswitch, DsRED gene, lambda tR1 terminator, lacUV5 promoter/operator, AddA riboswitch, eGFP gene promoter
aattgtgagcggataacaatttcacacaggaaacagctggatcctcaacgcttcatataatccgaatgatatggtttcggagcttccaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtaggcggccgccataaccccgctcttacacattccagccccgcaacgcaattaatgtgagttagctcactcattaggcaccccaggctttacactttatgcttccggctcgtatgttgtgtggaattgtgagcggataacaatttcacacaggaaacagctggatcctcaacgcttcatataatcctaatgatatggtttgggagtttctaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaggggagagggtgaaggtgatgcaacatacggaaaacttacccttaaatttatttgcactactgggaagctacctgttccatggccaacacttgtcactactttgacttatggtgtacaatgcttctcaagatacccagatcacatgaaacagcatgactttctcaagagtgccatgcccgaaggttatgtacaggaaagaactatattttacaaagatgacgggaactacaagacacgtgctgaagtcaagtttgagggtgatacccttgttaatagaatcgagttaaaaggtattgattttaaagaagatggaaacattcttggacacaaaatggaatacaactataactcacataatgtatacatcatgggagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacacaatctgccctttcccaagatccccacggaaagagagatcacatggtccttcttgagtttgttacatctgctgggattacacatggcatggatgaactatacaaatag

SEQ ID NO: 48 - DNA, 2858 bp, Artificial Sequence
  Orthogonal Riboswitch T7 sequence, composed of lacUV5 promoter/operator, M6“ riboswitch, T7 RNA polymerase
cattaggcaccccaggctttacactttatgcttccggctcgtatgttgtgtggaattgtgagcggataacaatttcacacaggaaacagctggatcctcaacgcttcatataatccgaatgatatggtttcggagcttccaccaagagccttaaactcttgattatgaagtctgtcgctttatccgaaattttataaagagaagactatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 49 - DNA, 2912 bp, Artificial Sequence
  Orthogonal Riboswitch spacer T7 sequence, composed of lacUV5 promoter/operator, M6” riboswitch, spacer sequence, T7 RNA polymerase
aattgtgagcggataacaatttcacacaggaaacagctggatcctcaacgcttcatataatccgaatgatatggtttcggagcttccaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggccacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaag

SEQ ID NO: 50 - DNA, 165 bp, Artificial Sequence
  Spacer sequence sp165
atgaatgccatgatcatgagtaaaggagaagaacttttcactggagttgtcccaattcttgttgaattagatggtgatgttaatgggtacaaattttctgtcagtggagagggtgaaggtgatgcaacatacggaaaacttacccttaaatttatttgcactacc

SEQ ID NO: 51 - DNA, 150 bp, Artificial Sequence
  Spacer sequence sp150
atgaatgccatgatcatgagtaaaggagaagaacttttcactggagttgtcccaattcttgttgaattagatggtgatgttaatgggtacaaattttctgtcagtggagagggtgaaggtgatgcaacatacggaaaacttacccttaac

SEQ ID NO: 52 - DNA, 135 bp, Artificial Sequence
  Spacer sequence sp135
atgaatgccatgatcatgagtaaaggagaagaacttttcactggagttgtcccaattcttgttgaattagatggtgatgttaatgggtacaaattttctgtcagtggagagggtgaaggtgatgcaacatacggc

SEQ ID NO: 53 - DNA, 120 bp, Artificial Sequence
  Spacer sequence sp120
atgaatgccatgatcatgagtaaaggagaagaacttttcactggagttgtcccaattcttgttgaattagatggtgatgttaatgggtacaaattttctgtcagtggagagggtgaaggc

SEQ ID NO: 54 - DNA, 105 bp, Artificial Sequence
  Spacer sequence sp105
atgaatgccatgatcatgagtaaaggagaagaacttttcactggagttgtcccaattcttgttgaattagatggtgatgttaatgggcaaaaattttctgtcacc

SEQ ID NO: 55 - DNA, 102 bp, Artificial Sequence
  Spacer sequence sp102
atgaatgccatgatcatgagtaaaggagaagaacttttcactggagttgtcccaattcttgttgaattagatggtgatgttaatgggcaaaaattctctgcc

SEQ ID NO: 56 - DNA, 99 bp, Artificial Sequence
  Spacer sequence sp99
atgaatgccatgatcatgagtaaaggagaagaacttttcactggagttgtcccaattcttgttgaattagatggtgatgttaatgggcaaaaattctcc

SEQ ID NO: 57 - DNA, 96 bp, Artificial Sequence
  Spacer sequence sp96
atgaatgccatgatcatgagtaaaggagaagaacttttcactggagttgtcccaattcttgttgaattagatggtgatgttaatgggcaaaaatcc

SEQ ID NO: 58 - DNA, 93 bp, Artificial Sequence
  Spacer sequence sp93
atgaatgccatgatcatgagtaaaggagaagaacttttcactggagttgtcccaattcttgttgaattagatggtgatgttaatgggcaaacc

SEQ ID NO: 59 - DNA, 90 bp, Artificial Sequence; identical to SEQ ID NO: 32
  Spacer sequence sp90

SEQ ID NO: 60 - DNA, 75 bp, Artificial Sequence
  Spacer sequence sp75
atgaatgccatgatcatgagtaaaggagaagaacttttcactggagttgtcccaattcttgttgaattagatgcc

SEQ ID NO: 61 - DNA, 60 bp, Artificial Sequence
  Spacer sequence sp60
atgaatgccatgatcatgagtaaaggagaagaacttttcactggagttgtcccaattccc

SEQ ID NO: 62 - DNA, 45 bp, Artificial Sequence
  Spacer sequence sp45
atgaatgccatgatcatgagtaaaggagaagaacttttcactgcc

SEQ ID NO: 63 - DNA, 30 bp, Artificial Sequence
  Spacer sequence sp30
atgaatgccatgatcatgagtaaaggagcc

SEQ ID NO: 64 - DNA, 3024 bp, Artificial Sequence
  pMAST-h6 sequence, composed of lacUV5 promoter/operator, AddA riboswitch, spacer, T3 RNA polymerase with a C-his6 tag
aattctctagacgcaacgcaattaatgtgagttagctcactcattaggcaccccaggctttacactttatgcttccggctcgtatgttgtgtggaattgtgagcggataacaatttcacacaggaaacagctggatcctcaacgcttcatataatcctaatgatatggtttgggagtttctaccaagagcctaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggacatcatcgaaaacatcgaaaagaatgacttctcagaaatcgaactggctgctatcccgttcaacacactggctgaccactacggaagcgccttggctaaagagcagttggctttagaacatgagtcttatgagctaggcgagcgccgcttcctcaagatgcttgagcgtcaagcgaaagctggtgagattgcagacaacgcagccgctaagccgttactcgctacgcttctccctaagttaaccacacgtatcgtcgagtggctcgaagagtacgcatcgaagaaaggccgcaagcctagcgcatacgcaccgctccagttactcaagccggaggcctccgcgtttatcaccctgaaagttatccttgcgtcactaaccagtacgaacatgacaaccattcaggccgctgctggtatgctggggaaagccattgaggacgaggcacgatttgggcgcatccgtgacctagaagcgaagcacttcaagaagcacgttgaggaacagcttaacaagcgccacgggcaagtctacaagaaagcatttatgcaggtggtcgaggccgatatgattggtcgaggtctgcttggtggcgaggcgtggtctagctgggataaagaaaccacgatgcacgtagggattcgcctgattgaaatgctgattgaatccacgggtctggtggaattacagcgccacaacgcaggtaacgcaggctctgaccatgaggcactgcaactggcccaagagtacgtggacgtattagcgaagcgtgcaggcgctctggcgggtatctctccgatgttccagccgtgtgtcgtaccgccgaaaccttgggtagcaatcacagggggcggctattgggctaacggtcgcagacctttggcactcgttcgcactcactctaagaagggcttgatgcgctacgaagacgtttacatgccagaagtctacaaggctgtgaacctcgcgcaaaacaccgcatggaaaatcaacaagaaagttcttgctgttgtcaatgagattgttaactggaagaattgcccggtagcagacattccatcgctggagcgccaagagttaccgcctaagcctgcgacattgacaccaacgaggcagcgctcaaggagtggaagaaagccgctgctggtatctatcgcttggacaaggcacgagtgtctcgccgtatcagcttagagttcatgctggagcaggccaacaagttcgcaagtaagaaagcaatctggttcccttacaacatggactggcgcggtcgtgtgtacgctgtgccgatgttcaacccgcaaggcaacgacatgacgaaaggtctgctgacccttgctaaaggcaagccaatcggtgaggaaggtttctactggctgaaaatccacggtgcgaactgtgcgggtgttgataaggttccattcccggagcgcatcgcgttcattgagaagcacgtagacgacattctggcttgcgctaaagacccaatcaataacacttggtgggctgagcaggattcaccgttctgtttcctcgcgttttgcttcgagtatgcaggcgttacgcaccacggtctgagctacaattgctctctgccgctggcgttcgacgggtcttgctctggtatccagcacttctccgcgatgctccgcgatgaggtaggcggtcgtgcggttaacctgctgccaagcgaaaccgtgcaggacatttacggcatcgttgcacagaaagtaaacgagattctcaaacaggatgcaatcaacggcacgcctaacgagatgattaccgtgaccgacaaggacaccggggaaatctcagagaagctcaaacttggaacctcaacgctggcgcaacagtggctggcatatggtgtaacccgtagcgtaactaaacgttcggtcatgacgctggcttacggttccaaggagttcggctttcgtcaacaggtattggatgacaccattcagcctgcaattgacagcggtaagggcttgatgttcacccaaccgaaccaagcggctggctatatggctaagctgatttgggatgcggtaagcgtgaccgtagttgcagcggttgaggcgatgaactggctcaaatctgccgctaagctgctggctgctgaggtcaaggacaagaagaccaaggagattctgcgccaccgttgcgcggttcactggactacgccggacggcttcccggtctggcaggaataccgcaagccactccagaagcgtctcgatatgattttcttagggcaattccgtctgcaaccgacgattaataccctcaaggattcaggcattgacgcacacaagcaggagtctggcatcgctcctaactttgttcactcacaggacggtagccacctccgcatgacagtcgtttatgctcacgagaagtatggcattgagtcctttgcgctcatccatgacagctttgggactatcccggcagacgctggtaagctctttaaggctgtgcgtgaaacgatggttatcacctatgagaacaacgatgtgctggcagacttctactctcagtttgccgaccagctacacgagacccaactggacaagatgcctccgcttccgaagaaaggaaacctgaacctgcaagacattctcaagtctgactttgcctttgcacaccaccaccaccaccactaa

SEQ ID NO: 65 - DNA, 5898 bp, Artificial Sequence
  pTTv7 (operon) sequence, composed of lacUV5 promoter/operator, M6“ riboswitch, spacer, T7 RNAP gene, AddA riboswitch, spacer T3 RNAP gene
ggagctccgcaacgcaattaatgtgagttagctcactcattaggcaccccaggctttacactttatgcttccggctcgtatgttgtgtggaattgtgagcggataacaatttcacacaggaaacagctggatcctcaacgcttcatataatccgaatgatatggtttcggagcttccaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggccacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcggccgcctcaacgcttcatataatcctaatgatatggtttgggagtttctaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggacatcatcgaaaacatcgaaaagaatgacttctcagaaatcgaactggctgctatcccgttcaacacactggctgaccactacggaagcgccttggctaaagagcagttggctttagaacatgagtcttatgagctaggcgagcgccgcttcctcaagatgcttgagcgtcaagcgaaagctggtgagattgcagacaacgcagccgctaagccgttactcgctacgcttctccctaagttaaccacacgtatcgtcgagtggctcgaagagtacgcatcgaagaaaggccgcaagcctagcgcatacgcaccgctccagttactcaagccggaggcctccgcgtttatcaccctgaaagttatccttgcgtcactaaccagtacgaacatgacaaccattcaggccgctgctggtatgctggggaaagccattgaggacgaggcacgatttgggcgcatccgtgacctagaagcgaagcacttcaagaagcacgttgaggaacagcttaacaagcgccacgggcaagtctacaagaaagcatttatgcaggtggtcgaggccgatatgattggtcgaggtctgcttggtggcgaggcgtggtctagctgggataaagaaaccacgatgcacgtagggattcgcctgattgaaatgctgattgaatccacgggtctggtggaattacagcgccacaacgcaggtaacgcaggctctgaccatgaggcactgcaactggcccaagagtacgtggacgtattagcgaagcgtgcaggcgctctggcgggtatctctccgatgttccagccgtgtgtcgtaccgccgaaaccttgggtagcaatcacagggggcggctattgggctaacggtcgcagacctttggcactcgttcgcactcactctaagaagggcttgatgcgctacgaagacgtttacatgccagaagtctacaaggctgtgaacctcgcgcaaaacaccgcatggaaaatcaacaagaaagttcttgctgttgtcaatgagattgttaactggaagaattgcccggtagcagacattccatcgctggagcgccaagagttaccgcctaagcctgacgacattgacaccaacgaggcagcgctcaaggagtggaagaaagccgctgctggtatctatcgcttggacaaggcacgagtgtctcgccgtatcagcttagagttcatgctggagcaggccaacaagttcgcaagtaagaaagcaatctggttcccttacaacatggactggcgcggtcgtgtgtacgctgtgccgatgttcaacccgcaaggcaacgacatgacgaaaggtctgctgacccttgctaaaggcaagccaatcggtgaggaaggtttctactggctgaaaatccacggtgcgaactgtgcgggtgttgataaggttccattcccggagcgcatcgcgttcattgagaagcacgtagacgacattctggcttgcgctaaagacccaatcaataacacttggtgggctgagcaggattcaccgttctgtttcctcgcgttttgcttcgagtatgcaggcgttacgcaccacggtctgagctacaattgctctctgccgctggcgttcgacgggtcttgctctggtatccagcacttctccgcgatgctccgcgatgaggtaggcggtcgtgcggttaacctgctgccaagcgaaaccgtgcaggacatttacggcatcgttgcacagaaagtaaacgagattctcaaacaggatgcaatcaacggcacgcctaacgagatgattaccgtgaccgacaaggacaccggggaaatctcagagaagctcaaacttggaacctcaacgctggcgcaacagtggctggcatatggtgtaacccgtagcgtaactaaacgttcggtcatgacgctggcttacggttccaaggagttcggctttcgtcaacaggtattggatgacaccattcagcctgcaattgacagcggtaagggcttgatgttcacccaaccgaaccaagcggctggctatatggctaagctgatttgggatgcggtaagcgtgaccgtagttgcagcggttgaggcgatgaactggctcaaatctgccgctaagctgctggctgctgaggtcaaggacaagaagaccaaggagattctgcgccaccgttgcgcggttcactggactacgccggacggcttcccggtctggcaggaataccgcaagccactccagaagcgtctcgatatgattttcttagggcaattccgtctgcaaccgacgattaataccctcaaggattcaggcattgacgcacacaagcaggagtctggcatcgctcctaactttgttcactcacaggacggtagccacctccgcatgacagtcgtttatgctcacgagaagtatggcattgagtcctttgcgctcatccatgacagctttgggactatcccggcagacgctggtaagctctttaaggctgtgcgtgaaacgatggttatcacctatgagaacaacgatgtgctggcagacttctactctcagtttgccgaccagctacacgagacccaactggacaagatgcctccgcttccgaagaaaggaaacctgaacctgcaagacattctcaagtctgactttgcctttgcacaccaccaccaccaccactaa

SEQ ID NO: 66 - DNA, 6053 bp, Artificial Sequence
  pTTv9 (dual promoter) sequence, composed of lacUV5 promoter/operator, M6” riboswitch, spacer, T7 RNAP gene, lambda tR1 terminator, lacUV5 promoter/operator, AddA riboswitch, spacer T3 RNAP gene
ggagctccgcaacgcaattaatgtgagttagctcactcattaggcaccccaggctttacactttatgcttccggctcgtatgttgtgtggaattgtgagcggataacaatttcacacaggaaacagctggatcctcaacgcttcatataatccgaatgatatggtttcggagcttccaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggccacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcggccgccataaccccgctcttacacattccagccccgcaacgcaattaatgtgagttagctcactcattaggcaccccaggctttacactttatgcttccggctcgtatgttgtgtggaattgtgagcggataacaatttcacacaggaaacagctggatcctcaacgcttcatataatcctaatgatatggtttgggagtttctaccaagagccttaaactcttgattatgaagtctgtcgctttatccgaaattttataaagagaagactatgaatgccatgatcatgagtaaaggagaagaacttttcactggagttgtcccaattcttgttgaattagatggtgatgttaatgggcaaaaattctctgtcaccatggacatcatcgaaaacatcgaaaagaatgacttctcagaaatcgaactggctgctatcccgttcaacacactggctgaccactacggaagcgccttggctaaagagcagttggctttagaacatgagtcttatgagctaggcgagcgccgcttcctcaagatgcttgagcgtcaagcgaaagctggtgagattgcagacaacgcagccgctaagccgttactcgctacgcttctccctaagttaaccacacgtatcgtcgagtggctcgaagagtacgcatcgaagaaaggccgcaagcctagcgcatacgcaccgctccagttactcaagccggaggcctccgcgtttatcaccctgaaagttatccttgcgtcactaaccagtacgaacatgacaaccattcaggccgctgctggtatgctggggaaagccattgaggacgaggcacgatttgggcgcatccgtgacctagaagcgaagcacttcaagaagcacgttgaggaacagcttaacaagcgccacgggcaagtctacaagaaagcatttatgcaggtggtcgaggccgatatgattggtcgaggtctgcttggtggcgaggcgtggtctagctgggataaagaaaccacgatgcacgtagggattcgcctgattgaaatgctgattgaatccacgggtctggtggaattacagcgccacaacgcaggtaacgcaggctctgaccatgaggcactgcaactggcccaagagtacgtggacgtattagcgaagcgtgcaggcgctctggcgggtatctctccgatgttccagccgtgtgtcgtaccgccgaaaccttgggtagcaatcacagggggcggctattgggctaacggtcgcagacctttggcactcgttcgcactcactctaagaagggcttgatgcgctacgaagacgtttacatgccagaagtctacaaggctgtgaacctcgcgcaaaacaccgcatggaaaatcaacaagaaagttcttgctgttgtcaatgagattgttaactggaagaattgcccggtagcagacattccatcgctggagcgccaagagttaccgcctaagcctgacgacattgacaccaacgaggcagcgctcaaggagtggaagaaagccgctgctggtatctatcgcttggacaaggcacgagtgtctcgccgtatcagcttagagttcatgctggagcaggccaacaagttcgcaagtaagaaagcaatctggttcccttacaacatggactggcgcggtcgtgtgtacgctgtgccgatgttcaacccgcaaggcaacgacatgacgaaaggtctgctgacccttgctaaaggcaagccaatcggtgaggaaggtttctactggctgaaaatccacggtgcgaactgtgcgggtgttgataaggttccattcccggagcgcatcgcgttcattgagaagcacgtagacgacattctggcttgcgctaaagacccaatcaataacacttggtgggctgagcaggattcaccgttctgtttcctcgcgttttgcttcgagtatgcaggcgttacgcaccacggtctgagctacaattgctctctgccgctggcgttcgacgggtcttgctctggtatccagcacttctccgcgatgctccgcgatgaggtaggcggtcgtgcggttaacctgctgccaagcgaaaccgtgcaggacatttacggcatcgttgcacagaaagtaaacgagattctcaaacaggatgcaatcaacggcacgcctaacgagatgattaccgtgaccgacaaggacaccggggaaatctcagagaagctcaaacttggaacctcaacgctggcgcaacagtggctggcatatggtgtaacccgtagcgtaactaaacgttcggtcatgacgctggcttacggttccaaggagttcggctttcgtcaacaggtattggatgacaccattcagcctgcaattgacagcggtaagggcttgatgttcacccaaccgaaccaagcggctggctatatggctaagctgatttgggatgcggtaagcgtgaccgtagttgcagcggttgaggcgatgaactggctcaaatctgccgctaagctgctggctgctgaggtcaaggacaagaagaccaaggagattctgcgccaccgttgcgcggttcactggactacgccggacggcttcccggtctggcaggaataccgcaagccactccagaagcgtctcgatatgattttcttagggcaattccgtctgcaaccgacgattaataccctcaaggattcaggcattgacgcacacaagcaggagtctggcatcgctcctaactttgttcactcacaggacggtagccacctccgcatgacagtcgtttatgctcacgagaagtatggcattgagtcctttgcgctcatccatgacagctttgggactatcccggcagacgctggtaagctctttaaggctgtgcgtgaaacgatggttatcacctatgagaacaacgatgtgctggcagacttctactctcagtttgccgaccagctacacgagacccaactggacaagatgcctccgcttccgaagaaaggaaacctgaacctgcaagacattctcaagtctgactttgcctttgcacaccaccaccaccaccactaa